(19) United States
(12) Patent Application Publication
(10) Pub. No.: US 2026/0248968 A1
(43) Pub. Date: Aug. 27, 2026

(54) ELEMENTS FOR DE-TARGETING GENE EXPRESSION IN DORSAL ROOT GANGLION AND/OR LIVER

(71) Applicant: ENCODED THERAPEUTICS, INC., South San Francisco, CA (US)

(72) Inventors: Anne TANENHAUS, South San Francisco, CA (US); Ben ZHAO, South San Francisco, CA (US); Serena LIU, South San Francisco, CA (US); Puja DHANOTA, South San Francisco, CA (US); Raghavendra HOSUR, South San Francisco, CA (US); Steven TAN, South San Francisco, CA (US); John MCLAUGHLIN, South San Francisco, CA (US); Martin MOORHEAD, South San Francisco, CA (US); Sheila SEARS, South San Francisco, CA (US); Yosr BOUHLAL, South San Francisco, CA (US); Nathan MOERKE, South San Francisco, CA (US); Camille G. ARTUR, South San Francisco, CA (US); Mitchell LOPEZ, South San Francisco, CA (US); Tulasi Indrasinh SOLANKI, South San Francisco, CA (US); Greg LUCEY, South San Francisco, CA (US)

(21) Appl. No.: 19/107,531

(22) PCT Filed: Sep. 22, 2023
(86) PCT No.: PCT/US2023/074878

§ 371 Date: Feb. 27, 2025

Related U.S. Application Data
(60) Provisional application No. 63/410,161, filed on Sep. 26, 2022.
(60) Provisional application No. 63/412,119, filed on Sep. 30, 2022.
(60) Provisional application No. 63/414,873, filed on Oct. 10, 2022.
(60) Provisional application No. 63/464,772, filed on May 8, 2023.

(51) Int. Cl. A61K 48/00; A61K 9/00; C12N 15/86

(52) U.S. Cl. CPC A61K 48/0058; A61K 9/0085; A61K 48/0066; A61K 48/0075; C12N 15/86; C12N 2750/14143

(57) ABSTRACT

The present disclosure provides sequences that reduce expression of an operably linked transgene in dorsal root ganglion cells and/or liver cells. In some aspects, the sequences can be employed in gene therapy vectors to de-target expression of a therapeutic transgene in dorsal root ganglion and/or liver cells of a subject.

### CROSS-REFERENCING

This application claims the benefit of U.S. provisional application Ser. No. 63/410,161, filed on Sep. 26, 2022, 63/464,772, filed on May 8, 2022, 63/414,873 filed on Oct. 10, 2022, and 63/412,119, filed on Sep. 30, 2022, which applications are incorporated by reference herein.

### INCORPORATION BY REFERENCE OF SEQUENCE LISTING PROVIDED AS A SEQUENCE LISTING XML FILE

A Sequence Listing is provided herewith as a Sequence Listing XML, ENCO-006WO_SEQ_LIST, created on Sep. 22, 2023, and having a size of 141,924 bytes. The contents of the Sequence Listing XML are incorporated herein by reference in their entirety.

### INTRODUCTION

Gene therapy has enormous potential for the treatment of human diseases, particularly diseases that have an underlying genetic cause. In some gene therapy strategies, a therapeutic payload may be recombinantly expressed in a target cell that lacks or has a reduced amount or dysfunctional version of an essential protein. Expression of the therapeutic payload in the cells rescues those cells, thereby treating the disease. In one example, Tay-Sachs disease (which is recessively inherited and caused by mutations in the HEXA gene, which is on chromosome 15), can be successfully treated by expressing a functional version of hexA in the brain using adeno-associated virus (AAV) gene therapy.

One of the challenges in gene therapy is how to deliver a therapeutic payload to a specific tissue and not others. For example, some therapeutic payloads that have a positive effect in one tissue may have an adverse effect in another tissue. As such, administrating a gene therapy that targets diseased cells in one tissue may cause side-effects in another. In some cases, the clinical use of a gene therapy may even be limited by its off-site effects, rather than the on-site effects.

In view of the above, there is a general need for tools for increasing the tissue specificity of a gene therapy.

### SUMMARY

Provided herein, among other things, is a nucleic acid cassette comprising a transgene encoding an RNA transcript, e.g., an mRNA, wherein the RNA transcript comprises a sequence of (i) any of SEQ ID NOS. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii). These sequences reduce the expression of the transgene in cells of the dorsal root ganglion (DRG) relative to other cells or tissues (e.g., other cells in the brain) and thus may be employed in a variety of gene therapy strategies that target cells that are not in the DRG.

Also provided is a nucleic acid cassette comprising a therapeutic transgene encoding an RNA transcript, wherein the RNA transcript comprises a sequence of (i) any SEQ ID NOS. 65, 110, and 112, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), wherein the sequence decreases expression of the RNA transcript in liver cells.

Also provided is a nucleic acid cassette comprising a therapeutic transgene encoding an RNA transcript that comprises a first sequence that de-targets expression in dorsal root ganglion (DRG) cells and a second sequence that de-targets expression in liver cells. Addition of the first and second sequences result in decreased expression of the RNA transcript or a polypeptide encoded by the same in DRG and liver cells relative to a target tissue, e.g., GABAergic cells.

In certain aspects, incorporation of one or more of these sequences results in an improved safety profile of a gene therapy by reducing or eliminating toxicity to non-target cells (e.g., DRG and/or liver cells) caused by expression of the transgene in these cells.

In some embodiments, the nucleic acid cassette is an expression cassette, wherein the expression cassette may comprise, in operable linkage, a promoter, a coding sequence, one or more de-targeting sequences described above, and a terminator. In some embodiments, at least one sequence present in the expression cassette is heterologous to another one of the sequences in the expression cassette. For example, in some embodiments, an expression cassette of the present disclosure comprises a promoter that is heterologous to an operably linked coding sequence. In some embodiments, the expression cassette may further comprise an enhancer and/or an intron.

As would be apparent, in embodiments in which the RNA transcript encoded by a transgene is an mRNA that encodes a therapeutic protein and comprises a de-targeting sequence, the mRNA encoded by the transgene will contain both a coding sequence for the therapeutic protein and the de-targeting sequence, such that the mRNA that is expressed contains both the coding sequence and the DRG de-targeting sequence in the same transcript. While the mechanism may be unclear, it is understood that the de-targeting element may target RNA transcripts, e.g., mRNA molecules, that contain that element for preferential degradation in non-target cells.

It is noted that synthetic RNA molecules that include the de-targeting sequences disclosed herein can also be inactivated in the specified tissue (e.g., DRG, liver, or both) when introduced into such cells, e.g., the cells of a subject. For example, a synthetic antisense RNA that includes one or more DRG de-targeting elements of the disclosure, one or more liver de-targeting elements of the disclosure, or a combination of both, will have reduced activity in the de-targeted tissue(s) when administered to a subject. No limitation in this regard is intended.

In some embodiments, the promoter of the expression cassette may be selective for cells in a particular tissue (e.g., the target tissue) but it also drives transgene expression in the DRG and/or liver cells. In some embodiments, the promoter may be a CNS selective promoter, e.g., a promoter selected from the group consisting of: Ca²⁺/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters

In any embodiment, the sequence may be in a 3′ UTR, a 5′ UTR, or an intron of the mRNA.

In any embodiment, the expression cassette may encode a therapeutic protein, e.g., SCN1A, SNC2A, SNC8A, SCN1B, SCN2B, KV3.1, KV3.2, KV3.3, STXBP1, UBE3A or a transcription factor that modulates, e.g., activates or represses, endogenous expression of any of those proteins. In some embodiments, the therapeutic protein may be ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCNIB, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

In some embodiments, the RNA transcript may comprise a combination of sequences of (i), (ii) and (iii).

Also provided is a vector comprising a cassette as summarized above. The vector may be a plasmid or viral vector, e.g., an adeno-associated virus (AAV) or lentiviral vector.

Also provided is an AAV or lentiviral particle or cell comprising a cassette as summarized above (which may be in single stranded form if it is packaged).

Also provided is an RNA having the sequence characteristics of an RNA encoded by any nucleic acid cassette described herein.

A variety of methods are also provided. In some embodiments, the method may be for expressing a protein. In these embodiments, the method may comprise introducing an expression cassette as summarized above or an mRNA encoded thereby into an organism, wherein the sequence reduces the expression of the protein in DRG and/or liver cells in the organism. In additional embodiments in which the RNA is itself the active agent (i.e., it is a non-coding RNA, e.g., a microRNA, antisense RNA, etc., as described elsewhere herein), the sequence reduces or eliminates the activity of the RNA in DRG and/or liver cells in the organism. In some embodiments, the method may comprise administering an expression cassette or mRNA or non-coding RNA to a patient that has a neural disease or disorder. In these embodiments, the administering may be systemic or local (e.g., administered locally into to the brain or CNS tissue, such as by an intraparenchymal, intrathecal, intra-cisterna magna, intracerebroventricular or intracranial administration method). In these embodiments, the subject to which the expression cassette or mRNA or non-coding RNA is administered may have Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder, for example.

Other features, advantages and embodiments may become apparent in view of the following description.

### BRIEF DESCRIPTION OF THE DRAWINGS

The skilled artisan will understand that the drawings, described below, are for illustration purposes only. The drawings are not intended to limit the scope of the present teachings in any way.

FIG. 1A shows a flow chart of DRG de-targeting regulatory element (RE) library design employed in the Examples.

FIG. 1B is a scatter plot showing the expression levels in brain vs dorsal root ganglion (DRG) for a library of test constructs. Relative transcript abundance (Log₂) for brain (y axis) and DRG tissue (x axis) normalized to AAV library abundance and constitutive control sequences are shown. Relative expression of each sequence element in the library is indicated, compared to the mean of the promoter paired with random control sequences. Each data point represents a unique sequence being screened. Control constructs with randomized sequences in the de-targeting region are indicated with light-shaded circles (inside the solid-line box). The dashed-line region drawn on the scatter plot indicates the brain vs DRG expression pattern used to select candidate DRG de-targeting elements. Examples of candidate DRG de-targeting elements are indicated with dark-shaded circles.

FIG. 2 shows a graph of the log₂ fold-change in EGFP expression in the cortex, DRG, and hippocampus of mice injected with (1) an AAV9 vector in which the EGFP transcript expressed by the vector includes 4 copies of a binding site for hsa-mir-183-3p (4×SEQ ID NO: 4, provided herein as SEQ ID NO: 44) or (2) an AAV9 vector in which the EGFP transcript expressed by the vector includes 4 copies of a binding site for hsa-mir-196b-5p (4×SEQ ID NO: 1, provided herein as SEQ ID NO: 43) as compared to an AAV9 construct with no de-targeting element. Values used for the calculation are mean EGFP-KASH transcript level per μg of total RNA (normalized to VCN/diploid genome).

FIG. 3 shows representative IHC images showing EGFP-KASH expression in cortex/hippocampus (top row) and lumbar DRG (bottom row) tissues harvested from mice injected with AAV9 vectors in which the EGFP transcript expressed by the vector has (1) no de-targeting elements in the de-targeting region (No De-targeting), (2) tetrameric miR-183-3p binding sites in the de-targeting region (4×SEQ ID NO: 4), or (3) tetrameric miR-196b-5p binding sites in the de-targeting region (4×SEQ ID NO: 1). (Scale of the images is provided in the left-most images.)

FIG. 4 shows the average and standard deviation (SD) of percent EGFP positive nuclei from two cortical regions of the brain tissue (top graph) and the four DRG regions (bottom panel) for each treatment arm (n=5; dots on the graph indicate individual animals). An unmanipulated negative control (UNM) was included (no AAV9 vector injection) in each experiment. The treatment arms included AAV9 vectors in which the EGFP transcript expressed by the vector had either no de-targeting element in the de-targeting region (No De-targ) or the indicated de-targeting elements in the de-targeting region (provided as SEQ ID NOs).

FIG. 5 provides examples of representative IHC images showing EGFP-KASH expression in brain and DRG from four mice from FIG. 4: the control mouse injected with an AAV9 vector in which the EGFP transcript expressed by the vector had no de-targeting element in the de-targeting region (No De-targeting Element) and three mice which were each injected with a different AAV9 vector in which the EGFP transcript expressed by the vector had the indicated de-targeting element in the de-targeting region (provided as SEQ ID NOs).

FIGS. 6A and 6B. AAV9 with pan-neuronal promoter and control UTR (control) or candidate DRG de-targeting UTRs (SEQ ID NO: 46, which includes 4 copies of the hsa-mir-10b-5p binding site, and SEQ ID NO: 48, which includes 2 copies of the hsa-mir-196b-5p binding site and 2 copies of the hsa-mir-10b-5p binding site) following ICV administration in mice at P1. FIG. 6A shows the % GFP+ nuclei in DRG (left panel) and in brain (right panel). FIG. 6B shows representative images of mouse samples stained for AAV transgene expression from control vectors with no de-targeting elements in the de-targeting region and vectors in which the de-targeting region of the transcript includes four copies of hsa-mir-10b-5p (SEQ ID NO: 46; same mouse shown in FIG. 6A). Spike-in AAV vector with a myc-tagged mCherry gene under the control of a pan-neuronal promoter was added to controls to assess biodistribution. These experiments show that hsa-mir-10b-5p binding sites in the de-targeting region of an RNA transcript (in this case the 3′UTR) substantially reduce off-target expression in the DRG, while maintaining CNS expression. Individual validation of lead DRG de-targeting elements in mice shows 5 to 16-fold reductions in DRG expression with no reduction brain expression.

FIGS. 7A, 7B, 7C, and 7D. DRG de-targeting element in AAV9 transcript rescues protein overexpression in DRG in mice (FIG. 7A shows IHC and 7B shows intensity values) with no effect on brain expression in mouse (FIG. 7C shows IHC and 7D shows intensity values). The 4× hsa-mir-10b-5p DRG de-targeting element (SEQ ID NO: 46) was evaluated in an AAV9 vector with a neuronal transgene under the control of a pan-neuronal promoter. IHC analysis following ICV administration in mice at P1 showed that the DRG de-targeting element achieved rescue of protein over-expression in DRG (i.e., it reduced it to levels seen in control vehicle injection) without altering expression in brain sections.

FIGS. 8A, 8B, 8C, 8D, 8E, and 8F. Rescue of protein overexpression in NHP DRG tissue with de-targeting element. FIG. 8A shows experimental design of NHP study to evaluate DRG de-targeting element (SEQ ID NO: 46) in NHP. NHP were injected ICV with vehicle (Group 1), an AAV with a pan neuronal promoter driving the expression of an mRNA transcript encoding a neuronal gene (Group 3), or AAV with a pan neuronal promoter driving the expression of an mRNA transcript encoding the neuronal gene that also includes SEQ ID NO: 46 in the 3′UTR (a DRG de-targeting sequence) (Group 5). FIG. 8B shows the molecular analysis of AAV transcript expression between Group 3 (Promoter, denoted “P” in the figure) and Group 5 (P+SEQ ID NO: 46). AAV-driven transcript expression in brain and DRG sections was evaluated by RT-ddPCR analysis using vector-specific primer/probes. Total RNA expression was normalized to AAV genome copy number in each section and represented as a fold change to the promoter only condition. No difference in AAV-driven transgene expression in forebrain and midbrain was detected while a >10-fold reduction in AAV-mediated transcript expression was seen across DRG sections (cervical, thoracic, lumbar, and sacral were analyzed). FIG. 8C shows vector biodistribution in cervical (C), thoracic (T), lumbar (L), and sacral(S) spinal cord DRG segments. Expression in DRG is found primarily in the sacral DRG tissue. Protein expression driven by the transgene (FIG. 8D) and endogenously (FIG. 8E) by MSD-ELISA (using reagents specific for either the transgene-encoded protein or the endogenous protein) is shown in cervical (C), thoracic (T), lumbar (L), and sacral(S) spinal cord DRG segments. This figure shows that the presence of DRG de-targeting element SEQ ID NO: 46 in an mRNA transcript (which includes four copies of the hsa-mir-10b-5p binding site) reduces expression of the transgene encoded protein but does not significantly impact expression of the endogenous gene. IHC staining for both the transgene encoded and endogenous protein in sacral(S) DRG section is show in FIG. 8F. This figure shows that without the DRG de-targeting element in the transcript, protein expression was significantly higher in the sacral DRG tissue (center panel) as compared to the endogenous levels (left panel). Inclusion of binding sites for hsa-mir-10b-5p, a DRG de-targeting element, reduced the level of the protein expression to endogenous levels (right panel).

FIG. 9 is a scatter plot showing the log₂ change in brain versus liver activity for a screen of liver de-targeting elements. Constructs selected as liver de-targeting elements are shown in dark squares. Control sequences (not liver de-targeting) are shown in light squares.

FIG. 10 is a graph showing results of an ELISA assay comparing a construct in which the transcript expressed therefrom has no de-targeting element (No element Control) to constructs in which the transcript expressed therefrom includes the indicated liver de-targeting element. SEQ ID NO: 113 is a positive control sequence.

FIGS. 11A and 11B shows representative images showing the in vivo expression of various constructs in brain and liver using a CNS specific promoter. FIG. 11A shows representative images of brain expression and liver expression of a myc-tagged transgene from (i) a mouse treated with a control vector that expresses a transcript (encoding the myc-tagged protein) without a de-targeting element (top panels; No De-targ) and (ii) a mouse treated with a vector that expresses a transcript (encoding the myc-tagged protein) containing SEQ ID NO: 66 in its de-targeting region (lower panels). The AAV vector in FIG. 11A was delivered via tail vein injection. FIG. 11B shows representative images of brain expression and liver expression of a myc-tagged transgene from (i) a mouse injected ICM with a control vector that expresses a transcript (encoding the myc-tagged protein), under the control of a pan-neuronal promoter, without a de-targeting element (top panels; No De-targ), (ii) a mouse treated with a vector that expresses a transcript (encoding the myc-tagged protein), under the control of a pan-neuronal promoter, containing SEQ ID NO: 110 in its de-targeting region (middle panels; SEQ ID NO: 110 includes 2 copies of the hsa-mir-19a-3p binding site (SEQ ID NO: 65), 2 copies of the hsa-mir-1258-5p binding site (SEQ ID NO: 58), and 2 copies of the hsa-mir-17-5p binding site (SEQ ID NO: 60)), and (iii) a mouse treated with a vector that expresses a transcript (encoding the myc-tagged protein), under the control of a pan-neuronal promoter, containing SEQ ID NO: 112 in its de-targeting region (lower panels; SEQ ID NO: 112 includes 3 copies of the hsa-mir-122-3p binding site (SEQ ID NO: 62)).

FIGS. 12A and 12B show the fold-change in myc-positive cells present in brain tissue (FIG. 12A) and liver tissue (FIG. 12B) from the mice in FIG. 11B as well as a mouse injected ICM with an AAV vector that expresses the myc-tagged protein under the control of a ubiquitous chicken beta actin promoter (“CBA”) and without a de-targeting element. The liver de-targeting activity of SEQ ID NO: 110 (“110”) and SEQ ID NO: 112 (“112”) were compared to expression from the CNS promoter (CNS) as well as a ubiquitous chicken beta actin promoter (“CBA”) (*** and **** indicate significant differences in expression).

FIG. 13 is a graph showing relative expression in liver (log₂ of fold change) for several different liver de-targeting elements in NHPs. Data for two different NHP animals (Animal 1 and Animal 2) and the average are shown. SEQ ID NO: 55 is a random control.

FIG. 14 shows schematic of the design and construction of a combinatorial AAV library using select DRG de-targeting and liver de-targeting elements of the present disclosure. The AAV vector structure is shown at the top and includes a promoter operably linked to a transgene that includes a 3′UTR with a de-targeting region that has three positions (Positions 1, 2, and 3) into which a de-targeting element or control/benchmark element is inserted. All permutations of the elements were present in the AAV library. The SEQ ID NOs of the selected DRG, Liver, and control/benchmark elements are shown in the tables, both in DNA and RNA form (DNA is the sequence in the AAV vector; RNA is the sequence in the mRNA transcript expressed from the vector).

FIG. 15 shows scatter plots of the expression pattern of individual vectors in the AAV library in mouse tissues. The top panel compares the log₂ brain expression activity (y axis) versus the log₂ DRG expression activity (x axis) and the bottom panel the log₂ brain expression activity (y axis) versus the log₂ liver expression activity (x axis). In the top panel, the highlighted vector points include one or more of the top DRG de-targeting elements. In the bottom panel, the highlighted vector points include one or more of the top liver de-targeting elements. In both panels, the dark vector points are those with only neutral control sequences. This data shows that DRG and liver de-targeting elements are very effective at reducing transgene expression in their respective tissues while maintaining expression in brain tissue.

FIG. 16 shows the effect of individual elements in the AAV combinatorial library on transgene expression in DRG, liver, and Forebrain tissue in mice injected with the combinatorial library. These values were determined by recovering the transcribed library from DRG, liver, and brain tissue sections using amplicon sequencing of RNA/AAV DNA and NGS quantification for differential expression. Regression-based models of tissue expression showed top contributing elements across thousands of data points/instances. The data is presented as log 2 fold-change in the specified tissue in the presence and absence of the de-targeting element identified on the x axis by SEQ ID NO. Each SEQ ID NO represents the RNA sequence present in the transcript in the de-targeting region of the expressed transcript except for SEQ ID NO: 55, which is the DNA sequence of the random control element in the AAV vector library. SEQ ID NO: 119 is the DRG de-targeting benchmark and SEQ ID NO: 113 is the liver de-targeting benchmark. Modelling results identified top 3′UTR sequence elements with selective DRG and/or liver de-targeting while maintaining expression in brain.

FIG. 17. Combined de-targeting sequence elements show a range of de-targeting in both liver and DRG (scatter plot, top panel). Candidate combinatorial de-targeting regions were identified that exhibit strong de-targeting in both DRG and liver (dark highlighted circles in scatter plot). These candidate combinatorial de-targeting regions include at least one DRG and at least one liver de-targeting element in the de-targeting region of the 3′UTR. Select combinatorial de-targeting elements were shown to have no negative effect on transgene expression in brain tissue (bottom graph). This graph shows the log₂ fold-change in the specified tissue in the presence and absence of the combinatorial de-targeting element identified on the x axis by the three SEQ ID NOs. The three SEQ ID NOs represents the RNA sequences present in the transcript in the de-targeting region of the expressed transcript except for SEQ ID NOs: 55 and 56 in the last column, which are the DNA sequences of the random control elements in the AAV vector library.

FIG. 18. Combinatorial analysis for multiplex validation of DRG de-targeting sequences in NHP. NHP (n=2) were dosed via intracerebroventricular (ICV) injection with a combinatorial AAV library designed as shown in FIG. 14. DRG de-targeting coefficients were determined from high-transduction samples as described for FIG. 14 from each tissue: DRG (sacral sections) and brain (hippocampus sections). FIG. 18 shows regression-based models of differential expression to describe the de-targeting contribution of each element across all points/instances in the recovered library. The SEQ ID NO on the x axis represents the RNA sequence present in the transcript in the de-targeting region of the expressed transcript except for SEQ ID NOs: 55 and 56 in the last two columns, which are the DNA sequences of the random control elements in the AAV vector library.

FIG. 19. Correlation is shown on the effect of the elements in the combinatorial AAV library on expression in brain, DRG and liver tissues between mouse and NHP. As shown in FIG. 19, the Pearson's correlation between the mouse and NHP expression coefficients for each of the de-targeting elements was 0.86 for brain expression, 0.91 for liver expression, and 0.79 for DRG expression.

### DEFINITIONS

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “AAV” is an abbreviation for adeno-associated virus and may be used to refer to the virus itself or a derivative thereof. The term covers all serotypes, subtypes, and both naturally occurring and recombinant forms, except where required otherwise. The abbreviation “rAAV” refers to recombinant adeno-associated virus. The term “AAV” includes all serotypes of AAV, including AAV1, AAV2, AAV3, AAV4, AAV5, AAV6, AAV7, AAV8, AAV9, AAV9.47, AAV9 (hul4), AAV10, AAV11, AAV 12, AAV13, AAVrh8, AAVrh10, AAV-DJ, and AAV-DJ8, and hybrids thereof (i.e., chimeric AAV vectors), as well as avian AAV, bovine AAV, canine AAV, equine AAV, primate AAV, non-primate AAV, and ovine AAV. The genomic sequences of various serotypes of AAV, as well as the sequences of the native terminal repeats (TRs), Rep proteins, and capsid subunits are known in the art. Such sequences may be found in the literature or in public databases such as GenBank. A “rAAV vector” as used herein refers to an AAV vector comprising a polynucleotide sequence not of AAV origin (i.e., a polynucleotide heterologous to AAV), typically a sequence of interest for the genetic transformation of a cell. In general, the heterologous polynucleotide is flanked by at least one, and generally by two, AAV inverted terminal repeat sequences (ITRs). An rAAV vector may either be single-stranded (ssAAV) or self-complementary (scAAV). See, e.g., Raj et al., Expert Rev Hematol. 2011 October; 4 (5): 539-549. An “AAV virus” or “AAV viral particle” refers to a viral particle composed of at least one AAV capsid protein and an encapsidated polynucleotide rAAV vector. If the particle comprises a heterologous polynucleotide (i.e., a polynucleotide other than a wild-type AAV genome such as a transgene to be delivered to a mammalian cell), it is typically referred to as an “rAAV viral particle” or simply an “rAAV particle”. AAVs may comprise genome components and capsids from multiple serotypes (e.g., pseudotyped vectors). For example, an AAV may comprise the genome of serotype 2 (e.g., ITRs) packaged in the capsid from serotype 5 or serotype 9. Pseudotyped vectors may demonstrate improved transduction efficiency as well as altered tropism. In some cases, an AAV serotype that can cross the blood brain barrier or infect cells of the CNS is preferred. In some aspects, the recombinant AAV vector is AAV1, AAV8, AAV9, AAVDJ, or chimeric AAV comprising features of two or more of these serotypes. In various embodiments, the AAV vector is an AAV9 vector or an scAAV9 vector. In certain embodiments, the AAV vector is an AAV9 vector or an scAAV9 vector and comprises a heterologous nucleic acid flanked by ITRs from a AAV serotype other than AAV9. In certain embodiments, the AAV vector is an AAV9 vector or an scAAV9 vector and comprises a heterologous nucleic acid flanked by AAV serotype 2 ITRs (i.e., ITR2).

The term “about” or “approximately” means within an acceptable error range for the particular value as determined by one of ordinary skill in the art, which will depend in part on how the value is measured or determined, i.e., the limitations of the measurement system. For example, “about” can mean within one or more than one standard deviation, per the practice in the art. Alternatively, “about” can mean a range of up to 20%, up to 15%, up to 10%, up to 5%, or up to 1% of a given value.

In any of the embodiments described herein, “comprising” may be replaced with “consisting essentially of” or “consisting of.” For example, an embodiment in which a particular element is included using the open-ended term “comprising” encompasses embodiments in which the element is included using the more restrictive terms “consisting essentially of” or “consisting of”.

The terms “determining”, “measuring”, “evaluating”, “assessing”, “assaying”, “analyzing”, and their grammatical equivalents can be used interchangeably herein to refer to any form of measurement and include determining if an element is present or not (for example, detection). These terms can include both quantitative and/or qualitative determinations. Assessing may be relative or absolute.

The term “expression” refers to the process by which a nucleic acid sequence or a polynucleotide is transcribed from a DNA template (such as into mRNA or non-coding RNA transcript) and/or the process by which a transcribed mRNA is subsequently translated into peptides, polypeptides, or proteins. Transcripts and encoded polypeptides may be collectively referred to as “gene product.” If the polynucleotide includes introns or splice sites, e.g., is derived from genomic DNA, expression may include splicing of the mRNA in a eukaryotic cell.

An “expression cassette” refers to a nucleic molecule comprising one or more regulatory elements operably linked to a sequence to be expresses as an RNA transcript, including mRNA (i.e., an RNA molecule that includes a coding sequence (e.g., a gene or genes) for expression of a protein) or a non-coding RNA.

A “transgene” refers to a portion of a nucleic acid cassette that is designed to be expressed in a cell. In some embodiments, a transgene encodes an RNA transcript, e.g., an mRNA or a non-coding RNA, e.g., an antisense RNA. In some embodiments, a transgene of the present disclosure encodes a therapeutic cargo, e.g., a therapeutic protein or a therapeutic RNA, and also includes one or more DRG and/or liver de-targeting sequences/elements to reduce expression of the transgene in DRG and/or liver cells.

An “RNA transcript” refers to an RNA molecule that is transcribed from a template, e.g., an RNA molecule transcribed from an expression cassette as described herein. An RNA transcript that is expressed from an expression cassette described herein can be in any desired form, including an mRNA encoding a polypeptide/protein or an RNA that exerts it desired function without being used as a template for protein expression, also referred to as non-coding RNA (ncRNA). Examples of ncRNA include, but are not limited to: microRNA (miRNA or miR), primary microRNA (pri-miRNA or pri-miR) pre-microRNA (pre-miRNA or pre-miR), small nuclear RNAs (snRNA), small nucleolar RNA (snoRNA), piwi-interacting RNA (piRNA), antisense RNA (asRNA), transfer RNA (tRNA), long non-coding RNA (lncRNA), short interfering RNA (siRNA), short hairpin RNA (shRNA), ribozyme, CRISPR guide RNA (gRNA), and the like.

The term “effective amount” or “therapeutically effective amount” refers to that amount of a composition described herein that is sufficient to affect the intended application, including but not limited to disease treatment, as defined below. The therapeutically effective amount may vary depending upon the intended treatment application (in a cell or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in a target cell. The specific dose will vary depending on the particular composition chosen, the dosing regimen to be followed, whether it is administered in combination with other compounds, timing of administration, the tissue to which it is administered, and the physical delivery system in which it is carried.

A “fragment” of a nucleotide or peptide sequence is meant to refer to a sequence that is less than that believed to be the “full-length” sequence.

A “functional fragment” of a DNA, RNA, or protein sequence refers to a biologically active fragment of the sequence that is shorter than the full-length or reference DNA, RNA, or protein sequence, but which retains at least one biological activity (either functional or structural) that is substantially similar to a biological activity of the full-length or reference DNA, RNA, or protein sequence. For example, a “functional fragment” may be a fragment of a sequence disclosed herein that reduces expression of the transgene to which it is operably linked in DRG and/or liver cells.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that have the same function or biological activity as screened or selected for in the originally transformed cell are included herein.

The term “human derived” as used herein refers to sequences that are found in a human genome (or a human genome build), or sequences homologous thereto. A homologous sequence may be a sequence which has a region with at least 80% sequence identity (e.g., as measured by BLAST) as compared to a region of the human genome. For example, a sequence that has at least 80%, at least 85%, at least 90%, at least 91%, at least 92%, at least 93%, at least 94%, at least 95%, at least 96%, at least 97%, at least 98%, at least 99% or 100% identity to a human sequence is deemed human derived. In some cases, a regulatory element contains a human derived sequence and a non-human derived sequence such that overall the regulatory element has low sequence identity to the human genome, while a part of the regulatory element has 100% sequence identity (or local sequence identity) to a sequence in the human genome.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

The term “in vivo” refers to an event that takes place in a subject's body.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally, at a chromosomal location that is different from its natural chromosomal location, or contains only coding sequences.

As used herein, “operably linked”, “operable linkage”, “operatively linked”, or grammatical equivalents thereof refer to juxtaposition of genetic elements, e.g., a promoter, an enhancer, a polyadenylation sequence, etc., wherein the elements are in a relationship permitting them to operate in the expected manner. For instance, a regulatory element, which can comprise promoter and/or enhancer sequences, is operatively linked to a coding region if the regulatory element helps initiate transcription of the coding sequence. There may be intervening residues between the regulatory element and coding region so long as this functional relationship is maintained.

A “pharmaceutically acceptable carrier” refers to an ingredient in a pharmaceutical formulation or composition, other than an active ingredient, which is nontoxic to a subject. A pharmaceutically acceptable carrier includes, but is not limited to, a buffer, excipient, stabilizer, or preservative.

The terms “pharmaceutical formulation” or “pharmaceutical composition” refer to a preparation which is in such form as to permit the biological activity of an active ingredient contained therein to be effective, and which contains no additional components which are unacceptably toxic to a subject to which the formulation would be administered.

The term “regulatory element” refers to a nucleic acid sequence or genetic element which is capable of influencing (e.g., increasing, decreasing, or modulating) expression of an operably linked sequence, such as a gene, a coding sequence, or an RNA (e.g., an mRNA or ncRNA). Regulatory elements include, but are not limited to, promoter, enhancer, repressor, silencer, insulator sequences, an intron, UTR, an inverted terminal repeat (ITR) sequence, a long terminal repeat sequence (LTR), a stability element, a miRNA target site, a posttranslational response element, or a polyA sequence, or a combination thereof. Regulatory elements can function at the DNA and/or the RNA level, e.g., by modulating gene expression at the transcriptional phase, post-transcriptional phase, or at the translational phase of gene expression; by modulating the level of translation (e.g., stability elements that stabilize mRNA for translation), RNA cleavage, RNA splicing, and/or transcriptional termination; by recruiting transcriptional factors to a coding region that increase gene expression; by increasing the rate at which RNA transcripts are produced, increasing the stability of RNA produced, and/or increasing the rate of protein synthesis from RNA transcripts; and/or by preventing RNA degradation and/or increasing its stability to facilitate protein synthesis. In an exemplary embodiment, a regulatory element refers to an enhancer, repressor, promoter, or a combination thereof, particularly an enhancer plus promoter combination or a repressor plus promoter combination. In exemplary embodiments, the regulatory element is derived from a human sequence.

In general, “sequence identity” or “sequence homology”, which can be used interchangeably, refer to an exact nucleotide-to-nucleotide or amino acid-to-amino acid correspondence of two polynucleotides or polypeptide sequences, respectively. Two or more sequences (polynucleotide or amino acid) can be compared by determining their “percent identity”, also referred to as “percent homology”. The percent identity to a reference sequence (e.g., nucleic acid or amino acid sequence) may be calculated as the number of exact matches between two optimally aligned sequences divided by the length of the reference sequence and multiplied by 100. Conservative substitutions are not considered as matches when determining the number of matches for sequence identity. It will be appreciated that where the length of a first sequence (A) is not equal to the length of a second sequence (B), the percent identity of A:B sequence will be different than the percent identity of B: A sequence. Sequence alignments, such as for the purpose of assessing percent identity, may be performed by any suitable alignment algorithm or program, including but not limited to the Needleman-Wunsch algorithm, the BLAST algorithm, the Smith-Waterman algorithm (see, e.g., the EMBOSS Water aligner), and Clustal Omega alignment program (F. Sievers et al., Mol Sys Biol. 7:539 (2011)). Optimal alignment may be assessed using any suitable parameters of a chosen algorithm, including default parameters. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997).

The terms “subject” and “individual” are used interchangeably herein to refer to a vertebrate, preferably a mammal, more preferably a human. The methods described herein can be useful in human therapeutics, veterinary applications, and/or preclinical studies in animal models of a disease or condition.

As used herein, the terms “treat”, “treatment”, “therapy” and the like refer to obtaining a desired pharmacologic and/or physiologic effect, including, but not limited to, alleviating, delaying or slowing progression, reducing effects or symptoms, preventing onset, preventing reoccurrence, inhibiting, ameliorating onset of a diseases or disorder, obtaining a beneficial or desired result with respect to a disease, disorder, or medical condition, such as a therapeutic benefit and/or a prophylactic benefit. “Treatment,” as used herein, covers any treatment of a disease in a mammal, particularly in a human, and includes: (a) preventing the disease from occurring in a subject which may be predisposed to the disease or at risk of acquiring the disease but has not yet been diagnosed as having it; (b) inhibiting the disease, i.e., arresting its development; and (c) relieving the disease, i.e., causing regression of the disease. A therapeutic benefit includes eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In some cases, for prophylactic benefit, the compositions are administered to a subject at risk of developing a particular disease, or to a subject reporting one or more of the physiological symptoms of a disease, even though a diagnosis of this disease may not have been made. The methods of the present disclosure may be used with any mammal. In some cases, the treatment can result in a decrease or cessation of symptoms. A prophylactic effect includes delaying or eliminating the appearance of a disease or condition, delaying or eliminating the onset of one or more symptoms of a disease or condition, slowing, halting, or reversing the progression of a disease or condition, or any combination thereof.

A “variant” of a nucleotide sequence refers to a sequence having a genetic alteration or a mutation as compared to the most common wild-type DNA sequence (e.g., cDNA or a sequence referenced by its GenBank accession number) or a specified reference sequence. A variant can be shorter than the reference sequence and/or have one or more mutations relative to the reference sequence. In some cases, a variant may have a nucleotide sequence that is at least 80% identical, at least 90% identical or at least 95% identical to a reference sequence.

A “vector” as used herein refers to a nucleic acid molecule that can be used to mediate delivery of another nucleic acid molecule to which it is linked into a cell where it can be replicated or expressed. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Other examples of vectors include plasmids and viral vectors.

As used herein a “target cell” is generally a cell in which expression of RNA or protein product of the nucleic acid cassette is desired. A non-target cell is a cell in which expression of the RNA or protein product of the nucleic acid is not desired. As used herein “de-targeting” generally refers to decreasing the expression in a non-target cell.

Unless otherwise indicated, all terms used herein have the same meaning as they would to one skilled in the art and the practice of the present invention will employ, conventional techniques of molecular biology, microbiology, and recombinant DNA technology, which are within the knowledge of those of skill of the art.

### DETAILED DESCRIPTION

Before the present invention is further described, it is to be understood that this invention is not limited to particular embodiments described, and as such may vary. It is also to be understood that the terminology used herein is for the purpose of describing particular embodiments only, and is not intended to be limiting, since the scope of the present invention will be limited only by the appended claims.

The upper and lower limits of ranges may independently be included in the ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. Thus, for example, reference to “a protein” includes a plurality of such proteins and reference to “the nucleic acid” includes reference to one or more nucleic acids and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Aspects of the present disclosure provide nucleic acid molecules that include one or more DRG de-targeting elements, one or more liver de-targeting elements, or combinations of both. In certain embodiments, the nucleic acid molecule is an RNA molecule that further includes a heterologous RNA sequence, e.g., an RNA sequence that encodes a protein or a non-coding RNA (ncRNA). In some of these embodiments, the heterologous RNA is a therapeutic RNA, e.g., encoding a therapeutic protein or a ncRNA having a desired therapeutic function. In some embodiments, the nucleic acid molecule is a DNA molecule that further includes a heterologous DNA sequence, e.g., a DNA sequence that can be used as a template to generate an RNA transcript containing the one or more de-targeting elements and a heterologous RNA sequence. In some embodiments, the DNA molecule is a nucleic acid cassette designed to express an RNA transcript that includes the one or more de-targeting elements and a heterologous RNA sequence. As described in detail herein, and summarized above, the presence of one or more DRG de-targeting elements in an RNA molecule with a heterologous RNA sequence reduces the activity of the heterologous RNA sequence in DRG cells, e.g., DRG cells of a subject, compared to an RNA molecule containing the heterologous RNA sequence without the one or more DRG de-targeting elements. Similarly, the presence of one or more liver de-targeting elements in an RNA molecule with a heterologous RNA sequence reduces the activity of the heterologous RNA sequence in liver cells, e.g., liver cells of a subject, compared to an RNA molecule containing the heterologous RNA sequence without the one or more DRG de-targeting elements. Further, the presence of one or more DRG de-targeting elements and one or more liver de-targeting elements in an RNA molecule with a heterologous RNA sequence reduces the activity of the heterologous RNA sequence in DRG cells and in liver cells, e.g., DRG cells and liver cells of a subject, compared to an RNA molecule containing the heterologous RNA sequence without the one or more DRG de-targeting elements and liver de-targeting elements.

Aspects of the present disclosure provide nucleic acid molecules that include one or more regions that hybridize under physiologic conditions (e.g., in a cell of a subject) to hsa-mir-196b-5p, hsa-mir-10b-5p, hsa-mir-24-2-5p, hsa-mir-183-3p, hsa-mir-196a-5p, hsa-mir-494-3p, or any combination thereof (each of which are de-targeting elements of the present disclosure). These hybridizing regions can be designated as binding sites for a particular miRNA. In certain embodiments, the nucleic acid molecule is an RNA molecule that further includes a heterologous RNA sequence, e.g., an RNA sequence that encodes a protein or a non-coding RNA (ncRNA). In some of these embodiments, the heterologous RNA is a therapeutic RNA, e.g., encoding a therapeutic protein or a ncRNA having a desired therapeutic function. In some embodiments, the nucleic acid molecule is a DNA molecule that further includes a heterologous DNA sequence, e.g., a DNA sequence that can be used as a template to generate an RNA transcript containing the one or more binding sites. In some embodiments, the DNA molecule is a nucleic acid cassette designed to express an RNA transcript that includes the one or more de-targeting elements and a heterologous RNA sequence. As described in detail herein, and summarized above, the presence of one or more regions that hybridize under physiologic conditions to hsa-mir-196b-5p, hsa-mir-10b-5p, hsa-mir-24-2-5p, hsa-mir-183-3p, hsa-mir-196a-5p, hsa-mir-494-3p, or any combination thereof, in an RNA molecule with a heterologous RNA sequence reduces the activity of the heterologous RNA sequence in DRG cells, e.g., DRG cells of a subject, compared to an RNA molecule containing the heterologous RNA sequence without the one or more hybridization regions. Aspects of the present disclosure thus include nucleic acid molecules that include one or more regions that hybridize under physiologic conditions to hsa-mir-196b-5p (SEQ ID NO: 21). Aspects of the present disclosure thus include nucleic acid molecules that include one or more regions that hybridize under physiologic conditions to hsa-mir-10b-5p (SEQ ID NO: 22). Aspects of the present disclosure thus include nucleic acid molecules that include one or more regions that hybridize under physiologic conditions to hsa-mir-24-2-5p (SEQ ID NO: 23). Aspects of the present disclosure thus include nucleic acid molecules that include one or more regions that hybridize under physiologic conditions to hsa-mir-183-3p (SEQ ID NO: 24). Aspects of the present disclosure thus include nucleic acid molecules that include one or more regions that hybridize under physiologic conditions to hsa-mir-196a-5p (SEQ ID NO: 25). Aspects of the present disclosure thus include nucleic acid molecules that include one or more regions that hybridize under physiologic conditions to hsa-mir-494-3p (SEQ ID NO: 26).

In certain aspects, the present disclosure provides nucleic acid molecules that include one or more binding sites for hsa-mir-196b-5p, hsa-mir-10b-5p, hsa-mir-24-2-5p, hsa-mir-183-3p, hsa-mir-196a-5p, hsa-mir-494-3p, or any combination thereof (each of which are de-targeting elements of the present disclosure). In certain embodiments, the nucleic acid molecule is an RNA molecule that further includes a heterologous RNA sequence, e.g., an RNA sequence that encodes a protein or a non-coding RNA (ncRNA). In some of these embodiments, the heterologous RNA is a therapeutic RNA, e.g., encoding a therapeutic protein or a ncRNA having a desired therapeutic function. In some embodiments, the nucleic acid molecule is a DNA molecule that further includes a heterologous DNA sequence, e.g., a DNA sequence that can be used as a template to generate an RNA transcript containing the one or more binding sites. In some embodiments, the DNA molecule is a nucleic acid cassette designed to express an RNA transcript that includes the one or more de-targeting elements and a heterologous RNA sequence. As described in detail herein, and summarized above, the presence of one or more binding sites for hsa-mir-196b-5p, hsa-mir-10b-5p, hsa-mir-24-2-5p, hsa-mir-183-3p, hsa-mir-196a-5p, hsa-mir-494-3p, or any combination thereof, in an RNA molecule with a heterologous RNA sequence reduces the activity of the heterologous RNA sequence in DRG cells, e.g., DRG cells of a subject, compared to an RNA molecule containing the heterologous RNA sequence without the one or more binding sites. Aspects of the present disclosure thus include nucleic acid molecules that include one or more binding sites for hsa-mir-196b-5p. Aspects of the present disclosure thus include nucleic acid molecules that include one or more binding sites for hsa-mir-10b-5p. Aspects of the present disclosure thus include nucleic acid molecules that include one or more binding sites for hsa-mir-24-2-5p. Aspects of the present disclosure thus include nucleic acid molecules that include one or more binding sites for hsa-mir-183-3p. Aspects of the present disclosure thus include nucleic acid molecules that include one or more binding sites for hsa-mir-196a-5p. Aspects of the present disclosure thus include nucleic acid molecules that include one or more binding sites for hsa-mir-494-3p.

Aspects of the present disclosure provide nucleic acid molecules that include one or more regions that hybridize under physiologic conditions to hsa-mir-19a-3p (SEQ ID NO: 95) (which is a de-targeting element of the present disclosure). Aspects of the present disclosure provide nucleic acid molecules that include one or more binding sites for hsa-mir-19a-3p. In certain embodiments, the nucleic acid molecule is an RNA molecule that further includes a heterologous RNA sequence, e.g., an RNA sequence that encodes a protein or a non-coding RNA (ncRNA). In some of these embodiments, the heterologous RNA is a therapeutic RNA, e.g., encoding a therapeutic protein or a ncRNA having a desired therapeutic function. In some embodiments, the nucleic acid molecule is a DNA molecule that further includes a heterologous DNA sequence, e.g., a DNA sequence that can be used as a template to generate an RNA transcript containing the one or more hsa-mir-19a-3p binding sites. In some embodiments, the DNA molecule is a nucleic acid cassette designed to express an RNA transcript that includes the one or more hsa-mir-19a-3p binding sites and a heterologous RNA sequence. As described in detail herein, and summarized above, the presence of one or more hsa-mir-19a-3p binding sites in an RNA molecule with a heterologous RNA sequence reduces the activity of the heterologous RNA sequence in liver cells, e.g., liver cells of a subject, compared to an RNA molecule containing the heterologous RNA sequence without the one or more hsa-mir-19a-3p binding sites.

In some embodiments, a nucleic acid molecule of the present disclosure includes one or more of the hybridizing regions/miRNA binding sites described above, in any combination, and one or more additional hybridizing region/miRNA binding sites, e.g., that de-target a cell or tissue of interest, e.g., as described herein. In some embodiments, a nucleic acid molecule of the present disclosure includes one or more of the hybridizing regions and/or miRNA binding sites described above, in any combination, and one or more additional de-targeting element, e.g., for DRG and/or liver, as described herein. No limitation in this regard is intended.

As summarized above, this disclosure describes a nucleic acid cassette comprising a transgene encoding an RNA, wherein the RNA comprises a sequence of (i) any of SEQ ID NOs. 1-10 and 43-48, (ii) a functional fragment thereof, or (iii) a sequence at least 80% identical to (i) or (ii), or any combination thereof. The transgene may encode a protein coding mRNA, or a non-coding RNA such as a pri-miRNA, pre-miRNA, or a miRNA, an antisense RNA, a short non-coding RNA, a long non-coding RNA, a snoRNA, a snRNA, a tRNA or an rRNA. In some cases, the nucleic acid cassette comprises a transgene encoding an mRNA, wherein the mRNA comprises a sequence of (i) any of SEQ ID NOs. 1-10 and 43-48, (ii) a functional fragment thereof, or (iii) a sequence at least 80% identical to (i) or (ii), or any combination thereof. These sequences reduce the expression of the transgene in dorsal root ganglion cells (DRG) relative to target cells (such as neural cells, e.g., neurons) and as such, may be employed in a variety of gene therapy strategies that target cells that are not in the DRG. Reducing expression of the transgene in DRG cells relative to target cells means that the reduction in transgene expression driven by the DRG de-targeting sequences disclosed herein is greater in DRG cells than in the target cells. As such, while reduced transgene expression in target cells may be observed in certain embodiments, it is less than that observed in DRG cells. This reduction in expression in the DRG can reduce or eliminate DRG toxicity and/or axonopathy in a subject receiving a gene therapy targeted to a non-DRG cell or tissue, e.g., neural cells, e.g., neurons, thereby improving its safety profile.

The present disclosure further provides an RNA molecule having the sequence characteristics of an RNA encoded by any of the nucleic acid cassettes described herein. In certain embodiments, the RNA is modified to increase its stability and/or activity when administered to a subject, e.g., as a pharmaceutical composition. RNA compositions find use in a variety of therapeutic modalities delivered using a wide range of viral and non-viral delivery systems, the latter including polymeric materials, ionizable lipids, cell-penetrating and zwitterionic lipids, nanoparticles, and dendrimers (see, e.g., Kowalski et al., “Delivering the Messenger: Advances in Technologies for Therapeutic mRNA Delivery” Molecular Therapy 2019 v.27 (4) pp. 710-728 and Paunovska et al. “Drug delivery systems for RNA therapeutics” Nature Reviews genetics 2022) v23 pp. 265-280).

The RNA encoded by the transgene of the nucleic acid cassette (e.g., an mRNA or ncRNA) may contain any combination of two, three, four or five or more of the sequences. For example, the RNA comprising a sequence of (i) any of SEQ ID NOs. 1-10 and 43-48, (ii) a variant or functional fragment thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), may further comprise a second sequence of (i), (ii), or (iii), a third sequence of (i), (ii), or (iii), a fourth sequence of (i), (ii), or (iii), and/or five or more sequences of (i), (ii), or (iii). In any embodiment, the nucleic acid cassette may comprise two or more copies (e.g., two, three, four, five, or more than five copies) of a sequence of (i), (ii), or (iii).

In certain embodiments, the RNA encoded by the transgene of the nucleic acid cassette may contain (i) any of SEQ ID NOs. 1-10 and 43-48, (ii) a variant or functional fragment thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii).

In any embodiment, the sequence can be in a 3′ UTR, a 5′ UTR or an intron of a mRNA, for example. If the mRNA contains more than one of the sequences, then the sequences may be in different parts of the mRNA. In many embodiments, however, the sequences are in the 3′ UTR of the mRNA. In these embodiments, the sequence of (i) any of SE SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), may be located in one or more of: a 3′ UTR region of the mRNA, a 5′ UTR of the mRNA or an intron of the mRNA.

Any nucleic acid described herein may be non-naturally occurring, where the term “non-naturally occurring” refers to a composition that does not exist in nature. In some aspects, a non-naturally occurring nucleic acid contains a contiguous, uninterrupted sequence of nucleotides that is not found in nature, i.e., it is different to any nucleic acid in its natural state (i.e., having less than 100% sequence identity to a naturally occurring nucleic acid sequence). Two regions of a non-naturally occurring nucleic acid are “heterologous” to one another if they are derived from separate genomic regions that are not found as a contiguous, uninterrupted nucleic acid sequence in their natural state. For example, in some embodiments, a nucleic acid cassette may be composed of a promoter, a coding sequence, a sequence encoding a DRG de-targeting element (as disclosed herein), and a terminator, where the promoter, the coding sequence, the sequence encoding the DRG de-targeting element, and the terminator are in operable linkage. In these embodiments, at least one of these elements is heterologous to another one of the elements. For example, the coding sequence may be heterologous to the sequence encoding the DRG de-targeting element, meaning that the sequence encoding the DRG de-targeting element is not operably linked in an identical manner to that coding sequence in a wild type cell. In any embodiment, the nucleic acid cassette may additionally comprise an enhancer.

In any embodiment, the RNA encoded by the transgene of the nucleic acid cassette may comprise a functional fragment of any of SEQ ID NOs. 1-10 and 43-48, where the functional fragment reduces expression of the RNA to which it is operably linked in the DRG. In addition, the functional fragment may or may not contain mismatches relative to SEQ ID NOS. 1-8, e.g., one, two, three, four, or more mismatches.

In certain embodiments, a functional fragment comprises any contiguous stretch of nucleotides in SEQ ID NO: 1 of at least 10, at least 11, at least 12, at least 13, at least 14, at least 15, at least 16, at least 17, at least 18, at least 19, at least 20, or at least 21 nucleotides in length. In certain embodiments, a functional fragment of SEQ ID NO: 1 comprises one, two, three, or four mismatches as compared to the corresponding contiguous stretch of nucleotides in SEQ ID NO: 1. A functional fragment may start at any nucleotide in SEQ ID NO: 1 that allows for its full representation in SEQ ID NO: 1.

In certain embodiments, a functional fragment comprises any contiguous stretch of nucleotides in SEQ ID NO: 2 of at least 10, at least 11, at least 12, at least 13, at least 14, at least 15, at least 16, at least 17, at least 18, at least 19, at least 20, at least 21, or at least 22 nucleotides in length. In certain embodiments, a functional fragment of SEQ ID NO: 2 comprises one, two, three, or four mismatches as compared to the corresponding contiguous stretch of nucleotides in SEQ ID NO: 2. A functional fragment may start at any nucleotide in SEQ ID NO: 2 that allows for its full representation in SEQ ID NO: 2.

In certain embodiments, a functional fragment comprises any contiguous stretch of nucleotides in SEQ ID NO: 3 of at least 10, at least 11, at least 12, at least 13, at least 14, at least 15, at least 16, at least 17, at least 18, at least 19, at least 20, or at least 21 nucleotides in length. In certain embodiments, a functional fragment of SEQ ID NO: 3 comprises one, two, three, or four mismatches as compared to the corresponding contiguous stretch of nucleotides in SEQ ID NO: 3. A functional fragment may start at any nucleotide in SEQ ID NO: 3 that allows for its full representation in SEQ ID NO: 3.

In certain embodiments, a functional fragment comprises any contiguous stretch of nucleotides in SEQ ID NO: 4 of at least 10, at least 11, at least 12, at least 13, at least 14, at least 15, at least 16, at least 17, at least 18, at least 19, at least 20, or at least 21 nucleotides in length. In certain embodiments, a functional fragment of SEQ ID NO: 4 comprises one, two, three, or four mismatches as compared to the corresponding contiguous stretch of nucleotides in SEQ ID NO: 4. A functional fragment may start at any nucleotide in SEQ ID NO: 4 that allows for its full representation in SEQ ID NO: 4.

In certain embodiments, a functional fragment comprises any contiguous stretch of nucleotides in SEQ ID NO: 5 of at least 10, at least 11, at least 12, at least 13, at least 14, at least 15, at least 16, at least 17, at least 18, at least 19, at least 20, or at least 21 nucleotides in length. In certain embodiments, a functional fragment of SEQ ID NO: 5 comprises one, two, three, or four mismatches as compared to the corresponding contiguous stretch of nucleotides in SEQ ID NO: 5. A functional fragment may start at any nucleotide in SEQ ID NO: 5 that allows for its full representation in SEQ ID NO: 5.

In certain embodiments, a functional fragment comprises any contiguous stretch of nucleotides in SEQ ID NO: 6 of at least 10, at least 11, at least 12, at least 13, at least 14, at least 15, at least 16, at least 17, at least 18, at least 19, at least 20, or at least 21 nucleotides in length. In certain embodiments, a functional fragment of SEQ ID NO: 6 comprises one, two, three, or four mismatches as compared to the corresponding contiguous stretch of nucleotides in SEQ ID NO: 6. A functional fragment may start at any nucleotide in SEQ ID NO: 6 that allows for its full representation in SEQ ID NO: 6.

In certain embodiments, a functional fragment comprises any contiguous stretch of nucleotides in SEQ ID NO: 7, SEQ ID NO: 8, SEQ ID NO: 9 or SEQ ID NO: 10 of at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100, at least 101, at least 102, at least 103, at least 104, at least 105, at least 106, at least 107, at least 108, at least 109, at least 110, at least 111, at least 112, at least 113, at least 114, at least 115, at least 116, at least 117, at least 118, at least 119, at least 120, at least 121, at least 122, at least 123, at least 124, at least 125, at least 126, at least 127, at least 128, at least 129, at least 130, at least 131, at least 132, at least 133, at least 134, at least 135, at least 136, at least 137, at least 138, at least 139, at least 140, at least 141, at least 142, at least 143, at least 144, at least 145, at least 146, at least 147, at least 148, at least 149, at least 150, at least 151, at least 152, at least 153, at least 154, at least 155, at least 156, at least 157, at least 158, at least 159, at least 160, at least 161, at least 162, at least 163, at least 164, at least 165, at least 166, at least 167, at least 168, at least 169, at least 170, at least 171, at least 172, at least 173, at least 174, at least 175, at least 176, at least 177, at least 178, at least 179, at least 180, at least 181, at least 182, at least 183, at least 184, at least 185, at least 186, at least 187, at least 188, at least 189, at least 190, at least 191, at least 192, at least 193, at least 194, at least 195, at least 196, at least 197, at least 198, at least 199, at least 200, at least 201, at least 202, at least 203, at least 204, at least 205, at least 206, at least 207, at least 208, at least 209, at least 210, at least 211, at least 212, at least 213, at least 214, at least 215, at least 216, at least 217, at least 218, at least 219, at least 220, at least 221, at least 222, at least 223, or at least 224 nucleotides in length. In certain embodiments, a functional fragment of SEQ ID NO: 7 or SEQ ID NO: 8 comprises one, two, three, four, five, six, seven, eight, nine, or ten mismatches as compared to the corresponding contiguous stretch of nucleotides in SEQ ID NO: 7, SEQ ID NO: 8, SEQ ID NO: 9 or SEQ ID NO: 10. A functional fragment may start at any nucleotide in SEQ ID NO: 7, SEQ ID NO: 8, SEQ ID NO: 9 or SEQ ID NO: 10.

In some embodiments, the RNA may comprise a miRNA binding site for a miRNA selected from hsa-mir-196b-5p, hsa-mir-10b-5p, hsa-mir-24-2-5p, hsa-mir-183-3p, hsa-mir-196a-5p, or hsa-mir-494-3p, or a complement thereof. In certain embodiments, the RNA may comprise one or more binding sites for a miRNA. In these embodiments, the RNA may comprise 6, 7, 8, 9 or 10 contiguous nucleotides which potentially base pair the seed region of a miRNA such as hsa-mir-196b-5p, hsa-mir-10b-5p, hsa-mir-24-2-5p, hsa-mir-183-3p, hsa-mir-196a-5p, or hsa-mir-494-3p (which is at the 5′ end of those miRNAs). In some embodiments, the RNA may comprise 6, 7, 8, 9 or 10 contiguous nucleotides at the 3′ end of any of SEQ ID NOS: 1-6, which potentially base pair the seed region of a miRNA such as hsa-mir-196b-5p, hsa-mir-10b-5p, hsa-mir-24-2-5p, hsa-mir-183-3p, hsa-mir-196a-5p, or hsa-mir-494-3p (which is at the 5′ end of those miRNAs). The one or more binding sites for a miRNA may comprise any of SEQ ID NOS: 1-6. In some embodiments, the sequence may be identical to SEQ ID NOS: 1-6 except that it has one, two, three of four mismatches relative to SEQ ID NOS: 1-6, for example.

Specifically, the sequence of: (i), (ii), or (iii) may provide a binding site for one or more of hsa-mir-196b-5p, hsa-mir-10b-5p, hsa-mir-24-2-5p, hsa-mir-183-3p, hsa-mir-196a-5p and hsa-mir-494-3p.

As would be apparent, the nucleic acid cassette itself (which is DNA) may contain (i) the DNA version of any of SEQ ID NOs. 1-10 and 43-48, (ii) a variant, a functional fragment, or a combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), wherein inclusion of the sequence reduces the expression of the protein or RNA encoded by the cassette in DRG cells in the organism, relative to a target tissue, e.g., a neural tissue, e.g., a neuronal cell in the brain.

In some embodiments, the sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), may result in decreased expression of the ncRNA or a polypeptide encoded by the mRNA in DRG cells as compared to expression of the ncRNA or the polypeptide encoded by the mRNA in DRG cells from an otherwise equivalent ncRNA or mRNA without the sequence of (i), (ii), or (iii). For example, an mRNA containing a sequence of (i), (ii), or (iii), may result in decreased expression of a polypeptide encoded by the mRNA in DRG cells at a level that is at least 1.5 fold, at least 2-fold, at least 5-fold, or at least 10-fold as compared to expression of the polypeptide in DRG cells from an otherwise equivalent mRNA without the sequence of (i), (ii), or (iii). In these embodiments, the reduction of expression of the polypeptide in DRG cells is greater than the reduction of expression of the polypeptide in the target cells when compared to otherwise equivalent mRNA without the sequence of (i), (ii), or (iii). Similarly, an ncRNA containing a sequence of (i), (ii), or (iii), may have decreased expression in DRG cells at a level that is at least 1.5 fold, at least 2-fold, at least 5-fold, or at least 10-fold as compared to expression of the ncRNA without the sequence of (i), (ii), or (iii) in DRG cells. In these embodiments, the reduction of expression of the ncRNA in DRG cells is greater than the reduction of expression of the ncRNA in the target cells when compared to otherwise equivalent ncRNA without the sequence of (i), (ii), or (iii).

In some embodiments, the sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), may result in decreased expression of a polypeptide encoded by the mRNA, the mRNA itself, or the ncRNA in DRG cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the polypeptide, mRNA, or ncRNA in DRG cells from an otherwise equivalent RNA transcript without the sequence of (i), (ii), or (iii). In these embodiments, the reduction of expression of the polypeptide or RNA transcript in DRG cells is greater than the reduction of expression of the polypeptide in the target cells when compared to otherwise equivalent RNA transcript without the sequence of (i), (ii), or (iii).

In some embodiments, the sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii), does not result in significantly decreased expression of a polypeptide encoded by the mRNA, the mRNA, or the ncRNA in target cells as compared to expression of the polypeptide, mRNA, or ncRNA in target cells from an otherwise equivalent RNA transcript without the sequence of (i), (ii), or (iii). In some embodiments, the sequence of (i), (ii), or (iii), may result in expression of a polypeptide encoded by the mRNA, the mRNA, or the ncRNA in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the polypeptide, mRNA, or ncRNA in target cells from an otherwise equivalent RNA transcript without the sequence of (i), (ii), or (iii). In these embodiments, the reduction of expression of the polypeptide in DRG cells is greater than the reduction of expression of the polypeptide, mRNA, or ncRNA in the target cells when compared to otherwise equivalent RNA transcript without the sequence of (i), (ii), or (iii).

In some embodiments, the RNA transcript is a therapeutic RNA transcript. In some aspects, the therapeutic RNA transcript is an mRNA that includes a sequence encoding a polypeptide, e.g., a therapeutic protein, which protein may be intracellular, membrane bound or secreted, for example. In some embodiments, the therapeutic protein one that is associated with a neural disease or disorder, e.g., a protein whose aberrant function (e.g., resulting from a genetic mutation or abnormality) is associated with a neural disease or disorder. In further embodiments, the therapeutic RNA transcript includes a ncRNA sequence that targets an endogenous molecule, e.g., a gene, protein, or RNA, associated with a neural disease or disorder.

Neural diseases and disorders include those associated with one or more genetic mutations as well as those with unknown etiologies. In some embodiments, neural diseases and disorders include conditions associated with epileptic seizures, neurodegenerative disorders, and/or neurodevelopmental disorders. Examples of neural diseases or disorders include, but are not limited to: Alpers-Huttenlocher Syndrome, Angelman Syndrome, CDKL5 Deficiency Disorder, Dravet Syndrome, Rett Syndrome, Parkinson's Disease and Parkinson's LIDS (side effect of Parkinson's medication), Alzheimer's disease, Creatine Transporter Deficiency, FOXG1 Syndrome, Fragile X Syndrome, Phelan-McDermid Syndrome, Childhood Absence Epilepsy, Childhood Epilepsy Centrotemporal Spikes (Benign Rolandic Epilepsy), Dravet Syndrome, Early Myoclonic Encephalopathy (EME), Epilepsy Eyelid Myoclonia Jeavons Syndrome, Epilepsy of Infancy with Migrating Focal Seizures, Epilepsy Myoclonic Absences, Epileptic Encephalopathy Continuous Spike and Wave During Sleep CSWS, Infantile Spasms (West Syndrome), Juvenile Myoclonic Epilepsy, Landau-Kleffner Syndrome, Lennox-Gastaut Syndrome (LGS), Myoclonic Epilepsy in Infancy, Ohtahara Syndrome, Panayiotopoulos Syndrome, Progressive Myoclonic Epilepsies, Reflex Epilepsies, Self-Limited Familial and Non-Familial Neonatal Infantile Seizures, Self-Limited Late Onset Occipital Epilepsy Gastaut Syndrome, Epilepsy Generalized Tonic Clonic Seizures Alone, Genetic Epilepsy with Febrile Seizures Plus, Juvenile Absence Epilepsy, Myoclonic Atonic Epilepsy Doose Syndrome, Sleep-related Hypermotor Epilepsy (SHE), febrile seizures, focal epilepsy, West Syndrome, Early Onset Epilepsy, Benign Familial Infantile Epilepsy, and Attention Deficit-Hyperactivity Disorder.

A number of genetic abnormalities have been associated with epilepsies, including many of the forgoing neural diseases and disorders. Examples of genes affected by these genetic abnormalities, i.e., genes whose activity and/or expression has been altered by genetic mutation(s), include: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX.

Thus, in embodiments in which the RNA transcript is an mRNA that includes a sequence that encodes a therapeutic protein for treatment of a neural disease or disorder, the therapeutic protein may be (i) a functional form of a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 80%, 81%, 82%, 83%, 84%, 85%, 86%, 87%, 88%, 89%, 90%, 91%, 92%, 93%, 94%, 95%, 96%, 97%, 98%, or 99% sequence identity to (i), (iii) a variant or functional fragment of (i) or (ii), or (iv) a transcription factor which modulates expression of a gene from (i). A transcription factor encoded by the mRNA may be an engineered transcription factor or a naturally occurring transcription factor.

In some cases, the target cells may be neural cells, muscle cells, cardiac cells, skin cells, immune cells, hematopoetic cells, cancer cells, pancreatic cells, or kidney cells. In any of these embodiments, the target cells may be neural cells, e.g., cerebrum cells, brainstem cells, hippocampus cells, or cerebellum cells. For example, in these embodiments, the neural cells may be GABAergic cells, e.g., parvalbumin expressing cells. In some cases, the target cell may be a CNS cell, such as an excitatory neuron, a dopaminergic neuron, a glial cell, an ependymal cell, an oligodendrocyte, an astrocyte, a microglia, a motor neuron, a vascular cell, a GABAergic neuron, or a non-GABAergic neuron (e.g., a cell that does not express one or more of GAD2, GAD1, NKX2.1, DLX1, DLX5, SST and VIP), a non-PV neuron (e.g., a GABAergic neuron that does not express parvalbumin), or another CNS cell (e.g., a CNS cell type that has never expressed any of PV, GAD2, GAD1, NKX2.1, DLX1, DLX5, SST, and VIP).

The cassette may be linear, circular and, in some embodiments, the nucleic acid cassette may be a vector such as a plasmid or viral vector, e.g., an adeno-associated virus (AAV) vector or lentiviral vector. In particular embodiments, the viral vector may be an AAV vector selected from AAV1, AAV2, AAV3, AAV4, AAV5, AAV6, AAV7, AAV8, AAV9, AAV9.47, AAV9 (hul4), AAV10, AAV11, AAV 12, AAV13, AAVrh8, AAVrhlO, AAV-DJ, and AAV-DJ8, and hybrids thereof.

Also provided is a nucleic acid cassette comprising a transgene encoding an RNA transcript, wherein the RNA comprises a miRNA binding site for a miRNA selected from mir-196b-5p, mir-10b-5p, mir-24-2-5p, mir-183-3p, mir-196a-5p, mir-494-3p, or a complement thereof. In some embodiments, the RNA may comprise a binding site for a miRNA produced from mir-196b, mir-10b, mir-24-2, mir-183, mir-196a, or mir-494 gene. The miRNA binding site should not be in the naturally occurring version of the RNA, if the RNA is otherwise naturally occurring. In some embodiments, the cassette may comprise two or more, three or more or four or more binding sites for miRNAs selected from mir-196b-5p, mir-10b-5p, mir-24-2-5p, and mir-183-3p, mir-196a-5p, mir-494-3p, or a complement thereof, for example. In some embodiments, the RNA is an mRNA, e.g., an mRNA encoding a therapeutic protein (as described elsewhere herein). In these embodiments, the binding sites may be anywhere in the mRNA, particularly in a non-coding sequence such as a 3′ UTR region, a 5′ UTR, an intron, or any combination thereof.

In any embodiment, the nucleic acid cassette may be non-naturally occurring, meaning that, for example, the miRNA binding site in the RNA transcript expressed from the nucleic acid cassette is heterologous to one or more other regions of the RNA transcript. In any embodiment, the nucleic acid cassette may comprise a promoter and/or enhancer. In some embodiments, this nucleic acid cassette may be composed of a promoter, a coding sequence, and a terminator, where the promoter, coding sequence and terminator are in operable linkage. In these embodiments, the promoter may be heterologous to the coding sequence, meaning that the promoter does not drive the expression of that coding sequence in a wild type cell. In any embodiment, the nucleic acid cassette may additionally comprise an enhancer.

In some embodiments, the mRNA may encode a polypeptide, e.g., a therapeutic protein, which protein may be intracellular, membrane bound or secreted, for example.

In some embodiments, the polypeptide is a therapeutic protein whose altered function (e.g., by a genetic mutation) is associated with a neural disease or disorder. As noted above, neural diseases and disorders include those associated with one or more genetic mutations as well as those with unknown etiologies. Examples of neural diseases and disorders include conditions associated with epileptic seizures, neurodegenerative disorders, and/or neurodevelopmental disorders. Examples of neural diseases or disorders include, but are not limited to: Alpers-Huttenlocher Syndrome, Angelman Syndrome, CDKL5 Deficiency Disorder, Dravet Syndrome, Rett Syndrome, Parkinson's Disease and Parkinson's LIDS (side effect of Parkinson's medication), Alzheimer's disease, Creatine Transporter Deficiency, FOXG1 Syndrome, Fragile X Syndrome, Phelan-McDermid Syndrome, Childhood Absence Epilepsy, Childhood Epilepsy Centrotemporal Spikes (Benign Rolandic Epilepsy), Dravet Syndrome, Early Myoclonic Encephalopathy (EME), Epilepsy Eyelid Myoclonia Jeavons Syndrome, Epilepsy of Infancy with Migrating Focal Seizures, Epilepsy Myoclonic Absences, Epileptic Encephalopathy Continuous Spike and Wave During Sleep CSWS, Infantile Spasms (West Syndrome), Juvenile Myoclonic Epilepsy, Landau-Kleffner Syndrome, Lennox-Gastaut Syndrome (LGS), Myoclonic Epilepsy in Infancy, Ohtahara Syndrome, Panayiotopoulos Syndrome, Progressive Myoclonic Epilepsies, Reflex Epilepsies, Self-Limited Familial and Non-Familial Neonatal Infantile Seizures, Self-Limited Late Onset Occipital Epilepsy Gastaut Syndrome, Epilepsy Generalized Tonic Clonic Seizures Alone, Genetic Epilepsy with Febrile Seizures Plus, Juvenile Absence Epilepsy, Myoclonic Atonic Epilepsy Doose Syndrome, Sleep-related Hypermotor Epilepsy (SHE), febrile seizures, focal epilepsy, West Syndrome, Early Onset Epilepsy, Benign Familial Infantile Epilepsy, and Attention Deficit-Hyperactivity Disorder.

In these embodiments, the therapeutic protein may be (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 80%, 81%, 82%, 83%, 84%, 85%, 86%, 87%, 88%, 89%, 90%, 91%, 92%, 93%, 94%, 95%, 96%, 97%, 98%, or 99% sequence identity to (i), (iii) a functional fragment of (i) or (ii), or (iv) a transcription factor which modulates expression of a gene from (i). A transcription factor encoded by the mRNA may be an engineered transcription factor or a naturally occurring transcription factor.

In some cases, the target cells may be neural cells, muscle cells, cardiac cells, skin cells, immune cells, hematopoctic cells, cancer cells, pancreatic cells, or kidney cells. In any of these embodiments, the target cells may be neural cells, e.g., cerebrum cells, brainstem cells, hippocampus cells, or cerebellum cells. For example, in these embodiments, the neural cells may be GABAergic cells, e.g., parvalbumin expressing cells. In some cases, the target cell may be a CNS cell, such as an excitatory neuron, a dopaminergic neuron, a glial cell, an ependymal cell, an oligodendrocyte, an astrocyte, a microglia, a motor neuron, a vascular cell, a GABAergic neuron, or a non-GABAergic neuron (e.g., a cell that does not express one or more of GAD2, GAD1, NKX2.1, DLX1, DLX5, SST and VIP), a non-PV neuron (e.g., a GABAergic neuron that does not express parvalbumin), or another CNS cell (e.g., a CNS cell type that has never expressed any of PV, GAD2, GAD1, NKX2.1, DLX1, DLX5, SST, and VIP).

The cassette may be linear, circular and, in some embodiments, the nucleic acid cassette may be a vector such as a plasmid or viral vector, e.g., an adeno-associated virus (AAV) vector or lentiviral vector. In particular embodiments, the viral vector may be AAV vector selected from is AAV1, AAV2, AAV3, AAV4, AAV5, AAV6, AAV7, AAV8, AAV9, AAV9.47, AAV9 (hul4), AAV10, AAV11, AAV 12, AAV13, AAVrh8, AAVrh10, AAV-DJ, and AAV-DJ8, and hybrids thereof.

Also provided is an RNA transcript encoded by a nucleic acid cassette described herein.

Also provided is a method of decreasing DRG expression of a polypeptide encoded by an mRNA, an mRNA, or an ncRNA relative to expression of the polypeptide, mRNA, or ncRNA in a target tissue. In these embodiments, the method may comprise constructing a nucleic acid cassette to include a DRG de-targeting sequence as described herein in an RNA transcript encoded therein. For example, the nucleic acid cassette can be constructed to include a sequence of (i) one of SEQ ID NOs 1-10 and 43-48, (ii) a variant, functional fragment, or a combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii) in the RNA transcript encoded therein. Details of the cassettes made by this method are described herein. In some embodiments, the method may comprise introducing an expression cassette as described herein, or an RNA encoded thereby, into an organism, e.g., a human subject, wherein inclusion of any one or more of the disclosed DRG de-targeting sequences reduces the expression of the protein in DRG cells in the organism, relative to a target tissue.

In any embodiment herein, the target cells may be neural cells, muscle cells, cardiac cells, skin cells, immune cells, hematopoetic cells, cancer cells, pancreatic cells, or kidney cells. In some cases, the target cells may be neural cells, e.g., cerebrum cells, brainstem cells, hippocampus cells, or cerebellum cells. For example, in some embodiments, the neural cells are GABAergic cells, e.g., parvalbumin expressing cells. In some cases, the target cell may be a CNS cell, such as an excitatory neuron, a dopaminergic neuron, a glial cell, an ependymal cell, an oligodendrocyte, an astrocyte, a microglia, a motor neuron, a vascular cell, a GABAergic neuron, or a non-GABAergic neuron (e.g., a cell that does not express one or more of GAD2, GAD1, NKX2.1, DLX1, DLX5, SST and VIP), a non-PV neuron (e.g., a GABAergic neuron that does not express parvalbumin), or other CNS cells (e.g., CNS cell types that have never expressed any of PV, GAD2, GAD1, NKX2.1, DLX1, DLX5, SST and VIP).

In any embodiment, the method may further comprise administering a vector (e.g., an AAV or lentiviral vector) encoding the RNA transcript to a subject, e.g., wherein the RNA transcript is an mRNA that encodes a therapeutic protein. In some embodiments, the method may comprise administering the RNA transcript to a subject.

### Expression Cassettes

A nucleic acid cassette may contain one or more additional regulatory elements (e.g., a promoter, a terminator, and/or an enhancer, etc.) that induces expression of transgene in a particular cell type, or a particular class of cell types. For instance, a cell type selective regulatory element can induce gene expression in a particular cell type relative to one or more other cell types. Alternatively or in addition, a cell type selective regulatory element can induce gene expression in a particular class of cells relative to one or more other classes of cells. In one embodiment, a cell type selective regulatory element of the invention enhances gene expression in a particular cell type, or a particular class of cells. In another embodiment, a cell type selective regulatory element suppresses gene expression in a particular cell type, or a particular class of cells. Cell type selective modulation of gene expression (e.g., enhancing or suppressing gene expression) does not require that gene expression is affected only in the target cell type or class of cells. Rather, cell type selective modulation of gene expression (e.g., enhancing or suppressing gene expression) requires only that gene expression increase, or decrease, in the target cell type relative to one or more other cell types, or classes of cells.

In one embodiment, the application provides an expression cassette comprising a promoter operably linked to nucleic acid sequence encoding an RNA transcript, wherein the RNA transcript comprises DRG de-targeting region comprising (i) SEQ ID NO: 1; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 2; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 3; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 4; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 5; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 6; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 7; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 8; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 9; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 10; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) at least two different sequences selected from SEQ ID NOs. 1-10 and 43-48; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) at least three different sequences selected from SEQ ID NOs. 1-10 and 43-48; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) at least four different sequences selected from SEQ ID NOs. 1-10 and 43-48; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) at least five different sequences selected from SEQ ID NOs. 1-10 and 43-48; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NOs. 1-10 and 43-48, in any order; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 43; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 44; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 45; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 46; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 47; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises DRG de-targeting region comprising (i) SEQ ID NO: 48; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In some embodiments, the nucleic acid cassette may comprise a CNS selective promoter that is operably linked to a polynucleotide encoding a therapeutic protein and one or more DRG de-targeting element/sequence as disclosed herein. CNS promoters are promoters that specifically modulate gene expression in one or more cells of the central nervous system. For example, CNS selective promoters may specifically modulate gene expression in one or more neurons or glial cells of the CNS. In one embodiment, CNS selective promoters specifically modulate gene expression in one or more neurons or astrocytes. In another embodiment, CNS selective promoters specifically modulate gene expression in one or more astrocytes. In certain embodiments, CNS selective promoters enhance expression in a CNS cell (e.g., a neuron, or a glial cell such as an astrocyte) relative to one or more other CNS cell types (e.g., excitatory neurons, dopaminergic neurons, microglia, motor neurons, vascular cells, non-GABAergic neurons, or other CNS cells).

Examples of CNS selective promoters include, but are not limited to: Ca2+/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters. Suitable promoters are also described in, e.g., WO 2018/187363, which sequences are incorporated by reference herein. Other sequences may be used.

In some embodiments, the cassette may comprise a GABAergic neuron selective promoter that is operably linked to a polynucleotide encoding a therapeutic protein. GABAergic cells are inhibitory neurons which produce gamma-aminobutyric acid. GABAergic cells can be identified by markers such as the expression of glutamic acid decarboxylase 2 (GAD2), GAD1, NKX2.1, DLX1, DLX5, SST, PV, and VIP. GABAergic neuron selective promoters are regulatory elements that specifically modulate gene expression in a GABAergic neuron. For example, GABAergic neuron-selective promoter enhance expression in a GABAergic neuron relative to one or more other CNS cell types (e.g., excitatory neurons, dopaminergic neurons, astrocytes, microglia, motor neurons, vascular cells, non-GABAergic neurons, or other CNS cells).

PV neuron selective promoters are promoters that specifically modulate gene expression in a PV neuron. For example, PV neuron selective promoters enhance expression in a PV neuron relative to one or more other CNS cell types.

In certain embodiments, a neuron selective promoter may be human derived or comprises a sequence that is human derived. In some cases, the promoter may be mouse derived or comprises a sequence that is mouse derived. In some cases, the promoter is non-naturally occurring or comprises a non-naturally occurring sequence. In some instances, the sequence of a promoter may be 100% human derived. In other instances, at least 5%, 10%, 20%, 30%, 40%, 50%, 60%, 70%, 80%, 90%, 95%, 98%, or 99% of the promoter sequence is human derived. For example, a promoter can have 50% of its sequence derived from human, and the remaining 50% be non-human derived (e.g., mouse derived or fully synthetic).

In some embodiments, the therapeutic protein encoded by the mRNA is associated with a neural disease or disorder. As noted above, neural diseases and disorders include those associated with one or more genetic mutations as well as those with unknown etiologies. Examples of neural diseases and disorders include conditions associated with epileptic seizures, neurodegenerative disorders, and/or neurodevelopmental disorders. Examples of neural diseases or disorders include, but are not limited to: Alpers-Huttenlocher Syndrome, Angelman Syndrome, CDKL5 Deficiency Disorder, Dravet Syndrome, Rett Syndrome, Parkinson's Disease and Parkinson's LIDS (side effect of Parkinson's medication), Alzheimer's disease, Creatine Transporter Deficiency, FOXG1 Syndrome, Fragile X Syndrome, Phelan-McDermid Syndrome, Childhood Absence Epilepsy, Childhood Epilepsy Centrotemporal Spikes (Benign Rolandic Epilepsy), Dravet Syndrome, Early Myoclonic Encephalopathy (EME), Epilepsy Eyelid Myoclonia Jeavons Syndrome, Epilepsy of Infancy with Migrating Focal Seizures, Epilepsy Myoclonic Absences, Epileptic Encephalopathy Continuous Spike and Wave During Sleep CSWS, Infantile Spasms (West Syndrome), Juvenile Myoclonic Epilepsy, Landau-Kleffner Syndrome, Lennox-Gastaut Syndrome (LGS), Myoclonic Epilepsy in Infancy, Ohtahara Syndrome, Panayiotopoulos Syndrome, Progressive Myoclonic Epilepsies, Reflex Epilepsies, Self-Limited Familial and Non-Familial Neonatal Infantile Seizures, Self-Limited Late Onset Occipital Epilepsy Gastaut Syndrome, Epilepsy Generalized Tonic Clonic Seizures Alone, Genetic Epilepsy with Febrile Seizures Plus, Juvenile Absence Epilepsy, Myoclonic Atonic Epilepsy Doose Syndrome, Sleep-related Hypermotor Epilepsy (SHE), febrile seizures, focal epilepsy, West Syndrome, Early Onset Epilepsy, Benign Familial Infantile Epilepsy, and Attention Deficit-Hyperactivity Disorder.

In these embodiments, the therapeutic protein may be (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 80%, 81%, 82%, 83%, 84%, 85%, 86%, 87%, 88%, 89%, 90%, 91%, 92%, 93%, 94%, 95%, 96%, 97%, 98%, or 99% sequence identity to (i), (iii) a functional fragment of (i) or (ii), or (iv) a transcription factor which modulates expression of a gene from (i). A transcription factor encoded by the mRNA may be an engineered transcription factor or a naturally occurring transcription factor that modulates, e.g., activates or represses, a gene of interest.

In certain embodiments, the nucleic acid constructs described herein comprise another regulatory element in an addition to a promoter, such as, for example, sequences associated with transcription initiation or termination, enhancer sequences, and efficient RNA processing signals. Exemplary regulatory elements include, for example, an intron, an enhancer, UTR, stability element, WPRE sequence, a Kozak consensus sequence, posttranslational response element, or a polyadenylation (polyA) sequence, or a combination thereof. Regulatory elements can function to modulate gene expression at the transcriptional phase, post-transcriptional phase, or at the translational phase of gene expression. At the RNA level, regulation can occur at the level of translation (e.g., stability elements that stabilize mRNA for translation), RNA cleavage, RNA splicing, and/or transcriptional termination. In various embodiments, regulatory elements can recruit transcription factors to a coding region that increase gene expression selectivity in a cell type of interest, increase the rate at which RNA transcripts are produced, increase the stability of RNA produced, and/or increase the rate of protein synthesis from RNA transcripts.

In certain embodiments, the cassette may further comprise a polyA sequence. Suitable polyA sequences include, for example, an artificial polyA that is about 75 bp in length (PA75) (see e.g., WO 2018/126116), the bovine growth hormone polyA, SV40 early polyA signal, SV40 late polyA signal, rabbit beta globin polyA, HSV thymidine kinase polyA, protamine gene polyA, adenovirus 5 Elb polyA, growth hormone polyA, or a PBGD polyA. In certain embodiments, the polyA sequence is positioned downstream of the polynucleotide encoding a functional therapeutic protein in the nucleic acid constructs described herein.

### Liver De-Targeting

In addition to the DRG de-targeting embodiments above, the present disclosure further includes liver de-targeting elements. The liver de-targeting elements provided can be present in nucleic acid cassettes, RNA molecules, e.g., RNA transcripts, synthetic RNA molecules, and the like, as described above for DRG de-targeting elements. In addition, methods for the use of the liver de-targeting elements to reduce the expression and/or activity of a transgene in liver cells/tissues are provided. Such methods are similar to those detailed above for methods for the use of the DRG de-targeting elements to reduce the expression and/or activity of a transgene in DRG cells. Thus, all embodiments described herein for DRG de-targeting elements can be applied to the liver de-targeting elements described below, with the understanding that the tissue/cells being de-targeted by the liver de-targeting elements is live tissue/liver cells and not DRG cells.

Thus, in certain embodiments, a nucleic acid cassette comprising a therapeutic transgene encoding an RNA transcript, wherein the RNA transcript comprises a sequence of (i) any SEQ ID NOS. 65, 110, and 112, (ii) a variant, functional fragment, or combination thereof, or (iii) a sequence at least 80% identical to (i) or (ii) is provided. In these embodiments, the sequence decreases expression of the RNA transcript in liver cells. Embodiments that make use of these sequences are described in greater detail below. These liver de-targeting sequences can be employed in any embodiment in which de-targeting in liver is desirable.

In one embodiment, the application provides an expression cassette comprising a promoter operably linked to nucleic acid sequence encoding an RNA transcript, wherein the RNA transcript comprises liver de-targeting region comprising (i) SEQ ID NO: 65; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises liver de-targeting region comprising (i) SEQ ID NO: 110; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises liver de-targeting region comprising (i) SEQ ID NO:112; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises liver de-targeting region comprising (i) at least two different sequences selected from SEQ ID NOS. 65, 110, and 112; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises liver de-targeting region comprising (i) at least three different sequences selected from SEQ ID NOS. 65, 110, and 112; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

In one embodiment, the application provides an expression cassette comprising a promoter operably linked to nucleic acid sequence encoding an RNA transcript, wherein the RNA transcript comprises liver de-targeting region comprising (i) at least four different sequences selected from SEQ ID NOS. 65, 110, and 112; (ii) a variant, functional fragment, multiple copies, or a combination thereof; or (iii) a nucleic acid sequence having at least 80%, 81%, 82%, 83%, 84%, 85%, 86%, 87%, 88%, 89%, 90%, 91%, 92%, 93%, 94%, 95%, 96%, 97%, 98%, or 99% sequence identity to any one of (i) or (ii). In certain embodiments, the promoter is a tissue selective or tissue specific promoter. In certain embodiments, the promoter is a CNS selective promoter and the RNA transcript is a therapeutic expression product for a neural disease or disorder, e.g., an mRNA that encodes a therapeutic protein.

### Sequence Combinations

As illustrated in any of the following tables, the DRG and liver targeting sequences described above may be combined with one another or with other de-targeting sequences to produce a cassette that more effectively de-targets a single tissue (i.e., DRG or liver) or a combination of tissues (i.e., DRG and liver). In the following tables, an “X” indicates a combination of a first sequence in the x axis and a second sequence in the y axis where, in any combination, the combination may contain a single copy of the first sequence, two copies of the first sequence, three copies of the first sequence, four copies of the first sequence or at least five copies of the first sequence and, independently, a single copy of the second sequence, two copies of the second sequence, three copies of the second sequence, four copies of the second sequence or at least five copies of the second sequence.

Table 1 below shows exemplary combinations of the DRG de-targeting elements (SEQ ID NOS. 1-10 and 43-48) that could be employed herein.

TABLE 1

| SEQ ID | 1 | 2 | 3 | 4 | 5 | 6 | 7 | 8 | 9 | 10 | 43 | 44 | 45 | 46 | 47 | 48 |
|---|---|---|---|---|---|---|---|---|---|---|---|---|---|---|---|---|
| 1 | | X | X | X | X | X | X | X | X | X | X | X | X | X | X | X |
| 2 | X | | X | X | X | X | X | X | X | X | X | X | X | X | X | X |
| 3 | X | X | | X | X | X | X | X | X | X | X | X | X | X | X | X |
| 4 | X | X | X | | X | X | X | X | X | X | X | X | X | X | X | X |
| 5 | X | X | X | X | | X | X | X | X | X | X | X | X | X | X | X |
| 6 | X | X | X | X | X | | X | X | X | X | X | X | X | X | X | X |
| 7 | X | X | X | X | X | X | | X | X | X | X | X | X | X | X | X |
| 8 | X | X | X | X | X | X | X | | X | X | X | X | X | X | X | X |
| 9 | X | X | X | X | X | X | X | X | | X | X | X | X | X | X | X |
| 10 | X | X | X | X | X | X | X | X | X | | X | X | X | X | X | X |
| 43 | X | X | X | X | X | X | X | X | X | X | | X | X | X | X | X |
| 44 | X | X | X | X | X | X | X | X | X | X | X | | X | X | X | X |
| 45 | X | X | X | X | X | X | X | X | X | X | X | X | | X | X | X |
| 46 | X | X | X | X | X | X | X | X | X | X | X | X | X | | X | X |
| 47 | X | X | X | X | X | X | X | X | X | X | X | X | X | X | | X |
| 48 | X | X | X | X | X | X | X | X | X | X | X | X | X | X | X | |

The liver de-targeting elements of SEQ ID NOS. 65, 110, and 112 can be combined with one another or with the liver de-targeting elements set forth in PCT/US2023065801, filed on Apr. 14, 2023 (i.e., SEQ ID NOs. 57-62, 64, and 66-71 in this application), and incorporated by reference herein.

Table 2 below shows exemplary combinations of the liver de-targeting elements that could be employed herein.

TABLE 2

| SEQ ID | 57 | 58 | 59 | 60 | 61 | 62 | 64 | 65 | 66 | 67 | 68 | 69 | 70 | 71 | 110 | 112 |
|---|---|---|---|---|---|---|---|---|---|---|---|---|---|---|---|---|
| 57 | | X | X | X | X | X | X | X | X | X | X | X | X | X | X | X |
| 58 | X | | X | X | X | X | X | X | X | X | X | X | X | X | X | X |
| 59 | X | X | | X | X | X | X | X | X | X | X | X | X | X | X | X |
| 60 | X | X | X | | X | X | X | X | X | X | X | X | X | X | X | X |
| 61 | X | X | X | X | | X | X | X | X | X | X | X | X | X | X | X |
| 62 | X | X | X | X | X | | X | X | X | X | X | X | X | X | X | X |
| 64 | X | X | X | X | X | X | | X | X | X | X | X | X | X | X | X |
| 65 | X | X | X | X | X | X | X | | X | X | X | X | X | X | X | X |
| 66 | X | X | X | X | X | X | X | X | | X | X | X | X | X | X | X |
| 67 | X | X | X | X | X | X | X | X | X | | X | X | X | X | X | X |
| 68 | X | X | X | X | X | X | X | X | X | X | | X | X | X | X | X |
| 69 | X | X | X | X | X | X | X | X | X | X | X | | X | X | X | X |
| 70 | X | X | X | X | X | X | X | X | X | X | X | X | | X | X | X |
| 71 | X | X | X | X | X | X | X | X | X | X | X | X | X | | X | X |
| 110 | X | X | X | X | X | X | X | X | X | X | X | X | X | X | | X |
| 112 | X | X | X | X | X | X | X | X | X | X | X | X | X | X | X | |

In any embodiment, the nucleic acid cassette may comprise a therapeutic transgene encoding an RNA transcript (e.g., an mRNA), wherein the RNA transcript includes a first sequence that de-targets expression in DRG cells and a second sequence that de-targets expression in liver cells. In these embodiments, the first and second sequences may result in decreased expression of the RNA transcript or a polypeptide encoded by the same (e.g., when the RNA transcript is an mRNA) in DRG and liver cells relative to a target tissue, e.g., neural cells such as cerebrum cells, brainstem cells, hippocampus cells, cerebellum cells, or GABAergic cells, e.g., GABAergic cells are parvalbumin expressing cells.

In this cassette, wherein the first and second sequences may result in decreased expression of the RNA transcript or a polypeptide encoded by the same (e.g., when the RNA transcript is an mRNA) in DRG cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript or polypeptide in DRG cells from an otherwise equivalent RNA transcript without the first and second sequences and, independently, decreased expression of the RNA transcript or a polypeptide encoded by the same in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript or polypeptide in liver cells from an otherwise equivalent RNA transcript without the first and second sequences.

In any of these embodiments, (a) the first sequence may be: (i) any of SEQ ID NOS. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii); and (b) the second sequence is: (iv) any of SEQ ID NOs. 57-62, 64-71, 110, and 112, (v) a variant, functional fragment, or combination thereof, or (vi) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (iv) or (v).

In some of these embodiments, the first sequence of: (i), (ii), or (iii) may provide a binding site for one or more of hsa-mir-196b-5p, hsa-mir-10b-5p, hsa-mir-24-2-5p, hsa-mir-183-3p, hsa-mir-196a-5p and hsa-mir-494-3p and the second sequence of: (iv), (v), or (vi) may provide a binding site for hsa-mir-22-3p, hsa-mir-1258, hsa-mir-5589-3p, hsa-mir-17-5p, hsa-mir-203a-3p, hsa-mir-122-3p, hsa-mir-93-5p, and hsa-mir-19a-3p.

In any of these embodiments, the RNA transcript may comprise a combination of sequences selected from of Table 3 below where, in Table 3, SEQ ID NOS: 1-10 and 43-48 de-target expression in DRG cells, whereas SEQ ID NOS 57-62, 64-71, 110, and 112 de-target expression in liver cells. Table 3 below shows exemplary combinations of DRG de-targeting and liver de-targeting sequence elements that could be employed herein.

TABLE 3

| SEQ ID | 1 | 2 | 3 | 4 | 5 | 6 | 7 | 8 | 9 | 10 | 43 | 44 | 45 | 46 | 47 | 48 |
|---|---|---|---|---|---|---|---|---|---|---|---|---|---|---|---|---|
| 57 | X | X | X | X | X | X | X | X | X | X | X | X | X | X | X | X |
| 58 | X | X | X | X | X | X | X | X | X | X | X | X | X | X | X | X |
| 59 | X | X | X | X | X | X | X | X | X | X | X | X | X | X | X | X |
| 60 | X | X | X | X | X | X | X | X | X | X | X | X | X | X | X | X |
| 61 | X | X | X | X | X | X | X | X | X | X | X | X | X | X | X | X |
| 62 | X | X | X | X | X | X | X | X | X | X | X | X | X | X | X | X |
| 64 | X | X | X | X | X | X | X | X | X | X | X | X | X | X | X | X |
| 65 | X | X | X | X | X | X | X | X | X | X | X | X | X | X | X | X |
| 66 | X | X | X | X | X | X | X | X | X | X | X | X | X | X | X | X |
| 67 | X | X | X | X | X | X | X | X | X | X | X | X | X | X | X | X |
| 68 | X | X | X | X | X | X | X | X | X | X | X | X | X | X | X | X |
| 69 | X | X | X | X | X | X | X | X | X | X | X | X | X | X | X | X |
| 70 | X | X | X | X | X | X | X | X | X | X | X | X | X | X | X | X |
| 71 | X | X | X | X | X | X | X | X | X | X | X | X | X | X | X | X |
| 110 | X | X | X | X | X | X | X | X | X | X | X | X | X | X | X | X |
| 112 | X | X | X | X | X | X | X | X | X | X | X | X | X | X | X | X |

For clarity, an RNA transcript can comprise multiple different DRG de-targeting elements combined with one or more liver de-targeting elements and/or multiple different liver de-targeting elements combined with one or more DRG de-targeting elements, each element of which may be independently present in the RNA transcript in one or multiple (e.g., two, three, four or five or more) copies. SEQ ID NO: 111 is an example of such a combination although there are several others (as illustrated in the experimental section of this disclosure).

The first and second sequences reduce the expression of the transgene in dorsal root ganglion cells (DRG) and liver cells relative to target cells (such as neural cells, e.g., neurons) and as such, may be employed in a variety of gene therapy strategics that target cells that are not in the DRG or liver. Reducing expression of the transgene in DRG and liver cells relative to target cells means that the reduction in transgene expression driven by the DRG and liver de-targeting sequences disclosed herein is greater in DRG cells and liver than in the target cells. As such, while reduced transgene expression in target cells may be observed in certain embodiments, it is less than that observed in DRG and liver cells. This reduction in expression in the DRG and liver can reduce or eliminate toxicity and/or axonopathy in a subject receiving a gene therapy targeted to a non-DRG cell or tissue, e.g., neural cells, e.g., neurons, and non-liver cells, thereby improving its safety profile.

In some embodiments, the DRG and/or liver de-targeting elements disclosed herein can be used in combination with other sequences that have known cell- or tissue-specific de-targeting activity. For example, an expression cassette of the present disclosure can encode an RNA transcript that includes one or more de-targeting sequences disclosed herein (e.g., SEQ ID NOs: 1-10, 57-62, and 64-71, either alone or in any combination) and also include one or more sequences having known de-targeting activity, e.g., SEQ ID NO: 63, which has liver de-targeting activity. No limitation in this regard is intended.

### Vectors

Expression vectors may be used to deliver the nucleic acid molecule to a target cell via transfection or transduction. A vector may be an integrating or non-integrating vector, referring to the ability of the vector to integrate the expression cassette or transgene into the genome of the host cell. Examples of expression vectors include, but are not limited to, (a) non-viral vectors such as nucleic acid vectors including linear oligonucleotides and circular plasmids; artificial chromosomes such as human artificial chromosomes (HACs), yeast artificial chromosomes (YACs), and bacterial artificial chromosomes (BACs or PACs)); episomal vectors; transposons (e.g., PiggyBac); and (b) viral vectors such as retroviral vectors, lentiviral vectors, adenoviral vectors, and adeno-associated viral vectors.

Expression vectors may be linear oligonucleotides or circular plasmids and can be delivered to a cell via various transfection methods, including physical and chemical methods. Physical methods generally refer to methods of delivery employing a physical force to counteract the cell membrane barrier in facilitating intracellular delivery of genetic material. Examples of physical methods include the use of a needle, ballistic DNA, electroporation, sonoporation, photoporation, magnetofection, and hydroporation. Chemical methods generally refer to methods in which chemical carriers deliver a nucleic acid molecule to a cell and may include inorganic particles, lipid-based vectors, polymer-based vectors, and peptide-based vectors.

In some embodiments, an expression vector is administered to a target cell using an inorganic particle. Inorganic particles may refer to nanoparticles, such as nanoparticles that are engineered for various sizes, shapes, and/or porosity to escape from the reticuloendothelial system or to protect an entrapped molecule from degradation. Inorganic nanoparticles can be prepared from metals (e.g., iron, gold, and silver), inorganic salts, or ceramics (e.g., phosphate or carbonate salts of calcium, magnesium, or silicon). The surface of these nanoparticles can be coated to facilitate DNA binding or targeted gene delivery. Magnetic nanoparticles (e.g., supermagnetic iron oxide), fullerenes (e.g., soluble carbon molecules), carbon nanotubes (e.g., cylindrical fullerenes), quantum dots and supramolecular systems may also be used.

In some embodiments, an expression vector is administered to a target cell using a cationic lipid (e.g., cationic liposome). Various types of lipids have been investigated for gene delivery, such as, for example, a lipid nano emulsion (e.g., which is a dispersion of one immiscible liquid in another stabilized by emulsifying agent), or a solid lipid nanoparticle.

In some embodiments, an expression vector is administered to a target cell using a peptide-based delivery vehicle. Peptide based delivery vehicles can have advantages of protecting the genetic material to be delivered, targeting specific cell receptors, disrupting endosomal membranes, and delivering genetic material into a nucleus. In some embodiments, an expression vector is administered to a target cell using a polymer-based delivery vehicle. Polymer based delivery vehicles may comprise natural proteins, peptides, and/or polysaccharides or synthetic polymers. In one embodiment, a polymer-based delivery vehicle comprises polyethylenimine (PEI). PEI can condense DNA into positively charged particles which bind to anionic cell surface residues and are brought into the cell via endocytosis. In other embodiments, a polymer based delivery vehicle may comprise poly-L-lysine (PLL), poly (DL-lactic acid) (PLA), poly (DL-lactide-co-glycoside) (PLGA), polyornithine, polyarginine, histones, protamines, dendrimers, chitosans, synthetic amino derivatives of dextran, and/or cationic acrylic polymers. In certain embodiments, polymer-based delivery vehicles may comprise a mixture of polymers, such as, for example PEG and PLL.

In certain embodiments, an expression vector may be a viral vector suitable for gene therapy. Preferred characteristics of viral gene therapy vectors or gene delivery vectors may include the ability to be reproducibly and stably propagated and purified to high titres; to mediate targeted delivery (e.g., to deliver the transgene specifically to the tissue or organ of interest without widespread vector dissemination elsewhere); and to mediate gene delivery and transgene expression without inducing harmful side effects.

Several types of viruses, for example the non-pathogenic parvovirus referred to as adeno-associated virus, have been engineered for the purposes of gene therapy by harnessing the viral infection pathway but avoiding the subsequent expression of viral genes that can lead to replication and toxicity. Such viral vectors can be obtained by deleting all, or some, of the coding regions from the viral genome, but leaving intact those sequences (e.g., terminal repeat sequences) that may be necessary for functions such as packaging the vector genome into the virus capsid or the integration of vector nucleic acid (e.g., DNA) into the host chromatin.

In various embodiments, suitable viral vectors include retroviruses (e.g., A-type, B-type, C-type, and D-type viruses), adenovirus, parvovirus (e.g. adeno-associated viruses or AAV), coronavirus, negative strand RNA viruses such as orthomyxovirus (e.g., influenza virus), rhabdovirus (e.g., rabies and vesicular stomatitis virus), paramyxovirus (e. g. measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Examples of retroviruses include avian leukosis-sarcoma virus, human T-lymphotrophic virus type 1 (HTLV-1), bovine leukemia virus (BLV), lentivirus, and spumavirus. Other viruses include Norwalk virus, togavirus, flavivirus, reoviruses, papovavirus, hepadnavirus, and hepatitis virus, for example. Viral vectors may be classified into two groups according to their ability to integrate into the host genome-integrating and non-integrating. Oncoretroviruses and lentiviruses can integrate into host cellular chromatin while adenoviruses, adeno-associated viruses, and herpes viruses predominantly persist in the cell nucleus as extrachromosomal episomes.

In certain embodiments, a suitable viral vector is a retroviral vector. Retroviruses refer to viruses of the family Retroviridae. Examples of retroviruses include oncoretroviruses, such as murine leukemia virus (MLV), and lentiviruses, such as human immunodeficiency virus 1 (HIV-1). Retroviral genomes are single-stranded (ss) RNAs and comprise various genes that may be provided in cis or trans. For example, retroviral genome may contain cis-acting sequences such as two long terminal repeats (LTR), with elements for gene expression, reverse transcription and integration into the host chromosomes. Other components include the packaging signal (psi or w), for the specific RNA packaging into newly formed virions and the polypurine tract (PPT), the site of the initiation of the positive strand DNA synthesis during reverse transcription. In addition, the retroviral genome may comprise gag, pol and env genes. The gag gene encodes the structural proteins, the pol gene encodes the enzymes that accompany the ssRNA and carry out reverse transcription of the viral RNA to DNA, and the env gene encodes the viral envelope. Generally, the gag, pol and env are provided in trans for viral replication and packaging.

In certain embodiments, a retroviral vector provided herein may be a lentiviral vector. At least five serogroups or serotypes of lentiviruses are recognized. Viruses of the different serotypes may differentially infect certain cell types and/or hosts. Lentiviruses, for example, include primate retroviruses and non-primate retroviruses. Primate retroviruses include HIV and simian immunodeficiency virus (SIV). Non-primate retroviruses include feline immunodeficiency virus (FIV), bovine immunodeficiency virus (BIV), caprine arthritis-encephalitis virus (CAEV), equine infectious anemia virus (EIAV), and visnavirus. Lentiviruses or lentivectors may be capable of transducing quiescent cells. As with oncoretrovirus vectors, the design of lentivectors may be based on the separation of cis- and trans-acting sequences.

In exemplary embodiments, a viral vector provided herein is an adeno-associated virus (AAV). AAV is a small, replication-defective, non-enveloped animal virus that infects humans and some other primate species. AAV is not known to cause human disease and induces a mild immune response. AAV vectors can also infect both dividing and quiescent cells without integrating into the host cell genome.

The AAV genome consists of a linear single stranded DNA which is ~4.7 kb in length. The genome consists of two open reading frames (ORF) flanked by an inverted terminal repeat (ITR) sequence that is about 145 bp in length. The ITR consists of a nucleotide sequence at the 5′ end (5′ ITR) and a nucleotide sequence located at the 3′ end (3′ ITR) that contain palindromic sequences. The ITRs function in cis by folding over to form T-shaped hairpin structures by complementary base pairing that function as primers during initiation of DNA replication for second strand synthesis. The two open reading frames encode for rep and cap genes that are involved in replication and packaging of the virion. In an exemplary embodiment, an AAV vector provided herein does not contain the rep or cap genes. Such genes may be provided in trans for producing virions as described further below.

In certain embodiments, an AAV vector may include a stuffer nucleic acid. In some embodiments, the stuffer nucleic acid may encode a green fluorescent protein or antibiotic resistance gene such as kanamycin or ampicillin. In certain embodiments, the stuffer nucleic acid may be located outside of the ITR sequences (e.g., as compared to the polynucleotide encoding a therapeutic protein, and regulatory sequences, which are located between the 5′ and 3′ ITR sequences).

Various serotypes of AAV exist, including AAV1, AAV2, AAV3, AAV4, AAV5, AAV6, AAV7, AAV8, AAV9, AAV9.47, AAV9 (hul4), AAV10, AAV11, AAV 12, AAV13, AAVrh8, AAVrh10, AAV-DJ, and AAV-DJ8. These serotypes differ in their tropism, or the types of cells they infect. AAVs may comprise the genome and capsids from multiple serotypes (e.g., pseudotypes). For example, an AAV may comprise the genome of serotype 2 (e.g., ITRs) packaged in the capsid from serotype 5 or serotype 9. Pseudotypes may improve transduction efficiency as well as alter tropism.

In some embodiments, an AAV vector or an AAV viral particle, or virion, may be used to deliver a construct comprising a cell selective regulatory element operably linked to a polynucleotide encoding functional therapeutic protein into a cell, cell type, or tissue, and may done either in vivo, ex vivo, or in vitro. In exemplary embodiments, such an AAV vector is replication-deficient. In some embodiments, an AAV virus is engineered or genetically modified so that it can replicate and generate virions only in the presence of helper factors.

In certain embodiments, a viral vector can be selected to produce a virion having high infectivity without selectivity for a particular cell type. In certain embodiments, a viral vector can be designed to produce a virion that infects many different cell types but expression of the transgene is enhanced and/or optimized in a cell type of interest (e.g., PV neurons), and expression of the transgene is reduced and/or minimized in other non-target cell types (e.g., non-PV CNS cells). The differential expression of the transgene in different cell types can be controlled, engineered, or manipulated using different regulatory elements that are selective for one or more cell types. In some cases, one or more regulatory elements operably linked to a polynucleotide encoding a therapeutic protein enhances selective expression of the polynucleotide in a target cell, cell type, or tissue, while the one or more regulatory elements suppress transgene expression in off-target cells, cell type, or tissue, or confers significantly lower, de minimis, or statistically lower gene expression in one or more off-target cells, cell types, or tissue.

In some cases, an AAV serotype that can cross the blood brain barrier or infect cells of the CNS is preferred.

In exemplary embodiments, the application provides expression vectors that have been designed for delivery by an AAV. The AAV can be any serotype, for examples, AAV1, AAV2, AAV3, AAV4, AAV5, AAV6, AAV7, AAV8, AAV9, AAV9.47, AAV9 (hul4), AAV10, AAV11, AAV 12, AAV13, AAVrh8, AAVrh10, AAV-DJ, and AAV-DJ8, or a chimeric, hybrid, or variant AAV. The AAV can also be a self-complementary AAV (scAAV), where a “self-complementary” AAV is one in which the coding region has been designed to form an intra-molecular double-stranded DNA template. Upon infection of such vectors, rather than waiting for cell mediated synthesis of the second strand, the two complementary halves of the scAAV will associate to form one double stranded DNA (dsDNA) unit that is ready for immediate replication and transcription. The design of scAAV vectors is described in a variety of publications, including McCarty et al Gene Therapy 2001 8:1248-54.

In certain embodiments, an expression vector designed for delivery by an AAV comprises a 5′ ITR and a 3′ ITR. In certain embodiments, an expression vector designed for delivery by an AAV comprises a 5′ ITR, a promoter, a construct as described above, and a 3′ ITR. In certain embodiments, an expression vector designed for delivery by an AAV comprises a 5′ ITR, an enhancer, a promoter, a construct as described above, and a 3′ ITR.

### Host Cells

In another aspect, the invention relates to a host cell comprising a nucleic acid cassette as described above. Host cells may be a bacterial cell, a yeast cell, an insect cell or a mammalian cell. In an exemplary embodiment, a host cell refers to any cell line that is susceptible to infection by a virus of interest, and amenable to culture in vitro.

In certain embodiments, a host cell provided herein may be used for ex vivo gene therapy purposes. In such embodiments, the cells are transfected with a nucleic acid molecule or expression cassette as described above subsequently transplanted into the patient or subject. Transplanted cells can have an autologous, allogenic, or heterologous origin. For clinical use, cell isolation will generally be carried out under Good Manufacturing Practices (GMP) conditions. Before transplantation, cell quality and absence of microbial or other contaminants is typically checked and preconditioning, such as with radiation and/or an immunosuppressive treatment, may be carried out. Furthermore, the host cells may be transplanted together with growth factors to stimulate cell proliferation and/or differentiation.

In certain embodiments, a host cell may be used for ex vivo gene therapy into the CNS. Preferably, said cells are eukaryotic cells such as mammalian cells, these include, but are not limited to, humans, non-human primates (such as apes, chimpanzees, monkeys, and orangutans), domesticated animals (including dogs and cats), as well as livestock (such as horses, cattle, pigs, sheep, and goats), or other mammalian species including, without limitation, mice, rats, guinea pigs, rabbits, hamsters, and the like. A person skilled in the art will choose the more appropriate cells according to the patient or subject to be transplanted.

In certain embodiments, a host cell provided herein may be a cell with self-renewal and pluripotency properties, such as stem cells or induced pluripotent stem cells. Stem cells are preferably mesenchymal stem cells. Mesenchymal stem cells (MSCs) are capable of differentiating into at least one of an osteoblast, a chondrocyte, an adipocyte, or a myocyte and may be isolated from any type of tissue. Generally, MSCs will be isolated from bone marrow, adipose tissue, umbilical cord, or peripheral blood. Methods for obtaining thereof are well known to a person skilled in the art. Induced pluripotent stem cells (also known as iPS cells or iPSCs) are a type of pluripotent stem cell that can be generated directly from adult cells. Yamanaka et al. induced iPS cells by transferring the Oct3/4, Sox2, Klf4 and c-Myc genes into mouse and human fibroblasts, and forcing the cells to express the genes (WO 2007/069666). Thomson et al. subsequently produced human iPS cells using Nanog and Lin28 in place of Klf4 and c-Myc (WO 2008/118820).

In an exemplary embodiment, a host cell provided herein is a packaging cell. Said cells can be adherent or suspension cells. The packaging cell, and helper vector or virus or DNA construct(s) provide together in trans all the missing functions which are required for the complete replication and packaging of the viral vector.

Preferably, said packaging cells are eukaryotic cells such as mammalian cells, including simian, human, dog, and rodent cells. Examples of human cells are PER.C6 cells (WO01/38362), MRC-5 (ATCC CCL-171), WI-38 (ATCC CCL-75), HEK-293 cells (ATCC CRL-1573), HeLa cells (ATCC CCL2), and fetal rhesus lung cells (ATCC CL-160). Examples of non-human primate cells are Vero cells (ATCC CCL81), COS-1 cells (ATCC CRL-1650), or COS-7 cells (ATCC CRL-1651). Examples of dog cells are MDCK cells (ATCC CCL-34). Examples of rodent cells are hamster cells, such as BHK21-F, HKCC cells, or CHO cells.

As an alternative to mammalian sources, cell lines for use in the invention may be derived from avian sources such as chicken, duck, goose, quail, or pheasant. Examples of avian cell lines include avian embryonic stem cells (WO01/85938 and WO03/076601), immortalized duck retina cells (WO2005/042728), and avian embryonic stem cell derived cells, including chicken cells (WO2006/108846) or duck cells, such as EB66 cell line (WO2008/129058 & WO2008/142124).

In another embodiment, said host cell are insect cells, such as SF9 cells (ATCC CRL-1711), Sf21 cells (IPLB-Sf21), MG1 cells (BTI-TN-MG1), or High Five™ cells (BTI-TN-5B1-4).

In certain embodiments, the host cells provided herein comprise a nucleic acid construct (e.g., a plasmid) carrying the recombinant AAV vector/genome containing a cassette as described above may further comprise one or more additional nucleic acid constructs, such as, for example (i) a nucleic acid construct (e.g., an AAV helper plasmid) that encodes rep and cap genes, but does not carry ITR sequences; and/or (ii) a nucleic acid construct (e.g., a plasmid) providing the adenoviral functions necessary for AAV replication. In an exemplary embodiment, a host cell provided herein comprises: i) a nucleic acid construct or an expression vector as described above; ii) a nucleic acid construct encoding AAV rep and cap genes which does not carry the ITR sequences; and iii) a nucleic acid construct comprising adenoviral helper genes (as described further below).

In certain embodiments, the rep, cap, and adenoviral helper genes can be combined on a single plasmid (Blouin V et al. J Gene Med. 2004; 6 (suppl): S223-S228; Grimm D. et al. Hum. Gene Ther. 2003; 7:839-850). Thus, in another exemplary embodiment, a host cell provided herein comprises: i) a nucleic acid molecule or an expression cassette and ii) a plasmid encoding AAV rep and cap genes which does not carry the ITR sequences and further comprising adenoviral helper genes. Alternative methods are known. For example, the rep, cap, and adenoviral helper genes do not need to be on the same plasmid can be provide on different plasmids, or the rep and cap genes can be provided on a different plasmid to the adenorviral helper genes.

In certain embodiments, a host cell suitable for large-scale production of AAV vectors is an insect cells that can be infected with a combination of recombinant baculoviruses (Urabe et al. Hum. Gene Ther. 2002; 13:1935-1943). For example, SF9 cells may be co-infected with three baculovirus vectors respectively expressing AAV rep, AAV cap and the AAV vector to be packaged. The recombinant baculovirus vectors will provide the viral helper gene functions required for virus replication and/or packaging.

Further guidance for the construction and production of virions for gene therapy according to the invention can be found in: Viral Vectors for Gene Therapy, Methods and Protocols. Series: Methods in Molecular Biology, Vol. 737. Merten and Al-Rubeai (Eds.); 2011 Humana Press (Springer); Gene Therapy. M. Giacca. 2010 Springer-Verlag; Heilbronn R. and Weger S. Viral Vectors for Gene Transfer: Current Status of Gene Therapeutics. In: Drug Delivery, Handbook of Experimental Pharmacology 197; M. Schafer-Korting (Ed.). 2010 Springer-Verlag; pp. 143-170; Adeno-Associated Virus: Methods and Protocols. R. O. Snyder and P. Moulllier (Eds). 2011 Humana Press (Springer); Bunning H. et al. Recent developments in adeno-associated virus technology. J. Gene Med. 2008; 10:717-733; and Adenovirus: Methods and Protocols. M. Chillon and A. Bosch (Eds.); Third. Edition. 2014 Humana Press (Springer).

### Virions & Methods of Producing Virions

In certain embodiments, the application provides viral particles comprising a viral vector. The terms “viral particle”, and “virion” are used herein interchangeably and relate to an infectious and typically replication-defective virus particle comprising the viral genome (e.g., the viral expression vector) packaged within a capsid and, as the case may be, e.g., for retroviruses, a lipidic envelope surrounding the capsid. A “capsid” refers to the structure in which the viral genome is packaged. A capsid consists of several oligomeric structural subunits made of proteins. For example, AAV have an icosahedral capsid formed by the interaction of three capsid proteins: VP1, VP2, and VP3. In one embodiment, a virion provided herein is a recombinant AAV virion or rAAV virion obtained by packaging an AAV vector in a protein shell.

In certain embodiments, a recombinant AAV virion provided herein may be prepared by encapsidating an AAV genome derived from a particular AAV serotype in a viral particle formed by natural Cap proteins corresponding to an AAV of the same particular serotype. In other embodiments, an AAV viral particle provided herein comprises a viral vector comprising ITR(s) of a given AAV serotype packaged into proteins from a different serotype. See e.g., Bunning H et al. J Gene Med 2008; 10:717-733. For example, a viral vector having ITRs from a given AAV serotype may be package into: a) a viral particle constituted of capsid proteins derived from a same or different AAV serotype (e.g. AAV2 ITRs and AAV9 capsid proteins; AAV2 ITRs and AAV8 capsid proteins; etc.); b) a mosaic viral particle constituted of a mixture of capsid proteins from different AAV serotypes or mutants (e.g. AAV2 ITRs with AAV1 and AAV9 capsid proteins); c) a chimeric viral particle constituted of capsid proteins that have been truncated by domain swapping between different AAV serotypes or variants (e.g. AAV2 ITRs with AAV8 capsid proteins with AAV9 domains); or d) a targeted viral particle engineered to display selective binding domains, enabling stringent interaction with target cell specific receptors (e.g. AAV5 ITRs with AAV9 capsid proteins genetically truncated by insertion of a peptide ligand; or AAV9 capsid proteins non-genetically modified by coupling of a peptide ligand to the capsid surface).

The skilled person will appreciate that an AAV virion provided herein may comprise capsid proteins of any AAV serotype. In one embodiment, the viral particle comprises capsid proteins from an AAV serotype selected from the group consisting of an AAV1, an AAV2, an AAV5, an AAV8, and an AAV9, which are more suitable for delivery to the CNS (M. Hocquemiller et al., Hum Gene Ther 27 (7): 478-496 (2016)). In a particular embodiment, the viral particle comprises a nucleic acid construct of the invention wherein the 5′ITR and 3′ITR sequences of the nucleic acid construct are of an AAV2 serotype and the capsid proteins are of an AAV9 serotype.

Numerous methods are known in the art for production of rAAV virions, including transfection, stable cell line production, and infectious hybrid virus production systems which include adenovirus-AAV hybrids, herpesvirus-AAV hybrids (Conway, J E et al., (1997) J. Virology 71(11):8780-8789) and baculovirus-AAV hybrids. rAAV production cultures for the production of rAAV virus particles all require; 1) suitable host cells, including, for example, human-derived cell lines such as HeLa, A549, or 293 cells, or insect-derived cell lines such as SF-9, in the case of baculovirus production systems; 2) suitable helper virus function, provided by wild-type or mutant adenovirus (such as temperature sensitive adenovirus), herpes virus, baculovirus, or a plasmid construct providing helper functions; 3) AAV rep and cap genes and gene products; 4) a transgene flanked by AAV ITR sequences; and 5) suitable media and media components to support rAAV production.

In various embodiments, the host cells described herein comprise the following three components: (1) a rep gene and a cap gene, (2) genes providing helper functions, and (3) a transgene flanked by ITRs. The AAV rep gene, AAV cap gene, and genes providing helper functions can be introduced into the cell by incorporating said genes into a vector such as, for example, a plasmid, and introducing said vector into the host cell. The rep, cap and helper function genes can be incorporated into the same plasmid or into different plasmids. In a preferred embodiment, the AAV rep and cap genes are incorporated into one plasmid and the genes providing helper functions are incorporated into another plasmid. The various plasmids for creation of a host cell for virion production (e.g., comprising AAV rep and cap genes, helper functions, or a transgene) can be introduced into the cell by using any suitable method well known in the art. Examples of transfection methods include, but are not limited to, co-precipitation with calcium phosphate, DEAE-dextran, polybrene, electroporation, microinjection, liposome-mediated fusion, lipofection, retrovirus infection and biolistic transfection. In certain embodiments, the plasmids providing the rep and cap genes, the helper functions and the transgene can be introduced into the cell simultaneously. In another embodiment, the plasmids providing the rep and cap genes and the helper functions can be introduced in the cell before or after the introduction of plasmid comprising the transgene. In an exemplary embodiment, the cells are transfected simultaneously with three plasmids (e.g., a triple transfection method): (1) a plasmid comprising the transgene, (2) a plasmid comprising the AAV rep and cap genes, and (3) a plasmid comprising the genes providing the helper functions. Exemplary host cells may be 293, A549, or HeLa cells.

In other embodiments, one or more of (1) the AAV rep and cap genes, (2) genes providing helper functions, and (3) the transgene (e.g., a PV selective regulatory element operably linked to a polynucleotide encoding a therapeutic protein disclosed herein), may be carried by the packaging cell, either episomally and/or integrated into the genome of the packaging cell. In one embodiment, host cells may be packaging cells in which the AAV rep and cap genes and helper functions are stably maintained in the host cell and the host cell is transiently transfected with a plasmid containing a transgene. In another embodiment, host cells are packaging cells in which the AAV rep and cap genes are stably maintained in the host cell and the host cell is transiently transfected with a plasmid containing a transgene and a plasmid containing the helper functions. In another embodiment, host cells may be packaging cells in which the helper functions are stably maintained in the host cell and the host cell is transiently transfected with a plasmid containing a transgene and a plasmid containing rep and cap genes. In another embodiment, host cells may be producer cell lines that are stably transfected with rep and cap genes, helper functions and the transgene sequence. Exemplary packaging and producer cells may be derived from 293, A549, or HeLa cells.

In another embodiment, the producer cell line is an insect cell line (typically Sf9 cells) that is infected with baculovirus expression vectors that provide Rep and Cap proteins. This system does not require adenovirus helper genes (Ayuso E, et al., Curr. Gene Ther. 2010, 10:423-436).

The term “cap protein”, as used herein, refers to a polypeptide having at least one functional activity of a native AAV Cap protein (e.g., VP1, VP2, VP3). Examples of functional activities of cap proteins include the ability to induce formation of a capsid, facilitate accumulation of single-stranded DNA, facilitate AAV DNA packaging into capsids (i.e., encapsidation), bind to cellular receptors, and facilitate entry of the virion into host cells. In principle, any Cap protein can be used in the context of the present invention.

Cap proteins have been reported to have effects on host tropism, cell, tissue, or organ specificity, receptor usage, infection efficiency, and immunogenicity of AAV viruses. Accordingly, an AAV cap for use in an rAAV may be selected taking into consideration, for example, the subject's species (e.g., human or non-human), the subject's immunological state, the subject's suitability for long or short-term treatment, or a particular therapeutic application (e.g., treatment of a particular disease or disorder, or delivery to particular cells, tissues, or organs). In certain embodiments, the cap protein is derived from the AAV of the group consisting of AAV1, AAV2, AAV5, AAV8, and AAV9 serotypes. In an exemplary embodiment, the cap protein is derived from AAV9.

In some embodiments, an AAV Cap for use in the method of the invention can be generated by mutagenesis (i.e., by insertions, deletions, or substitutions) of one of the aforementioned AAV caps or its encoding nucleic acid. In some embodiments, the AAV cap is at least 70%, 75%, 80%, 85%, 90%, 95%, 98%, or 99% or more similar to one or more of the aforementioned AAV caps.

In some embodiments, the AAV cap is chimeric, comprising domains from two, three, four, or more of the aforementioned AAV caps. In some embodiments, the AAV cap is a mosaic of VP1, VP2, and VP3 monomers originating from two or three different AAV or a recombinant AAV. In some embodiments, a rAAV composition comprises more than one of the aforementioned caps.

In some embodiments, an AAV cap for use in a rAAV virion is engineered to contain a heterologous sequence or other modification. For example, a peptide or protein sequence that confers selective targeting or immune evasion may be engineered into a cap protein. Alternatively or in addition, the cap may be chemically modified so that the surface of the rAAV is polyethylene glycolated (i.e., pegylated), which may facilitate immune evasion. The cap protein may also be mutagenized (e.g., to remove its natural receptor binding, or to mask an immunogenic epitope).

The term “rep protein”, as used herein, refers to a polypeptide having at least one functional activity of a native AAV rep protein (e.g. rep 40, 52, 68, 78). Examples of functional activities of a rep protein include any activity associated with the physiological function of the protein, including facilitating replication of DNA through recognition, binding and nicking of the AAV origin of DNA replication as well as DNA helicase activity. Additional functions include modulation of transcription from AAV (or other heterologous) promoters and site-specific integration of AAV DNA into a host chromosome. In a particular embodiment, AAV rep genes may be from the serotypes AAV1, AAV2, AAV4, AAV5, AAV6, AAV7, AAV8, AAV9, AAV10, or AAVrh10; more preferably from an AAV serotype selected from the group consisting of AAV1, AAV2, AAV5, AAV8, and AAV9.

In some embodiments, an AAV rep protein for use in the method of the invention can be generated by mutagenesis (i.e., by insertions, deletions, or substitutions) of one of the aforementioned AAV reps or its encoding nucleic acid. In some embodiments, the AAV rep is at least 70%, 75%, 80%, 85%, 90%, 95%, 98%, or 99% or more similar to one or more of the aforementioned AAV reps.

The expressions “helper functions” or “helper genes”, as used herein, refer to viral proteins upon which AAV is dependent for replication. The helper functions include those proteins required for AAV replication including, without limitation, those proteins involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus. Helper functions include, without limitation, adenovirus E1, E2a, VA, and E4 or herpesvirus UL5, ULB, UL52, and UL29, and herpesvirus polymerase. In a preferred embodiment, the proteins upon which AAV is dependent for replication are derived from adenovirus.

In some embodiments, a viral protein upon which AAV is dependent for replication for use in the method of the invention can be generated by mutagenesis (i.e., by insertions, deletions, or substitutions) of one of the aforementioned viral proteins or its encoding nucleic acid. In some embodiments, the viral protein is at least 70%, 75%, 80%, 85%, 90%, 95%, 98%, or 99% or more similar to one or more of the aforementioned viral proteins.

Methods for assaying the functions of cap proteins, rep proteins and viral proteins upon which AAV is dependent for replication are well known in the art.

Host cells for expressing a transgene of interest may be grown under conditions adequate for assembly of the AAV virions. In certain embodiments, host cells are grown for a suitable period of time in order to promote the assembly of the AAV virions and the release of virions into the media. Generally, cells may be grown for about 24 hours, about 36 hours, about 48 hours, about 72 hours, about 4 days, about 5 days, about 6 days, about 7 days, about 8 days, about 9 days, or up to about 10 days. After about 10 days (or sooner, depending on the culture conditions and the particular host cell used), the level of production generally decreases significantly. Generally, time of culture is measured from the point of viral production. For example, in the case of AAV, viral production generally begins upon supplying helper virus function in an appropriate host cell as described herein. Generally, cells are harvested about 48 to about 100, preferably about 48 to about 96, preferably about 72 to about 96, preferably about 68 to about 72 hours after helper virus infection (or after viral production begins).

rAAV production cultures can be grown under a variety of conditions (over a wide temperature range, for varying lengths of time, and the like) suitable to the particular host cell being utilized. rAAV production cultures include attachment-dependent cultures which can be cultured in suitable attachment-dependent vessels such as, for example, roller bottles, hollow fiber filters, microcarriers, and packed-bed or fluidized-bed bioreactors. rAAV vector production cultures may also include suspension-adapted host cells such as HeLa, 293, and SF-9 cells which can be cultured in a variety of ways including, for example, spinner flasks, stirred tank bioreactors, and disposable systems such as the Wave bag system.

Suitable media known in the art may be used for the production of rAAV virions. These media include, without limitation, media produced by Hyclone Laboratories and JRH including Modified Eagle Medium (MEM), Dulbecco's Modified Eagle Medium (DMEM), each of which is incorporated herein by reference in its entirety. In certain embodiments, rAAV production culture media may be supplemented with serum or serum-derived recombinant proteins at a level of 0.5%-20% (v/v or w/v). Alternatively, rAAV vectors may be produced in serum-free conditions which may also be referred to as media with no animal-derived products.

After culturing the host cells to allow AAV virion production, the resulting virions may then be harvested and purified. In certain embodiments, the AAV virions can be obtained from (1) the host cells of the production culture by lysis of the host cells, and/or (2) the culture medium of said cells after a period of time post-transfection, preferably 72 hours. The rAAV virions may be harvested from the spent media from the production culture, provided the cells are cultured under conditions that cause release of rAAV virions into the media from intact cells (see e.g., U.S. Pat. No. 6,566,118). Suitable methods of lysing cells are also known in the art and include for example multiple freeze/thaw cycles, sonication, microfluidization, and treatment with chemicals, such as detergents and/or proteases.

After harvesting, the rAAV virions may be purified. The term “purified” as used herein includes a preparation of rAAV virions devoid of at least some of the other components that may also be present where the rAAV virions naturally occur or are initially prepared from. Thus, for example, purified rAAV virions may be prepared using an isolation technique to enrich it from a source mixture, such as a culture lysate or production culture supernatant. Enrichment can be measured in a variety of ways, such as, for example, by the proportion of DNase-resistant particles (DRPs) or genome copies (gc) present in a solution, or by infectivity, or it can be measured in relation to a second, potentially interfering substance present in the source mixture, such as contaminants, including production culture contaminants or in-process contaminants, including helper virus, media components, and the like.

In certain embodiments, the rAAV production culture harvest may be clarified to remove host cell debris. In some embodiments, the production culture harvest may be clarified using a variety of standard techniques, such as, centrifugation or filtration through a filter of 0.2 μm or greater pore size (e.g., a cellulose acetate filter or a series of depth filters).

In certain embodiments, the rAAV production culture harvest is further treated with Benzonase™ to digest any high molecular weight DNA present in the production culture. In some embodiments, the Benzonase™ digestion is performed under standard conditions, for example, a final concentration of 1-2.5 units/ml of Benzonase™ at a temperature ranging from ambient to 37° C. for a period of 30 minutes to several hours.

In certain embodiments, the rAAV virions may be isolated or purified using one or more of the following purification steps: equilibrium centrifugation; flow-through anionic exchange filtration; tangential flow filtration (TFF) for concentrating the rAAV particles; rAAV capture by apatite chromatography; heat inactivation of helper virus; rAAV capture by hydrophobic interaction chromatography; buffer exchange by size exclusion chromatography (SEC); nanofiltration; and rAAV capture by anionic exchange chromatography, cationic exchange chromatography, or affinity chromatography. These steps may be used alone, in various combinations, or in different orders. Methods to purify rAAV particles are found, for example, in Xiao et al., (1998) Journal of Virology 72:2224-2232; U.S. Pat. Nos. 6,989,264 and 8,137,948; and WO 2010/148143.

In certain embodiments, purified AAV virions can be dialyzed against PBS, filtered and stored at −80° C. Titers of viral genomes can be determined by quantitative PCR using linearized plasmid DNA as standard curve (see e.g., Lock M, et al., Hum. Gene Ther. 2010; 21:1273-1285).

### Pharmaceutical Compositions

In certain embodiments, the application provides compositions comprising a nucleic acid cassette, e.g., an expression cassette, e.g., an rAAV comprising an expression cassette, described above or an RNA, e.g., an mRNA or ncRNA, encoded by the same, and a pharmaceutically acceptable carrier. (It is noted that the RNA may be synthetically produced, and thus is not necessarily generated via transcription of a nucleic acid cassette described herein.) In some embodiments, a virion containing the cassette and a pharmaceutically acceptable carrier is provided. In exemplary embodiments, such compositions are suitable for gene therapy applications. Pharmaceutical compositions are preferably sterile and stable under conditions of manufacture and storage. Sterile solutions may be accomplished, for example, by filtration through sterile filtration membranes.

Acceptable carriers and excipients in the pharmaceutical compositions are preferably nontoxic to recipients at the dosages and concentrations employed. Acceptable carriers and excipients may include buffers such as phosphate, citrate, HEPES, and TAE, antioxidants such as ascorbic acid and methionine, preservatives such as hexamethonium chloride, octadecyldimethylbenzyl ammonium chloride, resorcinol, and benzalkonium chloride, proteins such as human serum albumin, gelatin, dextran, and immunoglobulins, hydrophilic polymers such as polyvinylpyrrolidone, amino acids such as glycine, glutamine, histidine, and lysine, and carbohydrates such as glucose, mannose, sucrose, and sorbitol. Pharmaceutical compositions of the disclosure can be administered parenterally in the form of an injectable formulation. Pharmaceutical compositions for injection can be formulated using a sterile solution or any pharmaceutically acceptable liquid as a vehicle. Pharmaceutically acceptable vehicles include, but are not limited to, sterile water and physiological saline.

The pharmaceutical compositions of the disclosure may be prepared in microcapsules, such as hydroxylmethylcellulose or gelatin-microcapsules and polymethylmethacrylate microcapsules. The pharmaceutical compositions of the disclosure may also be prepared in other drug delivery systems such as liposomes, albumin microspheres, microemulsions, nano-particles, and nanocapsules. The pharmaceutical composition for gene therapy can be in an acceptable diluent, or can comprise a slow release matrix in which the gene delivery vehicle is imbedded.

Pharmaceutical compositions provided herein may be formulated for parenteral administration, subcutancous administration, intravenous administration, intramuscular administration, intra-arterial administration, intraparenchymal administration, intrathecal administration, intra-cisterna magna administration, intracerebroventricular administration, or intraperitoneal administration. The pharmaceutical composition may also be formulated for, or administered via, nasal, spray, oral, aerosol, rectal, or vaginal administration. In one embodiment, a pharmaceutical composition provided herein is administered to the CNS or cerebral spinal fluid (CSF), e.g., by intraparenchymal injection, intrathecal injection, intra-cisterna magna injection, or intracerebroventricular injection. The tissue target may be specific, for example the CNS, or it may be a combination of several tissues, for example the muscle and CNS tissues. Exemplary tissue or other targets may include liver, skeletal muscle, heart muscle, adipose deposits, kidney, lung, vascular endothelium, epithelial, hematopoietic cells, cancer cells, CNS, and/or CSF. In a preferred embodiment, a pharmaceutical composition provided herein is administered to the CNS or CSF by injection, e.g., by intraparenchymal injection, intrathecal injection, intra-cisterna magna injection, or intracerebroventricular injection. One or more of these methods may be used to administer a pharmaceutical composition of the disclosure.

In certain embodiments, a pharmaceutical composition provided herein comprises an “effective amount” or a “therapeutically effective amount.” As used herein, such amounts refer to an amount effective, at dosages and for periods of time necessary to achieve the desired therapeutic result.

The dosage of the pharmaceutical compositions of the disclosure depends on factors including the route of administration, the disease to be treated, and physical characteristics (e.g., age, weight, general health) of the subject. Dosage may be adjusted to provide the optimum therapeutic response. Typically, a dosage may be an amount that effectively treats the disease without inducing significant toxicity. In certain embodiments, the pharmaceutical composition may be formed in a unit dose as needed.

Pharmaceutical compositions of the disclosure may be administered to a subject in need thereof, as medically necessary. In an exemplary embodiment, a single administration is sufficient. In one embodiment, the pharmaceutical composition is suitable for use in human subjects and is administered by intraparenchymal injection, intrathecal injection, intra-cisterna magna injection, or intracerebroventricular injection. In one embodiment, the pharmaceutical composition is delivered via a peripheral vein by bolus injection. In other embodiments, the pharmaceutical composition is delivered via a peripheral vein by infusion.

In another aspect, the application further provides a kit comprising a nucleic acid molecule, vector, host cell, virion, or pharmaceutical composition as described herein in one or more containers. A kit may include instructions or packaging materials that describe how to administer a nucleic acid molecule, vector, host cell, or virion contained within the kit to a patient. Containers of the kit can be of any suitable material, e.g., glass, plastic, metal, etc., and of any suitable size, shape, or configuration. In certain embodiments, the kits may include one or more ampoules or syringes that contain a nucleic acid molecule, vector, host cell, virion, or pharmaceutical composition in a suitable liquid or solution form.

### Methods of Treatment

The present nucleic acid cassette, expression cassette, expression vector, viral vector, viral particle, or pharmaceutical composition may be used for the treatment of a variety of disorders, e.g., neurological disorders. In some embodiments, a chemical, protein, or nucleic acid molecule of the invention may be used to treat or ameliorate one or more symptoms associated with a mutation in a gene, or an under-expressed or non-expressed gene in a subject. In certain embodiments, the treatment may be treating a subject via gene therapy wherein the gene therapy is administered directly to the subject (e.g., directly to the CNS) of a subject in need thereof or systematically via injection and/or infusion. The therapy may be formulated for parenteral administration, subcutaneous administration, intravenous administration, intramuscular administration, intra-arterial administration, intraparenchymal administration, intrathecal administration, intra-cisterna magna administration, intracerebroventricular administration, or intraperitoneal administration, or via, nasal, spray, oral, aerosol, rectal, or vaginal administration, e.g., by intraparenchymal injection, intrathecal injection, intra-cisterna magna injection, or intracerebroventricular injection. The tissue target may be specific, for example the CNS, or it may be a combination of several tissues.

In any embodiment herein, the target cells may be neural cells, muscle cells, cardiac cells, skin cells, immune cells, hematopoetic cells, cancer cells, pancreatic cells, or kidney cells. In some cases, the target cells may be neural cells, e.g., cerebrum cells, brainstem cells, hippocampus cells, or cerebellum cells. For example, in some embodiments, the neural cells are GABAergic cells, e.g., parvalbumin expressing cells. In some cases, the target cell may be a CNS cell, such as an excitatory neuron, a dopaminergic neuron, a glial cell, an ependymal cell, an oligodendrocyte, an astrocyte, a microglia, a motor neuron, a vascular cell, a GABAergic neuron, or a non-GABAergic neuron (e.g., a cell that does not express one or more of GAD2, GAD1, NKX2.1, DLX1, DLX5, SST and VIP), a non-PV neuron (e.g., a GABAergic neuron that does not express parvalbumin), or other CNS cells (e.g., CNS cell types that have never expressed any of PV, GAD2, GAD1, NKX2.1, DLX1, DLX5, SST and VIP).

In any embodiment, the present therapy may be used to increase the production or expression of a target protein in a cell, such as a GABA neuron or a parvalbumin neuron.

In certain embodiments, a treatment provided herein does not result in an adverse reaction for the subject. Treatment with a nucleic acid molecule, expression vector, pharmaceutical composition, or virion described herein can cause fewer, or less severe, adverse reactions in a subject than treatment with a similar gene therapy containing the same transgene linked to a non-parvalbumin neuron-selective regulatory element.

### Sequence Table

Table 4 below provides certain sequences that may be referred to in other parts of this disclosure.

SEQ ID NOS: 1-10 and 43-48 are DRG de-targeting sequences that may be in an RNA transcript encoded in a nucleic acid cassette of the present disclosure.

SEQ ID NOS 57-62, 64-71, 110, and 112 are liver de-targeting sequences that may be in an RNA transcript encoded in a nucleic acid cassette of the present disclosure.

Table 4 also provides the DNA versions of the RNA sequences (which sequences may be in the cassette itself) as well the sequences of certain miRNAs and pre-miRNAs that may bind to those RNA sequences.

Additional sequences, including the sequences of controls and the sequences of some exemplary combinations of sequences that de-target expression in both DRG and liver cells are also listed in Table 4.

TABLE 4

| SEQ | | Detargeted | |
|---|---|---|---|
| ID | Description | Tissue | Sequence (5′ TO 3′) |
| 1 | hsa-mir-196b- | DRG | CCCAACAACAGGAAACUACCUA |
| | 5p binding | | |
| | site; Sequence | | |
| | in transcript | | |
| 2 | hsa-mir-10b- | DRG | CACAAAUUCGGUUCUACAGGGUA |
| | 5p binding | | |
| | site; Sequence | | |
| | in transcript | | |
| 3 | hsa-mir-24-2- | DRG | CUGUGUUUCAGCUCAGUAGGCA |
| | 5p binding | | |
| | site; Sequence | | |
| | in transcript | | |
| 4 | hsa-mir-183- | DRG | UUAUGGCCCUUCGGUAAUUCAC |
| | 3p binding | | |
| | site; Sequence | | |
| | in transcript | | |
| 5 | hsa-mir-196a- | DRG | CCCAACAACAUGAAACUACCUA |
| | 5p binding | | |
| | site; Sequence | | |
| | in transcript | | |
| 6 | hsa-mir-494- | DRG | GAGGUUUCCCGUGUAUGUUUCA |
| | 3p binding | | |
| | site; Sequence | | |
| | in transcript | | |
| 7 | 3′ UTR Tile; | DRG | AAAAGCAAAUGCUGACUGCUGCUCACGCCCUGGCUGU |
| | Sequence in | | GGAUGCCAAAAACUUACUCGAUGUCAUUGACCAAGCA |
| | transcript | | AGACUGAAAAUGCUUGGGCAGACGAGACCACACUGAG |
| | | | CCUCCCCUAGGAGCACGUCUUGCUACCCUCUUUUGAA |
| | | | GAUGUUCUCUAGCCUUCCACCAGCAGCGAGGAAUUAA |
| | | | CCCUGUGUCCUCAGUCGCCAGCACUUACAGCUCCAAC |
| | | | UUU |
| 8 | internal | DRG | UUACUCGAUGUCAUUGACCAAGCAAGACUGAAAAUGC |
| | stretch of | | UUGGGCAGACGAGACCACACUGAGCCUCCCCUAGGAG |
| | SEQ 7; | | CACGUCUUGCUACCCUCUUUUGAAGAUGUUCUCUAGC |
| | Sequence in | | CUUCCACCAGCAGC |
| | transcript | | |
| 9 | 3′ UTR Tile; | DRG | UCUGGAUGGAAUAACUCUUCUUUUGAUGAUGAUCAUA |
| | Sequence in | | AAAUUAAAUUUCAGACUUCACUAGUUCUGCGUAACAC |
| | transcript | | GACAGGUUGCCUAAGGAACCUCAGCUCCGCGGGGGAA |
| | | | GAAGCUCGGAAGCAAAUGCGGUCCUGCGAGGGGCUGG |
| | | | UAGACUCACUGUUGUAUGUGAUCCACACGUGUGUGAA |
| | | | CACAUCCGAUUACGACAGCAAGACGGUGGAGAACUGC |
| | | | GUG |
| 10 | internal | DRG | GACUUCACUAGUUCUGCGUAACACGACAGGUUGCCUA |
| | stretch of | | AGGAACCUCAGCUCCGCGGGGGAAGAAGCUCGGAAGC |
| | SEQ 9; | | AAAUGCGGUCCUGCGAGGGGCUGGUAGACUCACUGUU |
| | Sequence in | | GUAUGUGAUCCACA |
| | transcript | | |
| 11 | hsa-mir-196b- | DRG | CCCAACAACAGGAAACTACCTA |
| | 5p binding | | |
| | site; Sequence | | |
| | in vector | | |
| 12 | hsa-mir-10b- | DRG | CACAAATTCGGTTCTACAGGGTA |
| | 5p binding | | |
| | site; Sequence | | |
| | in vector | | |
| 13 | hsa-mir-24-2- | DRG | CTGTGTTTCAGCTCAGTAGGCA |
| | 5p binding | | |
| | site; Sequence | | |
| | in vector | | |
| 14 | hsa-mir-183- | DRG | TTATGGCCCTTCGGTAATTCAC |
| | 3p binding | | |
| | site; Sequence | | |
| | in vector | | |
| 15 | hsa-mir-196a- | DRG | CCCAACAACATGAAACTACCTA |
| | 5p binding | | |
| | site; Sequence | | |
| | in vector | | |
| 16 | hsa-mir-494- | DRG | GAGGTTTCCCGTGTATGTTTCA |
| | 3p binding | | |
| | site; Sequence | | |
| | in vector | | |
| 17 | 3′ UTR Tile; | DRG | AAAAGCAAATGCTGACTGCTGCTCACGCCCTGGCTGT |
| | Sequence in | | GGATGCCAAAAACTTACTCGATGTCATTGACCAAGCA |
| | vector | | AGACTGAAAATGCTTGGGCAGACGAGACCACACTGAG |
| | | | CCTCCCCTAGGAGCACGTCTTGCTACCCTCTTTTGAA |
| | | | GATGTTCTCTAGCCTTCCACCAGCAGCGAGGAATTAA |
| | | | CCCTGTGTCCTCAGTCGCCAGCACTTACAGCTCCAAC |
| | | | TTT |
| 18 | internal | DRG | TTACTCGATGTCATTGACCAAGCAAGACTGAAAATGC |
| | stretch of | | TTGGGCAGACGAGACCACACTGAGCCTCCCCTAGGAG |
| | SEQ 17; | | CACGTCTTGCTACCCTCTTTTGAAGATGTTCTCTAGC |
| | Sequence in | | CTTCCACCAGCAGC |
| | vector | | |
| 19 | 3′ UTR Tile; | DRG | TCTGGATGGAATAACTCTTCTTTTGATGATGATCATA |
| | Sequence in | | AAATTAAATTTCAGACTTCACTAGTTCTGCGTAACAC |
| | vector | | GACAGGTTGCCTAAGGAACCTCAGCTCCGCGGGGGAA |
| | | | GAAGCTCGGAAGCAAATGCGGTCCTGCGAGGGGCTGG |
| | | | TAGACTCACTGTTGTATGTGATCCACACGTGTGTGAA |
| | | | CACATCCGATTACGACAGCAAGACGGTGGAGAACTGC |
| | | | GTG |
| 20 | internal | DRG | GACTTCACTAGTTCTGCGTAACACGACAGGTTGCCTA |
| | stretch of | | AGGAACCTCAGCTCCGCGGGGGAAGAAGCTCGGAAGC |
| | SEQ 19; | | AAATGCGGTCCTGCGAGGGGCTGGTAGACTCACTGTT |
| | Sequence in | | GTATGTGATCCACA |
| | vector | | |
| 21 | hsa-mir-196b- | | UAGGUAGUUUCCUGUUGUUGGG |
| | 5p | | |
| 22 | hsa-mir-10b- | | UACCCUGUAGAACCGAAUUUGUG |
| | 5p | | |
| 23 | hsa-mir-24-2- | | UGCCUACUGAGCUGAAACACAG |
| | 5p | | |
| 24 | hsa-mir-183- | | GUGAAUUACCGAAGGGCCAUAA |
| | 3p | | |
| 25 | hsa-mir-196a- | | UAGGUAGUUUCAUGUUGUUGGG |
| | 5p | | |
| 26 | hsa-mir-494- | | UGAAACAUACACGGGAAACCUC |
| | 3p | | |
| 27 | hsa-mir-196b | | acuggucggugauuUAGGUAGUUUCCUGUUGUUGGGa |
| | pre-miRNA | | uccaccuuucucUCGACAGCACGACACUGCCUUCauu |
| | | | acuucaguug |
| 28 | hsa-mir-10b | | ccagagguuguaacquugucuauauaUACCCUGUAGA |
| | pre-miRNA | | ACCGAAUUUGUGugguauccguauagucACAGAUUCG |
| | | | AUUCUAGGGGAAUauauggucgaugcaaaaacuuca |
| 29 | hsa-mir-24-2 | | cucugccucccgUGCCUACUGAGCUGAAACACAGuug |
| | pre-miRNA | | guuuguguacacUGGCUCAGUUCAGCAGGAACAGgg |
| 30 | hsa-mir-183 | | ccgcagagugugacuccuguucugugUAUGGCACUGG |
| | pre-miRNA | | UAGAAUUCACUgugaacagucucagucaGUGAAUUAC |
| | | | CGAAGGGCCAUAAacagagcagagacagauccacga |
| 31 | hsa-mir-196a- | | gugaauUAGGUAGUUUCAUGUUGUUGGGccuggguuu |
| | 1 pre-miRNA | | cugaacaCAACAACAUUAAACCACCCGAuucac |
| 32 | hsa-mir-196a- | | ugcucgcucagcugaucuguggcuUAGGUAGUUUCAU |
| | 2 pre-miRNA | | GUUGUUGGGauugaguuuugaacuCGGCAACAAGAAA |
| | | | CUGCCUGAGuuacaucagucgguuuucgucgagggc |
| 33 | hsa-mir-494 | | gauacucgaaggagAGGUUGUCCGUGUUGUCUUCUCU |
| | pre-miRNA | | uuauuuaugaUGAAACAUACACGGGAAACCUCuuuuu |
| | | | uaguauc |
| 34 | 5′ AAV ITR; | | CCTGCAGGCAGCTGCGCGCTCGCTCGCTCACTGAGGC |
| | Sequence in | | CGCCCGGGCGTCGGGCGACCTTTGGTCGCCCGGCCTC |
| | vector | | AGTGAGCGAGCGAGCGCGCAGAGAGGGAGTGGCCAAC |
| | | | TCCATCACTAGGGGTTCCT |
| 35 | 3′ AAV ITR; | | AGGAACCCCTAGTGATGGAGTTGGCCACTCCCTCTCT |
| | Sequence in | | GCGCGCTCGCTCGCTCACTGAGGCCGGGCGACCAAAG |
| | vector | | GTCGCCCGACGCCCGGGCTTTGCCCGGGCGGCCTCAG |
| | | | TGAGCGAGCGAGCGCGCAGCTGCCTGCAGG |
| 36 | hsa-mir-196b- | | UCGACAGCACGACACUGCCUUC |
| | 3p | | |
| 37 | hsa-mir-10b- | | ACAGAUUCGAUUCUAGGGGAAU |
| | 3p | | |
| 38 | hsa-mir-24-2- | | UGGCUCAGUUCAGCAGGAACAG |
| | 3p | | |
| 39 | hsa-mir-183- | | UAUGGCACUGGUAGAAUUCACU |
| | 5p | | |
| 40 | hsa-mir-196a- | | CAACAACAUUAAACCACCCGA |
| | 1-3p | | |
| 41 | hsa-mir-196a- | | CGGCAACAAGAAACUGCCUGAG |
| | 2-3p | | |
| 42 | hsa-mir-494- | | AGGUUGUCCGUGUUGUCUUCUCU |
| | 5p | | |
| 43 | 4xSEQ ID | DRG | CCCAACAACAGGAAACUACCUAUGGCGAGACCUCCAC |
| | NO: 1; | | CCCUCCUCCCAACAACAGGAAACUACCUAGUUGAUAA |
| | Sequence in | | GUCGGGGACAAUCUCCCAACAACAGGAAACUACCUAG |
| | transcript | | CCGUCAUGAACGCACCAGUCCCCCAACAACAGGAAAC |
| | | | UACCUA |
| 44 | 4xSEQ ID | DRG | UUAUGGCCCUUCGGUAAUUCACUGGCGAGACCUCCAC |
| | NO: 4; | | CCCUCCUUUAUGGCCCUUCGGUAAUUCACGUUGAUAA |
| | Sequence in | | GUCGGGGACAAUCUUUAUGGCCCUUCGGUAAUUCACG |
| | transcript | | CCGUCAUGAACGCACCAGUCCUUAUGGCCCUUCGGUA |
| | | | AUUCAC |
| 45 | 4xSEQ ID | DRG | CUGUGUUUCAGCUCAGUAGGCAUGGCGAGACCUCCAC |
| | NO: 3; | | CCCUCCUCUGUGUUUCAGCUCAGUAGGCAGUUGAUAA |
| | Sequence in | | GUCGGGGACAAUCUCUGUGUUUCAGCUCAGUAGGCAG |
| | transcript | | CCGUCAUGAACGCACCAGUCCCUGUGUUUCAGCUCAG |
| | | | UAGGCA |
| 46 | 4xSEQ ID | DRG | CACAAAUUCGGUUCUACAGGGUAGGCGAGACCUCCAC |
| | NO: 2; | | CCCUCCUCACAAAUUCGGUUCUACAGGGUAUUGAUAA |
| | Sequence in | | GUCGGGGACAAUCUCACAAAUUCGGUUCUACAGGGUA |
| | transcript | | CCGUCAUGAACGCACCAGUCCCACAAAUUCGGUUCUA |
| | | | CAGGGUA |
| 47 | 2x(SEQ ID | DRG | CUGUGUUUCAGCUCAGUAGGCAUGGCGAGACCUCCAC |
| | NO: 3 + SEQ | | CCCUCCUCACAAAUUCGGUUCUACAGGGUAGUUGAUA |
| | ID NO: 2); | | AGUCGGGGACAAUCUCUGUGUUUCAGCUCAGUAGGCA |
| | Sequence in | | GCCGUCAUGAACGCACCAGUCCCACAAAUUCGGUUCU |
| | transcript | | ACAGGGUA |
| 48 | 2x(SEQ ID | DRG | CCCAACAACAGGAAACUACCUAUGGCGAGACCUCCAC |
| | NO: 1 + SEQ | | CCCUCCUCACAAAUUCGGUUCUACAGGGUAUUGAUAA |
| | ID NO: 2); | | GUCGGGGACAAUCUACCCAACAACAGGAAACUACCUA |
| | Sequence in | | CCGUCAUGAACGCACCAGUCCUCACAAAUUCGGUUCU |
| | transcript | | ACAGGGUA |
| 49 | 4xSEQ ID | DRG | CCCAACAACAGGAAACTACCTATGGCGAGACCTCCAC |
| | NO: 1; | | CCCTCCTCCCAACAACAGGAAACTACCTAGTTGATAA |
| | Sequence in | | GTCGGGGACAATCTCCCAACAACAGGAAACTACCTAG |
| | vector | | CCGTCATGAACGCACCAGTCCCCCAACAACAGGAAAC |
| | | | TACCTA |
| 50 | 4xSEQ ID | DRG | TTATGGCCCTTCGGTAATTCACTGGCGAGACCTCCAC |
| | NO: 4; | | CCCTCCTTTATGGCCCTTCGGTAATTCACGTTGATAA |
| | Sequence in | | GTCGGGGACAATCTTTATGGCCCTTCGGTAATTCACG |
| | vector | | CCGTCATGAACGCACCAGTCCTTATGGCCCTTCGGTA |
| | | | ATTCAC |
| 51 | 4xSEQ ID | DRG | CTGTGTTTCAGCTCAGTAGGCATGGCGAGACCTCCAC |
| | NO: 3; | | CCCTCCTCTGTGTTTCAGCTCAGTAGGCAGTTGATAA |
| | Sequence in | | GTCGGGGACAATCTCTGTGTTTCAGCTCAGTAGGCAG |
| | vector | | CCGTCATGAACGCACCAGTCCCTGTGTTTCAGCTCAG |
| | | | TAGGCA |
| 52 | 4xSEQ ID | DRG | CACAAATTCGGTTCTACAGGGTAGGCGAGACCTCCAC |
| | NO: 2; | | CCCTCCTCACAAATTCGGTTCTACAGGGTATTGATAA |
| | Sequence in | | GTCGGGGACAATCTCACAAATTCGGTTCTACAGGGTA |
| | vector | | CCGTCATGAACGCACCAGTCCCACAAATTCGGTTCTA |
| | | | CAGGGTA |
| 53 | 2x(SEQ ID | DRG | CTGTGTTTCAGCTCAGTAGGCATGGCGAGACCTCCAC |
| | NO: 3 + SEQ | | CCCTCCTCACAAATTCGGTTCTACAGGGTAGTTGATA |
| | ID NO: 2); | | AGTCGGGGACAATCTCTGTGTTTCAGCTCAGTAGGCA |
| | Sequence in | | GCCGTCATGAACGCACCAGTCCCACAAATTCGGTTCT |
| | vector | | ACAGGGTA |
| 54 | 2x(SEQ ID | DRG | CCCAACAACAGGAAACTACCTATGGCGAGACCTCCAC |
| | NO: 1 + SEQ | | CCCTCCTCACAAATTCGGTTCTACAGGGTATTGATAA |
| | ID NO: 2); | | GTCGGGGACAATCTACCCAACAACAGGAAACTACCTA |
| | Sequence in | | CCGTCATGAACGCACCAGTCCTCACAAATTCGGTTCT |
| | vector | | ACAGGGTA |
| 55 | Random 1 | | GCACCTGTCTAAAAGGCACTATTCCCCATACAATGGT |
| | Sequence in | | CCAAAGTCTGGCAGTTACCATCCGACCGCGTTTTCTG |
| | vector | | CGGAGCCCAGGATACTTTCTTACTTAAGCTTGTGACG |
| | | | GCGGTAGAGCATGAAA |
| 56 | Random 2 | | TGCGTGAGTGTTGGGAATAATAGCTAAGAGGGGCACA |
| | Sequence in | | AGCGAGATCAGAAGCGCAAGTGCTACGGAGCCTATCT |
| | vector | | TTATTGTACTGAGTCTACTATAGGCGACGGGTACGIT |
| | | | ACAAGACTATCATAGC |
| 57 | hsa-mir-22-3p | Liver | ACAGUUCUUCAACUGGCAGCUU |
| | binding site; | | |
| | Sequence in | | |
| | transcript | | |
| 58 | hsa-mir-1258 | Liver | CCACGACCUAAUCCUAACU |
| | binding site; | | |
| | Sequence in | | |
| | transcript | | |
| 59 | hsa-mir-5589- | Liver | GGGAGCUAGGUUGCCAUGUGC |
| | 3p binding | | |
| | site; Sequence | | |
| | in transcript | | |
| 60 | hsa-mir-17-5p | Liver | CUACCUGCACUGUAAGCACUUUG |
| | binding site; | | |
| | Sequence in | | |
| | transcript | | |
| 61 | hsa-mir-203a- | Liver | CUAGUGGUCCUAAACAUUUCAC |
| | 3p binding | | |
| | site; Sequence | | |
| | in transcript | | |
| 62 | hsa-mir-122- | Liver | AUUUAGUGUGAUAAUGGCGU |
| | 3p binding | | |
| | site; Sequence | | |
| | in transcript | | |
| 63 | hsa-mir-122- | Liver | CAAACACCAUUGUCACACUCCA |
| | 5p binding | | |
| | site; Sequence | | |
| | in transcript | | |
| 64 | hsa-mir-93-5p | Liver | CUACCUGCACGAACAGCACUUUG |
| | binding site; | | |
| | Sequence in | | |
| | transcript | | |
| 65 | hsa-mir-19a- | Liver | UCAGUUUUGCAUAGAUUUGCACA |
| | 3p binding | | |
| | site; Sequence | | |
| | in transcript | | |
| 66 | 3′ UTR Tile; | Liver | GAGAUCAUAUGCAUAUGUGUAGGGCUGGAGCGUGUGU |
| | Sequence in | | GUGUCUUGAGAUUGUGUGUGUUGCAGUCAUCAUAUCU |
| | transcript | | AUGUGUUACAGAUUGUGUAUGUUAGCCUUGUGUAUGU |
| | | | GUGCUUGAUUGAGGUGGUGUAUUUGGGUUGAAAUUGU |
| | | | GUCAUAUGUGUGUGCUAUCCAUCUCGUGU |
| 67 | 3′ UTR Tile; | Liver | GCAGAAGAGGUAAGGGAGUGAGGCACUCCUAUCCCAG |
| | Sequence in | | UCUCCCAGGUUUGGUUGAGGGCUCCCCAAGGCAGGGC |
| | transcript | | AAGAUAGCGGCCCUGUCACUGACCCUGGCCUGUGGUG |
| | | | GUCUGAGCUGGGGAGGGAAGGACACCAAUGAAUCAGC |
| | | | UUGGGACCUCUUUAGGCCUUCCCCUUUUCCUC |
| 68 | 3′UTR Tile; | Liver | GCAUUAAAACACACAUACAAACAAAAUCAAAAACACU |
| | Sequence in | | GCGGACUUUCACUCAAGCUGGUCUUUCUUCCCCAGUG |
| | transcript | | UAAGGCAAUCCUGCCUACUAACAACACCAACAACAAA |
| | | | ACACUCCAUCUGUGAAAGCUGACGCAGUUAAGGGGGC |
| | | | UAGGCAGGGCAUUUGUGCCAACUAAGAAUCACCAGAU |
| | | | ACCCACCAUAAGUACCUAUCGCAGUUUUGAAGUCGUU |
| | | | UCUCCC |
| 69 | 3′UTR Tile; | Liver | CAACCAUCACGUCAGGCUGCCCAUCCAAUAGACUCCU |
| | Sequence in | | GGGAUGGGGCAGCCAACCCUGGCUCAUCUCAUCUGCC |
| | transcript | | GCUUGGUGCGUGUGCGUGUGCGUGCAUGUGCGUGUGC |
| | | | GUGUGUGCAGGGGUGAGAAUCUGGCAGAUGGUGCCUC |
| | | | UGCCUGCUCUUCUUCGCCUCCUUUAUUUA |
| 70 | 3′UTR Tile; | Liver | UGGCCCUGCGCAUGCUGAAAUAACUGGAACCCAGCCU |
| | Sequence in | | CUCCUCCUACACCGGCCUACCCAUCUGGGCCCAAGAG |
| | transcript | | CUGCACUCACACUCCUACAACGAAGGACAAACUGUCC |
| | | | AGGUCGGAGGGAUCACGAGACACAGAACCUGGAGGGG |
| | | | UGUG |
| 71 | 3′UTR Tile; | Liver | UCUGACUUGAGAGCUCCCCCAGUCAGAUCUCAGGCUU |
| | Sequence in | | GUCCCCCUGUCAGCUGCCUCCAGAAGGGAAGGUAGCC |
| | transcript | | AGUGCCUGAGAAGACAGUCCCUUUUCUACCCACCGCA |
| | | | CUCCAUAACCUCCAUCUUCUCCCACACUGAUGGCGAG |
| | | | CAGCCCCUGAGCACUUUCUGGGACUGGGAGACUGCUU |
| | | | GGUGUUCCCUGAGGACAAGAGACAUCCUGACAGUGUU |
| | | | GGGCA |
| 72 | hsa-mir-22-3p | Liver | ACAGTTCTTCAACTGGCAGCTT |
| | binding site; | | |
| | Sequence in | | |
| | vector | | |
| 73 | hsa-mir-1258 | Liver | CCACGACCTAATCCTAACT |
| | binding site; | | |
| | Sequence in | | |
| | vector | | |
| 74 | hsa-mir-5589- | Liver | GGGAGCTAGGTTGCCATGTGC |
| | 3p binding | | |
| | site; Sequence | | |
| | in vector | | |
| | (used in | | |
| | screen) | | |
| 75 | hsa-mir-17-5p | Liver | CTACCTGCACTGTAAGCACTTTG |
| | binding site; | | |
| | Sequence in | | |
| | vector | | |
| 76 | hsa-mir-203a- | Liver | CTAGTGGTCCTAAACATTTCAC |
| | 3p binding | | |
| | site; Sequence | | |
| | in vector | | |
| 77 | hsa-mir-122- | Liver | ATTTAGTGTGATAATGGCGT |
| | 3p binding | | |
| | site; Sequence | | |
| | in vector | | |
| 78 | hsa-mir-122- | Liver | CAAACACCATTGTCACACTCCA |
| | 5p binding | | |
| | site; Sequence | | |
| | in vector | | |
| 79 | hsa-mir-93-5p | Liver | CTACCTGCACGAACAGCACTTTG |
| | binding site; | | |
| | Sequence in | | |
| | vector | | |
| 80 | hsa-mir-19a- | Liver | TCAGTTTTGCATAGATTTGCACA |
| | 3p binding | | |
| | site; Sequence | | |
| | in vector | | |
| 81 | 3′UTR Tile; | Liver | GAGATCATATGCATATGTGTAGGGCTGGAGCGTGTGT |
| | Sequence in | | GTGTCTTGAGATTGTGTGTGTTGCAGTCATCATATCT |
| | vector | | ATGTGTTACAGATTGTGTATGTTAGCCTTGTGTATGT |
| | | | GTGCTTGATTGAGGTGGTGTATTTGGGTTGAAATTGT |
| | | | GTCATATGTGTGTGCTATCCATCTCGTGT |
| 82 | 3′UTR Tile; | Liver | GCAGAAGAGGTAAGGGAGTGAGGCACTCCTATCCCAG |
| | Sequence in | | TCTCCCAGGTTTGGTTGAGGGCTCCCCAAGGCAGGGC |
| | vector | | AAGATAGCGGCCCTGTCACTGACCCTGGCCTGTGGTG |
| | | | GTCTGAGCTGGGGAGGGAAGGACACCAATGAATCAGC |
| | | | TTGGGACCTCTTTAGGCCTTCCCCTTTTCCTC |
| 83 | 3′UTR Tile; | Liver | GCATTAAAACACACATACAAACAAAATCAAAAACACT |
| | Sequence in | | GCGGACTTTCACTCAAGCTGGTCTTTCTTCCCCAGTG |
| | vector | | TAAGGCAATCCTGCCTACTAACAACACCAACAACAAA |
| | | | ACACTCCATCTGTGAAAGCTGACGCAGTTAAGGGGGC |
| | | | TAGGCAGGGCATTTGTGCCAACTAAGAATCACCAGAT |
| | | | ACCCACCATAAGTACCTATCGCAGTTTTGAAGTCGTT |
| | | | TCTCCC |
| 84 | 3′UTR Tile; | Liver | CAACCATCACGTCAGGCTGCCCATCCAATAGACTCCT |
| | Sequence in | | GGGATGGGGCAGCCAACCCTGGCTCATCTCATCTGCC |
| | vector | | GCTTGGTGCGTGTGCGTGTGCGTGCATGTGCGTGTGC |
| | | | GTGTGTGCAGGGGTGAGAATCTGGCAGATGGTGCCTC |
| | | | TGCCTGCTCTTCTTCGCCTCCTTTATTTA |
| 85 | 3′UTR Tile; | Liver | TGGCCCTGCGCATGCTGAAATAACTGGAACCCAGCCT |
| | Sequence in | | CTCCTCCTACACCGGCCTACCCATCTGGGCCCAAGAG |
| | vector | | CTGCACTCACACTCCTACAACGAAGGACAAACTGTCC |
| | | | AGGTCGGAGGGATCACGAGACACAGAACCTGGAGGGG |
| | | | TGTG |
| 86 | 3′UTR Tile; | Liver | TCTGACTTGAGAGCTCCCCCAGTCAGATCTCAGGCTT |
| | Sequence in | | GTCCCCCTGTCAGCTGCCTCCAGAAGGGAAGGTAGCC |
| | vector | | AGTGCCTGAGAAGACAGTCCCTTTTCTACCCACCGCA |
| | | | CTCCATAACCTCCATCTTCTCCCACACTGATGGCGAG |
| | | | CAGCCCCTGAGCACTTTCTGGGACTGGGAGACTGCTT |
| | | | GGTGTTCCCTGAGGACAAGAGACATCCTGACAGTGTT |
| | | | GGGCA |
| 87 | hsa-mir-22-3p | | AAGCUGCCAGUUGAAGAACUGU |
| 88 | hsa-mir-1258 | | AGUUAGGAUUAGGUCGUGGAA |
| 89 | hsa-mir-5589- | | GCACAUGGCAACCUAGCUCCC |
| | 3p | | |
| 90 | hsa-mir-17-5p | | CAAAGUGCUUACAGUGCAGGUAG |
| 91 | hsa-mir-203a- | | GUGAAAUGUUUAGGACCACUAG |
| | 3p | | |
| 92 | hsa-mir-122- | | AACGCCAUUAUCACACUAAAU |
| | 3p | | |
| 93 | hsa-mir-122- | | UGGAGUGUGACAAUGGUGUUUG |
| | 5p | | |
| 94 | hsa-mir-93-5p | | CAAAGUGCUGUUCGUGCAGGUAG |
| 95 | hsa-mir-19a- | | UGUGCAAAUCUAUGCAAAACUGA |
| | 3p | | |
| 96 | hsa-mir-22 | | ggcugagccgcaguAGUUCUUCAGUGGCAAGCUUUAu |
| | pre-miRNA | | guccugacccagcuaAAGCUGCCAGUUGAAGAACUGU |
| | | | ugcccucugcc |
| 97 | hsa-mir-1258 | | cuguggcuuccacgaccuaauccuaacuccugcgagu |
| | pre-miRNA | | cccuggAGUUAGGAUUAGGUCGUGGAAgccacagga |
| 98 | hsa-mir-5589 | | GGCUGGGUGCUCUUGUGCAGUgagcaaccuacacaac |
| | pre-miRNA | | UGCACAUGGCAACCUAGCUCCCA |
| 99 | hsa-mir-17 | | gucagaauaauguCAAAGUGCUUACAGUGCAGGUAGu |
| | pre-miRNA | | gauaugugcaucuACUGCAGUGAAGGCACUUGUAGca |
| | | | uuauggugac |
| 100 | hsa-mir-203a | | guguuggggacucgcgcgcuggguccAGUGGUUCUUA |
| | pre-miRNA | | ACAGUUCAACAGUUcuguagcgcaauuGUGAAAUGUU |
| | | | UAGGACCACUAGacccggcgggcgcggcgacagcga |
| 101 | hsa-mir-122 | | ccuuagcagagcugUGGAGUGUGACAAUGGUGUUUGu |
| | pre-miRNA | | gucuaaacuaucaAACGCCAUUAUCACACUAAAUAgC |
| | | | uacugcuaggc |
| 102 | hsa-mir-93 | | cugggggcucCAAAGUGCUGUUCGUGCAGGUAGugug |
| | pre-miRNA | | auuacccaaccuACUGCUGAGCUAGCACUUCCCGagc |
| | | | ccccgg |
| 103 | hsa-mir-19a | | gcaguccucuguuAGUUUUGCAUAGUUGCACUACAag |
| | pre-miRNA | | aagaauguaguUGUGCAAAUCUAUGCAAAACUGAugg |
| | | | uggccugc |
| 104 | hsa-mir-22-5p | | AGUUCUUCAGUGGCAAGCUUUA |
| 105 | hsa-mir-5589- | | GGCUGGGUGCUCUUGUGCAGU |
| | 5p | | |
| 106 | hsa-mir-17-3p | | ACUGCAGUGAAGGCACUUGUAG |
| 107 | hsa-mir-203a- | | AGUGGUUCUUAACAGUUCAACAGUU |
| | 5p | | |
| 108 | hsa-mir-93-3p | | ACUGCUGAGCUAGCACUUCCCG |
| 109 | hsa-mir-19a- | | AGUUUUGCAUAGUUGCACUACA |
| | 5p | | |
| 110 | 2x (SEQ ID | Liver | UCAGUUUUGCAUAGAUUUGCACAaaugguccgcacau |
| | NO: 65 + SEQ | | ggcaaccUAGCUCCcgaccgcgCCACGACCUAAUCCU |
| | ID NO: 58 + | | AACUacuuucuuCUACCUGCACUGUAAGCACUUUGua |
| | SEQ ID | | gagcaugaaagcaccugucuaUCAGUUUUGCAUAGAU |
| | NO: 60) | | UUGCACAaaugguccgcacauggcaaccUAGCUCCcg |
| | binding site; | | accgcgCCACGACCUAAUCCUAACUacuuucuuCUAC |
| | Sequence in | | CUGCACUGUAAGCACUUUG |
| | transcript | | |
| 111 | 2x (SEQ ID | DRG + | CCCAACAACAGGAAACUACCUAAUUAUUUAGUGUGAU |
| | NO: 1 + SEQ | Liver | AAUGGCGUUAUCCCAACAACAGGAAACUACCUAGUAU |
| | ID NO: 62) + | | UUAGUGUGAUAAUGGCGUUUUACUCGAUGUCAUUGAC |
| | SEQ ID NO: 8 | | CAAGCAAGACUGAAAAUGCUUGGGCAGACGAGACCAC |
| | + 2x (SEQ ID | | ACUGAGCCUCCCCUAGGAGCACGUCUUGCUACCCUCU |
| | NO: 1 + SEQ | | UUUGAAGAUGUUCUCUAGCCUUCCACCAGCAGCCCCA |
| | ID NO: 62) + | | ACAACAGGAAACUACCUAGGUAUUUAGUGUGAUAAUG |
| | SEQ ID | | GCGUUGUCCCAACAACAGGAAACUACCUAGAUAUUUA |
| | NO: 70; | | GUGUGAUAAUGGCGUUUGGCCCUGCGCAUGCUGAAAU |
| | Sequence in | | AACUGGAACCCAGCCUCUCCUCCUACACCGGCCUACC |
| | transcript | | CAUCUGGGCCCAAGAGCUGCACUCACACUCCUACAAC |
| | | | GAAGGACAAACUGUCCAGGUCGGAGGGAUCACGAGAC |
| | | | ACAGAACCUGGAGGGGUGUGACUAGU |
| 112 | 3x SEQ ID | Liver | UAUUUAGUGUGAUAAUGGCGUUaaugguccUAUUUAG |
| | NO: 62; | | UGUGAUAAUGGCGUUcgaccgcgUAUUUAGUGUGAUA |
| | Sequence in | | AUGGCGUU |
| | transcript | | |
| 113 | 3x internal | Liver | acaccauugucacacuccacaccauugucacacucca |
| | part of SEQ | | caccauugucacacucc |
| | ID NO: 63; | | |
| | Sequence in | | |
| | transcript | | |
| 114 | 2x (SEQ ID | DRG + | TCAGTTTTGCATAGATTTGCACAaatggtccgcacat |
| | NO: 65 + SEQ | Liver | ggcaaccTAGCTCCcgaccgcgCCACGACCTAATCCT |
| | ID NO: 58 + | | AACTactttcttCTACCTGCACTGTAAGCACTTTGta |
| | SEQ ID | | gagcatgaaagcacctgtctaTCAGTTTTGCATAGAT |
| | NO: 60); | | TTGCACAaatggtccgcacatggcaaccTAGCTCCcg |
| | Sequence in | | accgcgCCACGACCTAATCCTAACTactttcttCTAC |
| | vector | | CTGCACTGTAAGCACTTTG |
| 115 | 2x (SEQ ID | DRG + | CCCAACAACAGGAAACTACCTAATTATTTAGTGTGAT |
| | NO: 1 + SEQ | Liver | AATGGCGTTATCCCAACAACAGGAAACTACCTAGTAT |
| | ID NO: 62) + | | TTAGTGTGATAATGGCGTTTTACTCGATGTCATTGAC |
| | SEQ ID NO: 8 | | CAAGCAAGACTGAAAATGCTTGGGCAGACGAGACCAC |
| | + 2x (SEQ ID | | ACTGAGCCTCCCCTAGGAGCACGTCTTGCTACCCTCT |
| | NO: 1 + SEQ | | TTTGAAGATGTTCTCTAGCCTTCCACCAGCAGCCCCA |
| | ID NO: 62) + | | ACAACAGGAAACTACCTAGGTATTTAGTGTGATAATG |
| | SEQ ID | | GCGTTGTCCCAACAACAGGAAACTACCTAGATATTTA |
| | NO: 70; | | GTGTGATAATGGCGTTTGGCCCTGCGCATGCTGAAAT |
| | Sequence in | | AACTGGAACCCAGCCTCTCCTCCTACACCGGCCTACC |
| | vector | | CATCTGGGCCCAAGAGCTGCACTCACACTCCTACAAC |
| | | | GAAGGACAAACTGTCCAGGTCGGAGGGATCACGAGAC |
| | | | ACAGAACCTGGAGGGGTGTGACTAGT |
| 116 | 3x SEQ ID | Liver | TATTTAGTGTGATAATGGCGTTaatggtccTATTTAG |
| | NO: 62; | | TGTGATAATGGCGTTcgaccgcgTATTTAGTGTGATA |
| | Sequence in | | ATGGCGTT |
| | vector | | |
| 117 | 3x internal | Liver | acaccattgtcacactccacaccattgtcacactcca |
| | part of SEQ | | caccattgtcacactcc |
| | ID NO: 63; | | |
| | Sequence in | | |
| | vector | | |
| 118 | hsa-mir-96-5p | DRG | AGCAAAAAUGUGCUAGUGCCAAA |
| | binding site; | | |
| | Sequence in | | |
| | transcript | | |
| 119 | hsa-mir-182- | DRG | AGUGUGAGUUCUACCAUUGCCAAA |
| | 5p binding | | |
| | site; Sequence | | |
| | in transcript | | |
| 120 | hsa-mir-96-5p | DRG | AGCAAAAATGTGCTAGTGCCAAA |
| | binding site; | | |
| | Sequence in | | |
| | vector | | |
| 121 | hsa-mir-182- | DRG | AGTGTGAGTTCTACCATTGCCAAA |
| | 5p binding | | |
| | site; Sequence | | |
| | in vector | | |
| 122 | hsa-mir-96 | | uggccgauUUUGGCACUAGCACAUUUUUGCUuguguc |
| | pre-miRNA | | ucuccgcucugagcAAUCAUGUGCAGUGCCAAUAUGg |
| | | | gaaa |
| 123 | hsa-mir-182 | | gagcugcuugccuccccccguuUUUGGCAAUGGUAGA |
| | pre-miRNA | | ACUCACACUggugagguaacaggauccggUGGUUCUA |
| | | | GACUUGCCAACUAuggggcgaggacucagccggcac |

### Embodiments

Embodiments of the present disclosure include nucleic acid cassettes encoding transgenes that include one or more DRG de-targeting elements, one or more liver de-targeting elements, or both, RNA transcripts derived therefrom, as well as isolated and/or synthetic RNA molecules that include the one or more DRG de-targeting elements, one or more liver de-targeting elements, or both. Embodiments further include methods of making and using such nucleic acid cassettes and RNA molecules, including the use of the same as therapeutics. The embodiments below are meant to further delineate specific aspects of the present disclosure but are not meant to limit its scope.

Embodiment 1. A nucleic acid cassette comprising a therapeutic transgene encoding an RNA transcript, wherein the RNA transcript comprises a sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii).

Embodiment 2. The nucleic acid cassette of embodiment 1, wherein the RNA transcript comprises a sequence of at least 15 contiguous nucleotides of any of SEQ ID NOs. 1-10 and 43-48 that decreases expression in dorsal root ganglion (DRG) cells.

Embodiment 3. The nucleic acid cassette of embodiment 1 or 2, wherein the RNA transcript further comprises a second sequence of (i), (ii), or (iii).

Embodiment 4. The nucleic acid cassette of embodiment 3, wherein the RNA transcript further comprises a third sequence of (i), (ii), or (iii).

Embodiment 5. The nucleic acid cassette of embodiment 4, wherein the RNA transcript further comprises a fourth sequence of (i), (ii), or (iii).

Embodiment 6. The nucleic acid cassette of any one of embodiments 1-5, wherein the RNA transcript comprises two or more copies of a sequence of: (i), (ii), or (iii).

Embodiment 7. The nucleic acid cassette of embodiment 6, wherein the RNA transcript comprises three or more copies of a sequence of: (i), (ii), or (iii).

Embodiment 8. The nucleic acid cassette of embodiment 7, wherein the RNA transcript comprises four or more copies of a sequence of: (i), (ii), or (iii).

Embodiment 9. The nucleic acid cassette of embodiment 8, wherein the RNA transcript comprises five or more copies of a sequence of: (i), (ii), or (iii).

Embodiment 6. The nucleic acid cassette of any one of embodiments 1-5, wherein the sequence of: (i), (ii), or (iii) provides a binding site for one or more of hsa-mir-196b-5p, hsa-mir-10b-5p, hsa-mir-24-2-5p, hsa-mir-183-3p, hsa-mir-196a-5p and hsa-mir-494-3p.

Embodiment 11. The nucleic acid cassette of any one of embodiments 1-10, wherein the RNA transcript is an mRNA, optionally wherein the sequence of (i), (ii), or (iii) is located in one or more of: a 3′ UTR region of the mRNA, a 5′ UTR of the mRNA, or an intron of the mRNA.

Embodiment 12. The nucleic acid cassette of embodiment 11, wherein the sequence of (i), (ii), or (iii) is located in a 3′ UTR region of the mRNA.

Embodiment 13. The nucleic acid cassette of embodiment 11, wherein the sequence of (i), (ii), or (iii) is located in a 5′ UTR region of the mRNA.

Embodiment 14. The nucleic acid cassette of embodiment 11, wherein the sequence of (i), (ii), or (iii) is located in an intron of the mRNA.

Embodiment 15. The nucleic acid cassette of any one of embodiments 1-14, wherein the nucleic acid cassette is non-naturally occurring.

Embodiment 16. The nucleic acid cassette of any one of embodiments 1-15, wherein the nucleic acid cassette comprises a CNS-selective promoter.

Embodiment 17. The nucleic acid cassette of embodiment 16, wherein the CNS selective promoter is selected from the group consisting of: Ca2+/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

Embodiment 18. The nucleic acid cassette of any one of embodiments 1-17, wherein the nucleic acid cassette comprises an enhancer.

Embodiment 19. The nucleic acid cassette of any one of embodiments 1-18, wherein the RNA transcript is a therapeutic RNA transcript for treating a neural disease or disorder, optionally wherein the RNA transcript is an mRNA that encodes a therapeutic protein that is associated with the neural disease or disorder.

Embodiment 20. The nucleic acid cassette of embodiment 19, wherein the neural disease or disorder is Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder.

Embodiment 21. The nucleic acid cassette of embodiment 19 or 20, wherein the therapeutic protein encoded by the mRNA is selected from (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 22. The nucleic acid cassette of any one of embodiments 1-21, wherein:
(a) the RNA transcript comprises a sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii);
(b) the nucleic acid cassette comprises a CNS-selective promoter; and
(c) the RNA transcript is a therapeutic RNA transcript for treating a neural disease or disorder.

Embodiment 23. The nucleic acid cassette of any one of embodiments 1-22, wherein:
(a) the RNA transcript comprises a sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii); and
(b) the nucleic acid cassette comprises a promoter selected from the group consisting of Ca2+/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters; optionally
(c) wherein the therapeutic RNA transcript is an mRNA that encodes a therapeutic protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCNIB, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 24. The nucleic acid cassette of any one of embodiments 1-23, wherein the sequence of (i), (ii), or (iii), results in decreased expression of the RNA transcript in DRG cells as compared to expression of the RNA transcript in DRG cells from an otherwise equivalent RNA transcript without the sequence of (i), (ii), or (iii); when the RNA transcript is an mRNA, the sequence of (i), (ii), or (iii), results in decreased expression of a polypeptide encoded by the mRNA in DRG cells as compared to expression of the polypeptide in DRG cells from an otherwise equivalent mRNA without the sequence of (i), (ii), or (iii).

Embodiment 25. The nucleic acid cassette of embodiment 24, wherein the sequence of (i), (ii), or (iii), result in decreased expression of the RNA transcript and/or the polypeptide encoded by the mRNA in DRG cells at a level that is at least 1.5 fold, at least 2 fold, at least 5 fold, or at least 10 fold as compared to expression of the RNA transcript or the polypeptide in DRG cells from an otherwise equivalent RNA transcript without the sequence of (i), (ii), or (iii).

Embodiment 26. The nucleic acid cassette of embodiment 24 or 25, wherein the sequence of (i), (ii), or (iii), result in decreased expression of the RNA transcript and/or the polypeptide encoded by the mRNA in DRG cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript and/or the polypeptide in DRG cells from an otherwise equivalent RNA transcript without the sequence of (i), (ii), or (iii).

Embodiment 27. The nucleic acid cassette of any one of embodiments 1-26, wherein the sequence of (i), (ii) or (iii), does not result in significantly decreased expression of the RNA transcript in target cells as compared to expression of the RNA transcript in target cells from an otherwise equivalent RNA transcript without the sequence of (i), (ii), or (iii); when the RNA transcript is an mRNA, the sequence of (i), (ii), or (iii), does not result in significantly decreased expression of a polypeptide encoded by the mRNA in target cells as compared to expression of the polypeptide in target cells from an otherwise equivalent mRNA without the sequence of (i), (ii), or (iii).

Embodiment 28. The nucleic acid cassette of embodiment 27, wherein the sequence of (i), (ii) or (iii), does not decrease expression of the RNA transcript and/or the polypeptide encoded thereby (when the RNA transcript is an mRNA) in the target cells as compared to expression of the polypeptide in the target cells from an otherwise equivalent RNA transcript without the sequence of (i), (ii), or (iii).

Embodiment 29. The nucleic acid cassette of embodiment 27, wherein the sequence of (i), (ii), or (iii), result in expression of the RNA transcript and/or the polypeptide encoded thereby in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the polypeptide in target cells from an otherwise equivalent RNA transcript without the sequence of (i), (ii), or (iii).

Embodiment 30. The nucleic acid cassette of any one of embodiments 27-29, wherein the target cells are neural cells.

Embodiment 31. The nucleic acid cassette of embodiment 30, wherein the neural cells are cerebrum cells, brainstem cells, hippocampus cells, or cerebellum cells.

Embodiment 32. The nucleic acid cassette of embodiment 31, wherein the neural cells are GABAergic cells.

Embodiment 33. The nucleic acid cassette of embodiment 32, wherein the GABAergic cells are parvalbumin expressing cells.

Embodiment 34. The nucleic acid cassette of any one of embodiments 1-33, wherein the nucleic acid cassette is a linear construct or vector.

Embodiment 35. The nucleic acid cassette of embodiment 34, wherein the vector is a plasmid.

Embodiment 36. The nucleic acid cassette of embodiment 34, wherein the vector is a viral vector.

Embodiment 37. The nucleic acid cassette of embodiment 36, wherein the viral vector is an adeno-associated virus (AAV) vector.

Embodiment 38. The nucleic acid cassette of embodiment 37, wherein the AAV is AAV1, AAV2, AAV3, AAV4, AAV5, AAV6, AAV7, AAV8, AAV9, or AAV-DJ.

Embodiment 39. The nucleic acid cassette of embodiment 37 or 38, wherein the AAV is an scAAV.

Embodiment 40. The nucleic acid cassette of embodiment 36, wherein the viral vector is a lentiviral vector.

Embodiment 41. An RNA with a sequence encoded by a nucleic acid cassette of any one of embodiments 1-40.

Embodiment 42. A nucleic acid cassette comprising a transgene encoding an RNA transcript, wherein the RNA transcript is a therapeutic RNA transcript, e.g., an mRNA encoding a therapeutic protein, and comprises a binding site for a miRNA selected from mir-196b-5p, mir-10b-5p, mir-24-2-5p, hsa-mir-183-3p, mir-196a-5p, and mir-494-3p, or a complement thereof.

Embodiment 43. The nucleic acid cassette of embodiment 42, comprising binding sites for two or more miRNAs selected from mir-196b-5p, mir-10b-5p, mir-24-2-5p, hsa-mir-183-3p, mir-196a-5p, and mir-494-3p, or a complement thereof.

Embodiment 44. The nucleic acid cassette of embodiment 42, comprising binding sites for three or more miRNAs selected from mir-196b-5p, mir-10b-5p, mir-24-2-5p, hsa-mir-183-3p, mir-196a-5p, and mir-494-3p, or a complement thereof.

Embodiment 45. The nucleic acid cassette of embodiment 42, comprising two binding sites for a miRNA selected from mir-196b-5p, mir-10b-5p, mir-24-2-5p, hsa-mir-183-3p, mir-196a-5p, and mir-494-3p, or a complement thereof.

Embodiment 46. The nucleic acid cassette of embodiment 42, comprising three binding sites for a miRNA selected from mir-196b-5p, mir-10b-5p, mir-24-2-5p, hsa-mir-183-3p, mir-196a-5p, and mir-494-3p, or a complement thereof.

Embodiment 47. The nucleic acid cassette of embodiment 42, comprising four binding sites for a miRNA selected from mir-196b-5p, mir-10b-5p, mir-24-2-5p, hsa-mir-183-3p, mir-196a-5p, and mir-494-3p, or a complement thereof.

Embodiment 48. The nucleic acid cassette of embodiment 42, comprising more than four binding sites for a miRNA selected from mir-196b-5p, mir-10b-5p, mir-24-2-5p, hsa-mir-183-3p, mir-196a-5p, and mir-494-3p, or a complement thereof.

Embodiment 49. The nucleic acid cassette of any one of embodiments 42-48, wherein the miRNA is mir-196b-5p.

Embodiment 50. The nucleic acid cassette of any one of embodiments 42-48, wherein the miRNA is mir-10b-5p.

Embodiment 51. The nucleic acid cassette of any one of embodiments 42-48, wherein the miRNA is mir-24-2-5p.

Embodiment 52. The nucleic acid cassette of any one of embodiments 42-48, wherein the miRNA is hsa-mir-183-3p.

Embodiment 53. The nucleic acid cassette of any one of embodiments 42-48, wherein the miRNA is mir-196a-5p.

Embodiment 54. The nucleic acid cassette of any one of embodiments 42-48, wherein the miRNA is mir-494-3p.

Embodiment 55. The nucleic acid cassette of any one of embodiments 42-48, wherein the RNA transcript comprises multiple copies of the same miRNA binding site.

Embodiment 56. The nucleic acid cassette of any one of embodiments 42-48, wherein the RNA transcript comprises multiple different miRNA binding sites.

Embodiment 57. The nucleic acid cassette of any one of embodiments 42-48, wherein the RNA transcript comprises multiple copies of the same miRNA binding site and multiple different miRNA binding sites.

Embodiment 58. The nucleic acid cassette of any one of embodiments 42-57, wherein the RNA transcript additionally comprises a sequence of at least 10 contiguous nucleotides of any of SEQ ID NOs. 1-10 and 43-48 that decreases expression in DRG cells.

Embodiment 59. The nucleic acid cassette of any one of embodiments 42-57, wherein the RNA transcript additionally comprises at least two sequences of at least 20 contiguous nucleotides of any of SEQ ID NOs. 1-10 and 43-48 that decreases expression in DRG cells.

Embodiment 60. The nucleic acid cassette of any one of embodiments 42-59, wherein the RNA transcript is an mRNA, wherein the miRNA binding site is located in one or more of: a 3′ UTR region of the mRNA, a 5′ UTR of the mRNA or an intron of the mRNA.

Embodiment 61. The nucleic acid cassette of embodiment 60, wherein the miRNA binding site is located in a 3′ UTR region of the mRNA.

Embodiment 62. The nucleic acid cassette of embodiment 60, wherein the miRNA binding site is located in a 5′ UTR region of the mRNA.

Embodiment 63. The nucleic acid cassette of embodiment 60, wherein the miRNA binding site is located in an intron of the mRNA.

Embodiment 64. The nucleic acid cassette of any one of embodiments 42-63, wherein the nucleic acid cassette is non-naturally occurring.

Embodiment 65. The nucleic acid cassette of any one of embodiments 42-64, wherein the nucleic acid cassette comprises a promoter.

Embodiment 66. The nucleic acid cassette of any one of embodiments 42-65, wherein the nucleic acid cassette comprises a CNS selective promoter.

Embodiment 67. The nucleic acid cassette of embodiment 66, wherein the CNS selective promoter is selected from the group consisting of: Ca2+/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

Embodiment 68. The nucleic acid cassette of any one of embodiments 42-67, wherein the nucleic acid cassette comprises an enhancer.

Embodiment 69. The nucleic acid cassette of any one of embodiments 42-68, wherein the therapeutic RNA transcript is for treating a neural disease or disorder, optionally wherein the therapeutic RNA transcript is an mRNA that encodes a therapeutic protein that is associated with a neural disease or disorder.

Embodiment 70. The nucleic acid cassette of embodiment 69, wherein the therapeutic protein is selected from (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCNIB, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 71. The nucleic acid cassette of any one of embodiments 42-70, wherein:
(a) the RNA transcript comprises a sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii);
(b) the nucleic acid cassette comprises a CNS-selective promoter; and
(c) the RNA transcript is a therapeutic RNA transcript for treating a neural disease or disorder.

Embodiment 72. The nucleic acid cassette of embodiment 71, wherein:
(a) the RNA transcript comprises a sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii); and
(b) the nucleic acid cassette comprises a promoter selected from the group consisting of Ca2+/calmodulin-dependent kinase subunit α (CaMKII) promoters, synapsin I promoters, 67 kDa glutamic acid decarboxylase (GAD67) promoters, homcobox DIx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters; optionally
(c) wherein the RNA transcript is an mRNA that encodes a therapeutic protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 73. The nucleic acid cassette of any one of embodiments 42-72, wherein the miRNA binding site results in decreased expression of the RNA transcript and/or a polypeptide encoded thereby (when the RNA transcript is an mRNA) in DRG cells as compared to expression of the polypeptide in DRG cells from an otherwise equivalent RNA transcript without the miRNA binding site.

Embodiment 74. The nucleic acid cassette of any one of embodiments 42-73, wherein the miRNA binding site results in decreased expression of the RNA transcript and/or a polypeptide encoded thereby in DRG cells at a level that is at least 1.5 fold, at least 2 fold, at least 5 fold, or at least 10 fold as compared to expression of the polypeptide in DRG cells from an otherwise equivalent RNA transcript without the miRNA binding site.

Embodiment 75. The nucleic acid cassette of any one of embodiments 42-74, wherein the miRNA binding site results in decreased expression of a polypeptide encoded by the RNA transcript in DRG cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the polypeptide in DRG cells from an otherwise equivalent RNA transcript without the miRNA binding site.

Embodiment 76. The nucleic acid cassette of any one of embodiments 42-75, wherein the sequence of (i), (ii), or (iii), does not result in significantly decreased expression of the RNA transcript in target cells as compared to expression of the RNA transcript in target cells from an otherwise equivalent RNA transcript without the sequence of (i), (ii), or (iii); when the RNA transcript is an mRNA, the sequence of (i), (ii), or (iii), does not result in significantly decreased expression of a polypeptide encoded by the mRNA in target cells as compared to expression of the polypeptide in target cells from an otherwise equivalent mRNA without the sequence of (i), (ii), or (iii).

Embodiment 77. The nucleic acid cassette of embodiment 76, wherein the sequence of (i), (ii), or (iii), does not decrease expression of the RNA transcript in target cells as compared to expression of the RNA transcript in target cells from an otherwise equivalent RNA transcript without the sequence of (i), (ii), or (iii); when the RNA transcript is an mRNA, the sequence of (i), (ii), or (iii), does not decrease expression of polypeptide encoded by the mRNA in the target cells as compared to expression of the polypeptide in the target cells from an otherwise equivalent mRNA without the sequence of (i), (ii), or (iii).

Embodiment 78. The nucleic acid cassette of embodiment 76, wherein the sequence of (i), (ii), or (iii), result in expression of the RNA transcript in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the RNA transcript in target cells from an otherwise equivalent mRNA without the sequence of (i), (ii), or (iii); when the RNA transcript is an mRNA, the sequence of (i), (ii), or (iii), result in expression of a polypeptide encoded by the mRNA in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the polypeptide in target cells from an otherwise equivalent mRNA without the sequence of (i), (ii), or (iii).

Embodiment 79. The nucleic acid cassette of any one of embodiments 42-78, wherein the target cells are neural cells.

Embodiment 80. The nucleic acid cassette of embodiment 79, wherein the neural cells are cerebrum cells, brainstem cells, hippocampus cells or cerebellum cells.

Embodiment 81. The nucleic acid cassette of embodiment 80, wherein the neural cells are GABAergic cells.

Embodiment 82. The nucleic acid cassette of embodiment 81, wherein the GABAergic cells are parvalbumin expressing cells.

Embodiment 83. The nucleic acid cassette of any one of embodiments 42-82, wherein the nucleic acid cassette is a linear construct or a vector.

Embodiment 84. The nucleic acid cassette of embodiment 83, wherein the vector is a plasmid.

Embodiment 85. The nucleic acid cassette of embodiment 83, wherein the vector is a viral vector.

Embodiment 86. The nucleic acid cassette of embodiment 85, wherein the viral vector is an adeno-associated virus (AAV) vector.

Embodiment 87. The nucleic acid cassette of embodiment 86, wherein the AAV is AAV1, AAV2, AAV3, AAV4, AAV5, AAV6, AAV7, AAV8, AAV9, or AAV-DJ.

Embodiment 88. The nucleic acid cassette of embodiment 86 or 87, wherein the AAV is an scAAV.

Embodiment 89. The nucleic acid cassette of embodiment 85, wherein the viral vector is a lentiviral vector.

Embodiment 90. An RNA encoded by a nucleic acid cassette of any one of embodiments 42-89.

Embodiment 91. The nucleic acid cassette of any one of embodiments 42-89, wherein the RNA transcript is an mRNA that encodes a polypeptide.

Embodiment 92. The nucleic acid cassette of embodiment 91, wherein the polypeptide is a therapeutic protein.

Embodiment 93. An RNA with a sequence encoded by the nucleic acid cassette of any one of embodiments 42-92.

Embodiment 94. A method of decreasing dorsal root ganglion (DRG) expression of a therapeutic RNA transcript or a therapeutic protein encoded by the therapeutic RNA transcript (i.e., when the therapeutic RNA transcript is an mRNA) while maintaining expression of the therapeutic RNA transcript or the therapeutic protein in a target tissue, the method comprises including a sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii) in the therapeutic RNA transcript.

Embodiment 95. The method of embodiment 94, wherein the therapeutic RNA transcript further comprises a second sequence of (i), (ii), or (iii).

Embodiment 96. The method of embodiment 94, wherein the therapeutic RNA transcript further comprises a third sequence of (i), (ii), or (iii).

Embodiment 97. The method of embodiment 94, wherein the therapeutic RNA transcript further comprises a fourth sequence of (i), (ii), or (iii).

Embodiment 98. The method of embodiment 94, wherein the therapeutic RNA transcript comprises five or more sequences of (i), (ii), or (iii).

Embodiment 99. The method of any of embodiments 94-98, wherein the therapeutic RNA transcript comprises two or more copies of a sequence of: (i), (ii), or (iii).

Embodiment 100. The method of any of embodiments 94-99, wherein the therapeutic RNA transcript comprises three or more copies of a sequence of: (i), (ii), or (iii).

Embodiment 101. The method of any of embodiments 94-100, wherein the therapeutic RNA transcript comprises four or more copies of a sequence of: (i), (ii), or (iii).

Embodiment 102. The method of any of embodiments 94-101, wherein the therapeutic RNA transcript comprises five or more copies of a sequence of: (i), (ii), or (iii).

Embodiment 103. The method of any of embodiments 94-102, wherein the therapeutic RNA transcript comprises at least 10 contiguous nucleotides of any of SEQ ID NOs. 1-10 and 43-48 that decreases expression in DRG cells.

Embodiment 104. The method of any one of embodiments 94-103, wherein the therapeutic RNA transcript is an mRNA, wherein the sequence of (i), (ii), or (iii) is located in one or more of: a 3′ UTR region of the mRNA, a 5′ UTR of the mRNA or an intron of the mRNA.

Embodiment 105. The method of embodiment 104, wherein the sequence of (i), (ii), or (iii) is located in a 3′ UTR region of the mRNA.

Embodiment 106. The method of embodiment 104, wherein the sequence of (i), (ii), or (iii) is located in a 5′ UTR region of the mRNA.

Embodiment 107. The method of embodiment 104, wherein the sequence of (i), (ii), or (iii) is located in an intron of the mRNA.

Embodiment 108. The method of any one of embodiments 94-107, wherein method comprises administering a nucleic acid cassette encoding the therapeutic RNA transcript to a subject.

Embodiment 109. The method of any one of embodiments 94-108, wherein the administering is systemically administering.

Embodiment 110. The method of any one of embodiments 94-108, wherein the administering is locally administering.

Embodiment 111. The method of embodiment 110, wherein the nucleic acid is administered locally into to the brain or CNS tissue.

Embodiment 112. The method of embodiment 110 or 111, wherein the administering by intraparenchymal, intrathecal, intra-cisterna magna, intracerebroventricular or intracranial administration.

Embodiment 113. The method of any one of embodiments 94-112, wherein the therapeutic RNA transcript is for treating a neural disease or disorder.

Embodiment 114. The method of embodiment 113, wherein the RNA transcript is an mRNA that encodes a therapeutic protein is selected from (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 115. The method of any one of embodiments 108-114, wherein the subject has a neural disease or disorder.

Embodiment 116. The method of embodiment 115, wherein the subject has Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder.

Embodiment 117. The method of any one of embodiments 108-116, wherein the nucleic acid cassette comprises a CNS selective promoter.

Embodiment 118. The method of embodiment 117, wherein the CNS selective promoter is selected from the group consisting of: Ca2+/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

Embodiment 119. The method of any one of embodiments 108-118, wherein:
(a) the therapeutic RNA transcript comprises a sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii);
(b) the nucleic acid cassette comprises a CNS-selective promoter; and
(c) the therapeutic RNA transcript is for treating a neural disease or disorder.

Embodiment 120. The method of embodiment 119, wherein:
(a) the therapeutic RNA transcript comprises a sequence of (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii); and
(b) the nucleic acid cassette comprises a promoter selected from the group consisting of Ca2+/calmodulin-dependent kinase subunit α (CaMKII) promoters, synapsin I promoters, 67 kDa glutamic acid decarboxylase (GAD67) promoters, homeobox DIx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters; optionally
(c) wherein the therapeutic RNA transcript is an mRNA, wherein the mRNA encodes a therapeutic protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 121. The method of any one of embodiments 94-120, wherein the sequence of (i), (ii), or (iii), result in decreased expression of the RNA transcript in DRG cells at a level that is at least 1.5 fold, at least 2 fold, at least 5 fold, or at least 10 fold as compared to expression of the RNA transcript in DRG cells from an otherwise equivalent RNA transcript without the sequence of (i), (ii), or (iii); when the RNA transcript is an mRNA, the sequence of (i), (ii), or (iii), result in decreased expression of the protein encoded by the mRNA in DRG cells at a level that is at least 1.5 fold, at least 2 fold, at least 5 fold, or at least 10 fold as compared to expression of the protein in DRG cells from an otherwise equivalent mRNA without the sequence of (i), (ii), or (iii).

Embodiment 122. The method of any one of embodiments 94-121, wherein the sequence of (i), (ii), or (iii), result in decreased expression of the RNA transcript in DRG cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript in DRG cells from an otherwise equivalent RNA transcript without the sequence of (i), (ii), or (iii); when the RNA transcript is an mRNA, the sequence of (i), (ii), or (iii), result in decreased expression of a protein encoded by the mRNA in DRG cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the protein in DRG cells from an otherwise equivalent mRNA without the sequence of (i), (ii), or (iii).

Embodiment 123. The method of any one of embodiments 94-122, wherein the sequence of (i), (ii), or (iii), does not result in significantly decreased expression of the RNA transcript in target cells as compared to expression of the RNA transcript in target cells from an otherwise equivalent RNA transcript without the sequence of (i), (ii), or (iii); when the RNA transcript is an mRNA, the sequence of (i), (ii), or (iii), does not result in significantly decreased expression of the protein encoded by the mRNA in target cells as compared to expression of the protein in target cells from an otherwise equivalent mRNA without the sequence of (i), (ii), or (iii).

Embodiment 124. The method of embodiment 123, wherein the sequence of (i), (ii), or (iii), does not decrease expression of the RNA transcript and/or the protein encoded by the same (i.e., when the RNA transcript is an mRNA) in the target cells as compared to expression of the RNA transcript and/or protein in the target cells from an otherwise equivalent mRNA without the sequence of (i), (ii), or (iii).

Embodiment 125. The method of any one of embodiments 94-124, wherein the sequence of (i), (ii), or (iii), result in expression of the RNA transcript in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the RNA transcript in target cells from an otherwise equivalent RNA transcript without the sequence of (i), (ii), or (iii); when the RNA transcript is an mRNA, the sequence of (i), (ii), or (iii), result in expression of a protein encoded by the mRNA in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the protein in target cells from an otherwise equivalent mRNA without the sequence of (i), (ii), or (iii).

Embodiment 126. The method of any one of embodiments 124-125, wherein the target cells are neural cells.

Embodiment 127. The method of embodiment 126, wherein the neural cells are cerebrum cells, brainstem cells, hippocampus cells or cerebellum cells.

Embodiment 128. The method of embodiment 127, wherein the neural cells are GABAergic cells.

Embodiment 129. The method of embodiment 128, wherein the GABAergic cells are parvalbumin expressing cells.

Embodiment 130. The method of any one of embodiments 94-129, wherein the RNA transcript is expressed from a nucleic acid cassette.

Embodiment 131. The method of embodiment 130, wherein the nucleic acid cassette is a linear construct.

Embodiment 132. The method of embodiment 130, wherein the nucleic acid cassette is a vector.

Embodiment 133. The method of embodiment 132, wherein the vector is a plasmid.

Embodiment 134. The method of embodiment 132, wherein the vector is a viral vector.

Embodiment 135. The method of embodiment 134, wherein the viral vector is an adeno-associated virus (AAV) vector.

Embodiment 136. The method of embodiment 135, wherein the AAV is AAV1, AAV2, AAV3, AAV4, AAV5, AAV6, AAV7, AAV8, AAV9, or AAV-DJ.

Embodiment 137. The method of embodiment 135 or 136, wherein the AAV is an scAAV.

Embodiment 138. The method of embodiment 134, wherein the viral vector is a lentiviral vector.

Embodiment 139. The method of any one of embodiments 132-138, wherein the method comprises administering the vector to a subject.

Embodiment 140. The method of any one of embodiments 132-138, further comprising administering the vector to a subject.

Embodiment 141.o A nucleic acid cassette comprising a therapeutic transgene encoding an RNA transcript, wherein the RNA transcript comprises a sequence of (i) any SEQ ID NOS. 65, 110, and 112, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii).

Embodiment 142.o The nucleic acid cassette of embodiment 141, wherein the sequence decreases expression of the RNA transcript in liver cells.

Embodiment 143.o The nucleic acid cassette of embodiment 141 or 142, wherein the RNA transcript further comprises a second sequence of (i), (ii), or (iii).

Embodiment 144.o The nucleic acid cassette of embodiment 143, wherein the RNA transcript further comprises a third sequence of (i), (ii), or (iii).

Embodiment 145.o The nucleic acid cassette of embodiment 144, wherein the RNA transcript further comprises a fourth sequence of (i), (ii), or (iii).

Embodiment 146.o The nucleic acid cassette of any one of embodiments 141-145, wherein the RNA transcript comprises two or more copies of a sequence of: (i), (ii), or (iii).

Embodiment 147.o The nucleic acid cassette of embodiment 146, wherein the RNA transcript comprises three or more copies of a sequence of: (i), (ii), or (iii).

Embodiment 148.o The nucleic acid cassette of embodiment 147, wherein the RNA transcript comprises four or more copies of a sequence of: (i), (ii), or (iii).

Embodiment 149.o The nucleic acid cassette of embodiment 148, wherein the RNA transcript comprises five or more copies of a sequence of: (i), (ii), or (iii).

Embodiment 150.o The nucleic acid cassette of any one of embodiments 141 to 149, wherein the RNA transcript is an mRNA, wherein the sequence is located in one or more of: a 3′ UTR region of the mRNA, a 5′ UTR of the mRNA, or an intron of the mRNA.

Embodiment 151.o The nucleic acid cassette of embodiment 150, wherein the sequence is located in a 3′ UTR region of the mRNA.

Embodiment 152.o The nucleic acid cassette of embodiment 150, wherein the sequence is located in a 5′ UTR region of the mRNA.

Embodiment 153.o The nucleic acid cassette of embodiment 150, wherein the sequence is located in an intron of the mRNA.

Embodiment 154.o The nucleic acid cassette of any one of embodiments 141 to 153, wherein the nucleic acid cassette is non-naturally occurring.

Embodiment 155.o The nucleic acid cassette of any one of embodiments 141 to 154, wherein the nucleic acid cassette comprises a CNS-selective promoter.

Embodiment 156.o The nucleic acid cassette of embodiment 155, wherein the CNS selective promoter is selected from the group consisting of: Ca2+/calmodulin-dependent kinase subunit α (CaMKII) promoters, synapsin I promoters, 67 kDa glutamic acid decarboxylase (GAD67) promoters, homeobox DIx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAPIB promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

Embodiment 157.o The nucleic acid cassette of any one of embodiments 141 to 156, wherein the nucleic acid cassette comprises an enhancer.

Embodiment 158.o The nucleic acid cassette of any one of embodiments 141 to 157, wherein the RNA transcript is a therapeutic RNA transcript for treating a neural disease or disorder.

Embodiment 159.o The nucleic acid cassette of embodiment 158, wherein the neural disease or disorder is Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder.

Embodiment 160.o The nucleic acid cassette of embodiment 158 or 159, wherein the therapeutic RNA transcript is an mRNA encoding a therapeutic protein, wherein the therapeutic protein is selected from (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 161.o The nucleic acid cassette of any one of embodiments 141 to 160, wherein:
(a) the RNA transcript comprises the sequence;
(b) the nucleic acid cassette comprises a CNS-selective promoter; and
(c) the RNA transcript is a therapeutic RNA transcript for treating a neural disease or disorder.

Embodiment 162. The nucleic acid cassette of any one of embodiments 141 to 161, wherein:
(a) the RNA transcript comprises the sequence; and
(b) the nucleic acid cassette comprises a promoter selected from the group consisting of Ca2+/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters; optionally
(c) wherein the RNA transcript is an mRNA, wherein the mRNA encodes a therapeutic protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCNIB, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 163. The nucleic acid cassette of any one of embodiments 141 to 162, wherein the sequence results in decreased expression of the RNA transcript and/or a polypeptide encoded by the same (i.e., when the RNA transcript is an mRNA) in liver cells as compared to expression of the RNA transcript and/or polypeptide in liver cells from an otherwise equivalent RNA transcript without the sequence.

Embodiment 164. The nucleic acid cassette of embodiment 163, wherein the sequences results in decreased expression of the RNA transcript and/or a polypeptide encoded by the same in liver cells at a level that is at least 1.5 fold, at least 2 fold, at least 5 fold, or at least 10 fold as compared to expression of the RNA transcript and/or polypeptide in liver cells from an otherwise equivalent RNA transcript without the sequence.

Embodiment 165. The nucleic acid cassette of embodiment 163 or 164, wherein the sequence results in decreased expression of the RNA transcript and/or a polypeptide encoded by the same in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript and/or polypeptide in liver cells from an otherwise equivalent RNA transcript without the sequence.

Embodiment 166. The nucleic acid cassette of any one of embodiments 141 to 165, wherein the sequence does not result in significantly decreased expression of the RNA transcript and/or a polypeptide encoded by the same in target cells as compared to expression of the RNA transcript and/or polypeptide in target cells from an otherwise equivalent RNA transcript without the sequence.

Embodiment 167. The nucleic acid cassette of embodiment 166, wherein the sequence of does not decrease expression of the RNA transcript and/or a polypeptide encoded by the same in the target cells as compared to expression of the RNA transcript and/or polypeptide in the target cells from an otherwise equivalent RNA transcript without the sequence.

Embodiment 168. The nucleic acid cassette of embodiment 167, wherein the sequence result in expression of the RNA transcript and/or a polypeptide encoded by the same in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the RNA transcript and/or polypeptide in target cells from an otherwise equivalent RNA transcript without the sequence.

Embodiment 169. The nucleic acid cassette of any one of embodiments 166-168, wherein the target cells are neural cells.

Embodiment 170. The nucleic acid cassette of embodiment 169, wherein the neural cells are cerebrum cells, brainstem cells, hippocampus cells, or cerebellum cells.

Embodiment 171. The nucleic acid cassette of embodiment 169, wherein the neural cells are GABAergic cells.

Embodiment 172. The nucleic acid cassette of embodiment 170, wherein the GABAergic cells are parvalbumin expressing cells.

Embodiment 173. The nucleic acid cassette of any one of embodiments 141 to 172, wherein the nucleic acid cassette is a linear construct or vector.

Embodiment 174. The nucleic acid cassette of embodiment 173, wherein the vector is a plasmid.

Embodiment 175. The nucleic acid cassette of embodiment 173, wherein the vector is a viral vector.

Embodiment 176. The nucleic acid cassette of embodiment 175, wherein the viral vector is an adeno-associated virus (AAV) vector.

Embodiment 177. The nucleic acid cassette of embodiment 176, wherein the AAV is AAV1, AAV2, AAV3, AAV4, AAV5, AAV6, AAV7, AAV8, AAV9, or AAV-DJ.

Embodiment 178. The nucleic acid cassette of embodiment 176 or 177, wherein the AAV is an scAAV.

Embodiment 179. The nucleic acid cassette of embodiment 173, wherein the viral vector is a lentiviral vector.

Embodiment 180. An RNA with a sequence encoded by a nucleic acid cassette of any one of embodiments 141-180.

Embodiment 181. A method of decreasing liver expression of a therapeutic RNA transcript and/or protein encoded by the same (i.e., when the RNA transcript is an mRNA) while maintaining expression of the RNA transcript and/or protein in a target tissue, the method comprises including a sequence of (i) any SEQ ID NOS. SEQ ID NOS. 65, 110, and 112, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii).

Embodiment 182. The method of embodiment 181, wherein when the RNA transcript is an mRNA, the sequence is located in one or more of: a 3′ UTR region of the mRNA, a 5′ UTR of the mRNA or an intron of the mRNA.

Embodiment 183. The method of embodiment 182, wherein the sequence is located in a 3′ UTR region of the mRNA.

Embodiment 184. The method of embodiment 182, wherein the sequence is located in a 5′ UTR region of the mRNA.

Embodiment 185. The method of embodiment 182, wherein the sequence is located in an intron of the mRNA.

Embodiment 186. The method of any one of embodiments 181-185, wherein method comprises administering a nucleic acid cassette encoding the RNA transcript to a subject.

Embodiment 187. The method of embodiment 186, wherein the administering is systemically administering.

Embodiment 188. The method of embodiment 186, wherein the administering is locally administering.

Embodiment 189. The method of embodiment 188, wherein the nucleic acid is administered locally into to the brain or CNS tissue.

Embodiment 190. The method of embodiment 188 or 189, wherein the administering by intraparenchymal, intrathecal, intra-cisterna magna, intracerebroventricular or intracranial administration.

Embodiment 191. The method of any one of embodiments 181-190, wherein the RNA transcript is a therapeutic RNA transcript for treating a neural disease or disorder.

Embodiment 192. The method of embodiment 191, wherein the therapeutic RNA transcript is an mRNA encoding a therapeutic protein, wherein the therapeutic protein is selected from (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCNIB, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 193. The method of any one of embodiments 186-193, wherein the subject has a neural disease or disorder

Embodiment 194. The method of embodiment 186, wherein the subject has Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder.

Embodiment 195. The method of any one of embodiments 181-194, wherein the nucleic acid cassette comprises a CNS selective promoter.

Embodiment 196. The method of embodiment 195, wherein the CNS selective promoter is selected from the group consisting of: Ca2+/calmodulin-dependent kinase subunit α (CaMKII) promoters, synapsin I promoters, 67 kDa glutamic acid decarboxylase (GAD67) promoters, homeobox DIx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAPIB promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

Embodiment 197. The method of any one of embodiments 181-196, wherein:
(a) the RNA transcript comprises the sequence;
(b) the nucleic acid cassette comprises a CNS-selective promoter; and
(c) the RNA transcript is a therapeutic RNA transcript for treating a neural disease or disorder.

Embodiment 198. The method of any one of embodiments 181-197, wherein:
(a) the RNA transcript comprises the sequence; and
(b) the nucleic acid cassette comprises a promoter selected from the group consisting of Ca2+/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters; optionally
(c) wherein the RNA transcript is an mRNA, the mRNA encodes a therapeutic protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 199. The method of any one of embodiments 181-198, wherein including the sequence results in decreased expression of the RNA transcript and/or a protein encoded by the same (i.e., when the RNA transcript is an mRNA) in liver cells at a level that is at least 1.5 fold, at least 2 fold, at least 5 fold, or at least 10 fold as compared to expression of the RNA transcript and/or protein in liver cells from an otherwise equivalent RNA transcript without the sequence.

Embodiment 200. The method of any one of embodiments 181-199, wherein including the sequence results in decreased expression of the RNA transcript and/or a protein encoded by the same in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript and/or protein in liver cells from an otherwise equivalent RNA transcript without the sequence.

Embodiment 201. The method of any one of embodiments 181-200, wherein the sequence does not result in significantly decreased expression of the RNA transcript and/or a protein encoded by the same in target cells as compared to expression of the RNA transcript and/or protein in target cells from an otherwise equivalent RNA transcript without the sequence.

Embodiment 202. The method of embodiment 201, wherein the sequence does not decrease expression of the RNA transcript and/or a protein encoded by the same in the target cells as compared to expression of the RNA transcript and/or protein in the target cells from an otherwise equivalent RNA transcript without the sequence.

Embodiment 203. The method of any one of embodiments 181-202, wherein the sequence result in expression of the RNA transcript and/or a protein encoded by the same in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the RNA transcript and/or protein in target cells from an otherwise equivalent RNA transcript without the sequence.

Embodiment 204. The method of any one of embodiments 201-203, wherein the target cells are neural cells.

Embodiment 205. The method of embodiment 204, wherein the neural cells are cerebrum cells, brainstem cells, hippocampus cells or cerebellum cells.

Embodiment 206. The method of embodiment 205, wherein the neural cells are GABAergic cells.

Embodiment 207. The method of embodiment 206, wherein the GABAergic cells are parvalbumin expressing cells.

Embodiment 208. The method of any one of embodiments 181-207, wherein the RNA transcript is expressed from a nucleic acid cassette.

Embodiment 209. The method of embodiment 208, wherein the nucleic acid cassette is a linear construct.

Embodiment 210. The method of embodiment 208 or 209, wherein the nucleic acid cassette is a vector.

Embodiment 211. The method of embodiment 210, wherein the vector is a plasmid.

Embodiment 212. The method of embodiment 210, wherein the vector is a viral vector.

Embodiment 213. The method of embodiment 212, wherein the viral vector is an adeno-associated virus (AAV) vector.

Embodiment 214. The method of embodiment 213, wherein the AAV is AAV1, AAV2, AAV3, AAV4, AAV5, AAV6, AAV7, AAV8, AAV9, or AAV-DJ.

Embodiment 215. The method of embodiment 213 or 214, wherein the AAV is an scAAV.

Embodiment 216. The method of embodiment 212, wherein the viral vector is a lentiviral vector.

Embodiment 217. The method of any one of embodiments 210-216, wherein the method comprises administering the vector to a subject.

Embodiment 218. The method of any one of embodiments 181-207, wherein the method comprises administering the RNA transcript to a subject.

Embodiment 219. A nucleic acid cassette comprising a therapeutic transgene encoding an RNA transcript that comprises a first sequence that de-targets expression in dorsal root ganglion (DRG) cells and a second sequence that de-targets expression in liver cells.

Embodiment 220. The nucleic acid cassette of embodiment 219, wherein the first and second sequences result in decreased expression of the RNA transcript or a polypeptide encoded by the same (i.e., when the RNA transcript is an mRNA) in DRG and liver cells relative to a target tissue.

Embodiment 221. The nucleic acid cassette of embodiment 219 or 220, wherein the first and second sequences result in:
decreased expression of the RNA transcript or a polypeptide encoded by the same (i.e., when the RNA transcript is an mRNA) in DRG cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript or polypeptide in DRG cells from an otherwise equivalent RNA transcript without the first and second sequences and, independently,
decreased expression of the RNA transcript or a polypeptide encoded by the same in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript or polypeptide in liver cells from an otherwise equivalent RNA transcript without the first and second sequences.

Embodiment 222. The nucleic acid cassette of any one of embodiments 219-221, wherein:
(a) the first sequence is: (i) any of SEQ ID NOs. 1-10 and 43-48, (ii) a variant, functional fragment, or combination thereof, or (iii) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (i) or (ii); and
(b) the second sequence is: (iv) any of SEQ ID NOs. 57-62, 64-71, 110, and 112, (v) a variant, functional fragment, or combination thereof, or (vi) a sequence at least 80%, 81%, 82%, 83%, 84%, 85%, 86%, 87%, 88%, 89%, 90%, 91%, 92%, 93%, 94%, 95%, 96%, 97%, 98%, or 99% identical to (iv) or (v).

Embodiment 222A: The nucleic acid cassette of any one of embodiments 219-222, wherein the first sequence of: (i), (ii), or (iii) provides a binding site for one or more of hsa-mir-196b-5p, hsa-mir-10b-5p, hsa-mir-24-2-5p, hsa-mir-183-3p, hsa-mir-196a-5p and hsa-mir-494-3p and the second sequence of: (iv), (v), or (vi) provides a binding site for hsa-mir-22-3p, hsa-mir-1258, hsa-mir-5589-3p, hsa-mir-17-5p, hsa-mir-203a-3p, hsa-mir-122-3p, hsa-mir-93-5p, and hsa-mir-19a-3p.

Embodiment 223. The nucleic acid cassette of any one of embodiments 219-222A, wherein:
the first sequence comprises at least 15 contiguous nucleotides of any of SEQ ID NOs. 1-10 and 43-48 that decreases expression in dorsal root ganglion (DRG) cells; and
the second sequence comprises at least 15 contiguous nucleotides of any of SEQ ID NOs. 57-62, 64-71, 110, and 112, that decreases expression in liver cells.

Embodiment 224. The nucleic acid cassette of one of embodiments 219-223, wherein the RNA transcript comprises at least two copies of a sequence of any of (i)-(vi).

Embodiment 225. The nucleic acid cassette of embodiment 224, wherein the RNA transcript comprises at least three or at least four copies of a sequence of any of (i)-(vi).

Embodiment 226. The nucleic acid of any one of embodiments 219-225, wherein the RNA transcript comprises a combination of sequences selected from Table 3.

Embodiment 227. The nucleic acid cassette of one of embodiments 219-227, wherein the RNA transcript is an mRNA, wherein the first and second sequences are independently located in one or more of: a 3′ UTR region of the mRNA, a 5′ UTR of the mRNA, or an intron of the mRNA.

Embodiment 228. The nucleic acid cassette of embodiment 227, wherein the first and second sequences are located in a 3′ UTR region of the mRNA.

Embodiment 229. The nucleic acid cassette of embodiment 227, wherein the first and second sequences are located in a 5′ UTR region of the mRNA.

Embodiment 230. The nucleic acid cassette of embodiment 227, wherein the first and second sequences are located in an intron of the mRNA.

Embodiment 231. The nucleic acid cassette of any one of embodiments 219-230, wherein the nucleic acid cassette is non-naturally occurring.

Embodiment 232. The nucleic acid cassette of one of embodiments 219-231, wherein the nucleic acid cassette comprises a CNS-selective promoter.

Embodiment 233. The nucleic acid cassette of embodiment 232, wherein the CNS selective promoter is selected from the group consisting of: Ca2+/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

Embodiment 234. The nucleic acid cassette of one of embodiments 219-233, wherein the nucleic acid cassette comprises an enhancer.

Embodiment 235. The nucleic acid cassette of any one of embodiments 219-234, wherein the RNA transcript is a therapeutic RNA transcript for treating a neural disease or disorder.

Embodiment 236. The nucleic acid cassette of embodiment 235, wherein the neural disease or disorder is Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder.

Embodiment 237. The nucleic acid cassette of embodiment 235 or 236, wherein the RNA transcript is an mRNA encoding a therapeutic protein, wherein the therapeutic protein is selected from (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 238. The nucleic acid cassette of any one of embodiments 219-237, wherein:
the nucleic acid cassette comprises a CNS-selective promoter; and
the RNA transcript is a therapeutic RNA transcript for treating a neural disease or disorder.

Embodiment 239. The nucleic acid cassette of any one of embodiments 219-238, wherein:
the nucleic acid cassette comprises a promoter selected from the group consisting of Ca2+/calmodulin-dependent kinase subunit α (CaMKII) promoters, synapsin I promoters, 67 kDa glutamic acid decarboxylase (GAD67) promoters, homeobox Dlx5/6 promoters, glutamate receptor 1 (GluR1) promoters, preprotachykinin 1 (Tac1) promoters, Neuron-specific enolase (NSE) promoters, dopaminergic receptor 1 (Drdla) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters; optionally
wherein RNA transcript is an mRNA, wherein the mRNA encodes a therapeutic protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

Embodiment 240. The nucleic acid cassette of any one of embodiments 219-239, wherein the first and second sequences result in decreased expression of the RNA transcript and/or a polypeptide encoded by the same (i.e., when the RNA transcript is an mRNA) in DRG and liver cells as compared to expression of the RNA transcript and/or polypeptide in DRG and liver cells from an otherwise equivalent RNA transcript without the first and second sequences.

Embodiment 241. The nucleic acid cassette of embodiment 240, wherein the first and second sequences result in decreased expression of the RNA transcript and/or a polypeptide encoded by the same in DRG and liver cells at a level that is at least 1.5 fold, at least 2 fold, at least 5 fold, or at least 10 fold as compared to expression of the RNA transcript and/or polypeptide in DRG and liver cells from an otherwise equivalent RNA transcript without the first and second sequences.

Embodiment 242. The nucleic acid cassette of embodiment 240 or 241, wherein the first and second sequences result in decreased expression of the RNA transcript and/or a polypeptide encoded by the same in DRG and liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript and/or polypeptide in DRG and liver cells from an otherwise equivalent RNA transcript without the first and second sequences.

Embodiment 243. The nucleic acid cassette of any one of embodiments 219-242, wherein the first and second sequences do not result in significantly decreased expression of the RNA transcript and/or a polypeptide encoded by the same in target cells as compared to expression of the RNA transcript and/or polypeptide in target cells from an otherwise equivalent RNA transcript without the first and second sequences.

Embodiment 244. The nucleic acid cassette of embodiment 243, wherein the first and second sequences do not decrease expression of the RNA transcript and/or a polypeptide encoded by the same in the target cells as compared to expression of the RNA transcript and/or polypeptide in the target cells from an otherwise equivalent RNA transcript without the first and second sequences.

Embodiment 245. The nucleic acid cassette of any one of embodiments 219-244, wherein the first and second sequences result in expression of the RNA transcript and/or a polypeptide encoded by the same in target cells at a level that is at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the RNA transcript and/or polypeptide in target cells from an otherwise equivalent RNA transcript without the first and second sequences.

Embodiment 246. The nucleic acid cassette of any one of embodiments 243-245, wherein the target cells are neural cells.

Embodiment 247. The nucleic acid cassette of embodiment 246, wherein the neural cells are cerebrum cells, brainstem cells, hippocampus cells, or cerebellum cells.

Embodiment 248. The nucleic acid cassette of embodiment 247, wherein the neural cells are GABAergic cells.

Embodiment 249. The nucleic acid cassette of embodiment 248, wherein the GABAergic cells are parvalbumin expressing cells.

Embodiment 250. The nucleic acid cassette of any one of embodiments 219-249, wherein the nucleic acid cassette is a linear construct or vector.

Embodiment 251. The nucleic acid cassette of embodiment 250, wherein the vector is a plasmid.

Embodiment 252. The nucleic acid cassette of embodiment 250, wherein the vector is a viral vector.

Embodiment 253. The nucleic acid cassette of embodiment 252, wherein the viral vector is an adeno-associated virus (AAV) vector.

Embodiment 254. The nucleic acid cassette of embodiment 253, wherein the AAV is AAV1, AAV2, AAV3, AAV4, AAV5, AAV6, AAV7, AAV8, AAV9, or AAV-DJ.

Embodiment 255. The nucleic acid cassette of embodiment 253 or 254, wherein the AAV is an scAAV.

Embodiment 256. The nucleic acid cassette of embodiment 252, wherein the viral vector is a lentiviral vector.

Embodiment 257. An RNA transcript with a sequence encoded by a nucleic acid cassette of any one of embodiments 219-256.

Embodiment 258. A method of decreasing dorsal root ganglion (DRG) and liver expression of a therapeutic RNA transcript and/or a protein encoded by the same (i.e., when the RNA transcript is an mRNA) while maintaining expression of the RNA transcript and/or protein in a target tissue, the method comprises adding a first and second sequences to the RNA transcript, wherein the first sequence de-targets expression in DRG cells and the second sequence de-targets expression in liver cells.

Embodiment 259. The method of embodiment 219 or 220, wherein the first and second sequences result in:
decreased expression of the RNA transcript and/or a polypeptide encoded by the same in DRG cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript or polypeptide in DRG cells from an otherwise equivalent RNA transcript without the first and second sequences and, independently,
decreased expression of the RNA transcript and/or a polypeptide encoded by the same in liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript or polypeptide in liver cells from an otherwise equivalent RNA transcript without the first and second sequences.

Embodiment 260. The method of embodiment 258 or 258, wherein the RNA transcript is encoded by a nucleic acid cassette of any one of embodiments 219-256.

Embodiment 261. A method for expressing a therapeutic protein, comprising:
administering the nucleic acid cassette of any one of embodiments 219-256 to the subject.

Embodiment 262. The method of embodiment 261, wherein the administering is systemically administering.

Embodiment 263. The method of embodiment 261, wherein the administering is locally administering.

Embodiment 264. The method of embodiment 263, wherein the nucleic acid cassette is administered locally into the brain or CNS tissue.

Embodiment 265. The method of embodiment 263 or 264, wherein the administering by intraparenchymal, intrathecal, intra-cisterna magna, intracerebroventricular or intracranial administration.

Embodiment 266. The method of any of embodiments 261-265, wherein the subject has a neural disease or disorder

Embodiment 267. The method of embodiment 266, wherein the subject has Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder.

Embodiment 268. The method of any one of embodiments 261-267, wherein the RNA transcript and/or a protein encoded by the same (i.e., when the RNA transcript is an mRNA) is expressed in DRG and liver cells at a level that is at least 1.5 fold, at least 2 fold, at least 5 fold, or at least 10 fold as compared to expression of the RNA transcript and/or protein in DRG and liver cells from an otherwise equivalent RNA transcript without the first and second sequences.

Embodiment 269. The method of any one of embodiments 261-268, wherein the RNA transcript and/or a protein encoded by the same is expressed in in DRG and liver cells at a level that is at least 2%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% lower than expression of the RNA transcript and/or protein in DRG and liver cells from an otherwise equivalent RNA transcript without the first and second sequences.

Embodiment 270. The method of any one of embodiments 261-269, wherein expression of the RNA transcript and/or a protein encoded by the same is not decreased in target cells as compared to expression of the RNA transcript and/or protein in target cells from an otherwise equivalent RNA transcript without the first and second sequences.

Embodiment 271. The method of any one of embodiments 261-270, wherein the RNA transcript and/or a protein encoded by the same is not decreased in the target cells as compared to expression of the RNA transcript and/or protein in the target cells from an otherwise equivalent RNA transcript without the first and second sequences.

Embodiment 272. The method of any one of embodiments 261-271, wherein the RNA transcript and/or a protein encoded by the same (i.e., when the RNA transcript is an mRNA) is expressed in target cells at a level that is at least at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% of the expression of the RNA transcript and/or protein in target cells from an otherwise equivalent RNA transcript without the first and second sequences.

Embodiment 273. The method of any one of embodiments 270-272, wherein the target cells are neural cells.

Embodiment 274. The method of embodiment 273, wherein the neural cells are cerebrum cells, brainstem cells, hippocampus cells or cerebellum cells.

Embodiment 275. The method of embodiment 274, wherein the neural cells are GABAergic cells.

Embodiment 276. The method of embodiment 275, wherein the GABAergic cells are parvalbumin expressing cells.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular (ly); i.p., intraperitoneal (ly); s.c., subcutaneous (ly); and the like.

### Example 1

### Screening for DRG De-Targeting Elements

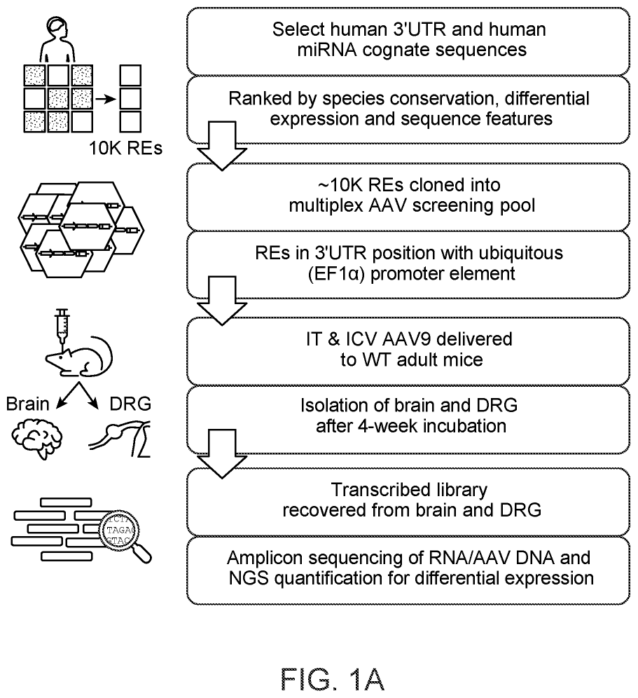

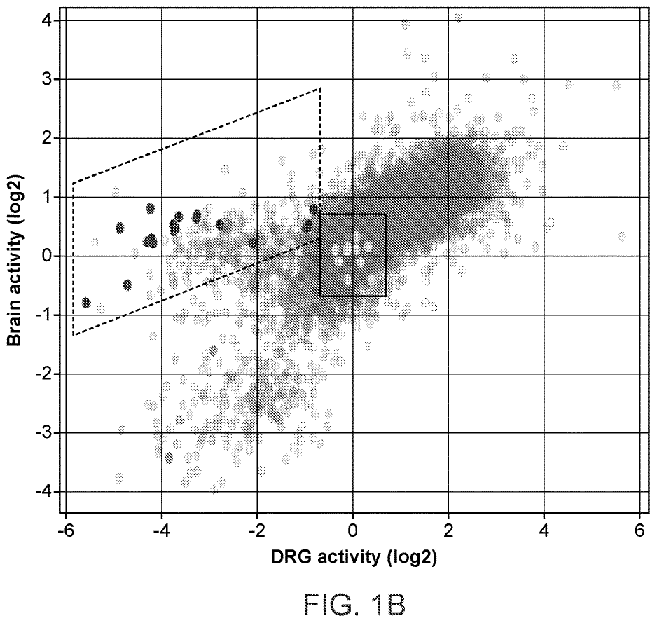

Candidate DRG de-targeting element library preparation: FIG. 1A shows a flow chart of DRG de-targeting regulatory element (RE) library design. Candidate dorsal root ganglion (DRG) de-targeting elements were selected from annotated 3′ untranslated regions (3′ UTR) of genes having the following characteristics: (i) low expression in the DRG; (ii) high expression in neural tissue (e.g., cortex, hippocampus); and (iii) similar expression pattern between human and mouse. The 3′UTRs were screened as discreet overlapping regions (or “tiles”).

Additional candidate DRG de-targeting elements designed to serve as miRNA binding sites were selected based on the following criteria: (i) high expression of the miRNA in DRG; and (ii) low expression in another tissue(s), e.g., other neural tissues including cortex and hippocampus. Seventeen candidate DRG de-targeting miRNA binding sites were included in the screen.

An adeno-associated virus (AAV) genome plasmid library was generated to screen the selected candidate DRG de-targeting elements. The AAV genome plasmid used to generate the library included 5′ and 3′ ITR regions (SEQ ID NOs: 30 and 31, respectively) flanking an expression cassette including the following operably linked elements (in 5′-3′ order): EF1α promoter, EGFP-KASH transgene, de-targeting region with barcodes (described below), WPRE, and a human growth hormone (hGH) polyA signal sequence. The AAV genome plasmid also included restriction enzyme sites positioned to allow for the cloning of candidate DRG de-targeting sequences and associated barcodes into the de-targeting region.

Candidate DRG de-targeting miRNA binding sites were present in the de-targeting region of the library constructs (i) individually, (ii) as 2, 3, or 4 tandem repeats of the same miRNA binding site, and (iii) as combinatorial tetramers of 4 different miRNA binding sites. In constructs (ii) and (iii), miRNA binding sites were separated by a 22 bp spacer sequence. The library contained five replicates of each construct. Each construct, including each of the five replicate constructs, included a unique barcode sequence for identification in downstream analyses. This library contained approximately 10,000 unique combinatorial tetramers (constructs in (iii) above), each of which was approximately 225 bp in length.

Candidate DRG de-targeting elements from 3′ UTRs (or “tiles”) were screened individually, i.e., one candidate DRG de-targeting tile was present in the de-targeting region of the library construct. The library contained five replicates of each construct, with each including a unique barcode sequence for identification in downstream analyses. For cloning, the sequence of each candidate DRG de-targeting tile was checked for the presence of restriction enzyme sites necessary for downstream cloning, and those containing these sites were discarded. Each of the remaining candidate DRG de-targeting tiles were assigned a barcode element and synthesized with flanking sequences for cloning into the AAV genome plasmid backbone (Twist Biosciences).

Representative negative control AAV genome plasmids were also generated, which either lacked a de-targeting region altogether or included a randomized sequence that was not selected as a candidate DRG de-targeting element. These negative control plasmids included all other functional regions of the AAV genome plasmid, including barcodes that allowed their downstream identification as the negative controls.

The candidate DRG de-targeting library, including both the miRNA-based and 3′UTR based de-targeting regions, was transformed into electrocompetent E. coli cells, expanded in 200 mL LB liquid culture media, and harvested for plasmid purification (Qiagen Plasmid Plus Maxi kit) to generate the AAV genome plasmid library preparation. A portion of the transformed E. coli cells were plated on agar plates, allowed to form individual colonies, harvested and Sanger sequenced to validate the fidelity of the cloning process. The negative control AAV genome plasmids were spiked into the AAV genome plasmid library preparation to create the final AAV genome plasmid library used for AAV vector production.

AAV library preparation: AAV9 vectors were produced in adherent HEK293T cells in DMEM +10% FBS according to industry standards. In brief, cells were triple-transfected using PEI-MAX with (i) the candidate DRG de-targeting element AAV genome plasmid library (described above), (ii) Rep/Cap plasmid, and (iii) pALD-X80 (an adenoviral helper plasmid). AAV was harvested from the HEK293T cells and purified from the lysate using an ultra-centrifugation gradient of Iodixanol and further polished with an anion exchange column, followed by concentration and formulation in a PBS-based buffer.

AAV library injection: Time-pregnant female C57BL/6J mice (The Jackson Laboratory) were obtained and housed onsite to acclimate to the new environment until they littered. After birth, pups (n=6) at post-natal day 1 (P1) were injected by intrathecal (IT) injections with the AAV library. Each P1 mouse received 3.74E11 gc/animal in a single 6 μL dose. Pups were fed by their mother and later given a standard chow diet ad libitum and placed on a 12 light/12dark light cycle. Pups were housed together as a litter in a vivarium, weaned at day 21, and sacrificed at 4 weeks of age for sample collection and analyses.

Sample collection: Tissues from injected mice were collected directly into cold RNAlater (Sigma Aldrich). Tissues collected included forebrain, midbrain, hindbrain, spinal cord, and all pairs of L1-L6 dorsal root ganglia (DRG). Samples were kept in RNAlater at 4° C. for 24 hr and then transferred to −80° C. till further processing.

RNA and cDNA generation: mRNA from the tissues collected was isolated with the Dynabcads mRNA DIRECT Purification Kit (Invitrogen) using the Kingfisher (Thermo Fisher Scientific) automated system which isolated mRNA using oligo dT residues bound to the surface of Dynabeads. Either 16 μL or a maximum of 5 μg were used as input for a 40 μL cDNA reaction. cDNA was generated by reverse transcription using the SuperScript IV VILO kit (Thermo Fisher Scientific).

Amplicon generation: Amplicons originating from the reporter gene were amplified via PCR using a set of universal primers for all elements in the library. For each brain tissue samples there were three technical replicates, and for each DRG sample there were four technical replicates starting at the first amplicon PCR step. The cycle number for the amplicon PCR was optimized first with qPCR. Step1 PCR amplifies a region of the 3′UTR of the reporter mRNA under the optimized conditions. DNA obtained from both the AAV library (the one injected into mice) and the AAV genome plasmid library (used to make the AAV library) were used as templates for Step1 PCR as controls in the sequencing run (see below). All technical replicate samples for each biological replicate are pooled together manually in equal molar amounts as the final sequencing library pool.

Amplicon sequencing: Each sample was combined in equal molar amounts into the final sequencing pool. The sequencing pool also included amplicons from the AAV genome plasmid library (used to make the AAV library) and amplicons from the AAV library itself. The molarity of the final library was calculated based on the bioanalyzer trace from a high sensitivity D1000 Tapestation (Agilent 4200) dsDNA in combination with HS dsDNA Qubit fluorometric quantification results. The samples were then pooled at a 60:40 molar ratio with PhiX for diversification of the sample pool. The diversified library pool was further diluted and prepared according to the specification of the Nextseq 2000 P3 200 Cycles Kit (Illumina).

Candidate analysis: A graph showing expression levels in brain vs DRG for the candidate DRG de-targeting library is provided in FIG. 1B. Each dot in the graph represents a single construct sequenced from the library. As seen in FIG. 1B, a number of different constructs achieved decreased DRG expression while maintaining brain expression, or decreased DRG expression to a greater degree than brain expression (see dashed-line boxed region in FIG. 1B). The included negative controls did not significantly affect either brain or DRG expression (light-shaded circles in the solid-line box in FIG. 1B). Construct activity was assessed based on (1) the log₂ fold change (log 2FC) of the construct's expression activity in DRG compared to its abundance in the baseline AAV pool, (2) the log 2FC of the construct's expression activity in hippocampus compared to its abundance in the baseline AAV pool, and (3) the difference between these two log 2FC values. Tissue log 2FC metrics were calculated using DESeq2.

Window scoring for candidate DRG de-targeting endogenous 3′ UTR tiles: As noted above, the selected endogenous 3′ UTR regions were screened as overlapping tiles. Because of the overlap, large variations in activity between adjacent tiles were interpreted to be an indicator of a noisy measurement.

Aggregate “window” activity scores were calculated for each group of 3 adjacent tiles by taking the weighted average of tiles' tissue log 2FC scores, where tiles are weighted by the inverse of the tiles' log 2FC variance. Consequently, tiles that are more well-measured (i.e., less variance in the activity of the 5 constituent barcodes for the construct) were weighted more heavily towards the window average. Window activity scores were used to filter for sequence regions that consistently exhibited a favorable activity profile.

### Example 2

### Selection and Validation of Candidate DRG De-Targeting Elements

Selection of candidate DRG de-targeting elements: Eight (8) elements were selected from the DRG de-targeting screen based on their ability to de-target DRG expression while maintaining brain expression. Six (6) of these candidate DRG de-targeting elements were binding sites for miR-196b-5p, miR-10b-5p, miR-24-2-5p, miR-183-3p, miR-196a-5p, and miR-494-3p. Two (2) of these candidate DRG de-targeting elements were derived from endogenous 3′ UTR sequences, denoted Tile A and Tile B. Table 4 above lists the cognate binding sites shown to have DRG de-targeting activity in the screen (both RNA and DNA), their corresponding full microRNA sequences (RNA), and the sequences of the endogenously-derived DRG de-targeting 3′ UTR Tiles, i.e., Tile A and Tile B (RNA and DNA).

Validation of candidate DRG de-targeting elements: Validation experiments were performed in vivo to assess the DRG de-targeting activity of candidate DRG de-targeting elements that serve as binding sites for miR-196b-5p and miR-183-3p. Separate AAV9 vector preparations were generated (as described above) for each element in which four copies of the miR binding site were added to the 3′ UTR de-targeting region of an EGFP-KASH transgene driven by the EF1α promoter. These constructs also included a WPRE and a human growth hormone (hGH) polyA signal sequence in the 3′ UTR positioned after the de-targeting region. The DNA sequences of the tetrameric miR-196b-5p and miR-183-3p de-targeting regions are provided as SEQ ID NOs: 38 and 39, respectively.

Timed-pregnant female mice (CD-1 IGS wildtype mice from The Jackson Laboratory) were obtained and housed onsite to acclimate to the new environment until they littered. For each AAV vector preparation (miR-196b-5p, miR-183-3p, and a negative control in which the AAV vector contained no de-targeting region in the 3′ UTR), ten (10) post-natal day 1 (P1) pups were injected by stereotaxic intracerebroventricular (sICV) injections. The mice received a total 4.2E10 gc/animal via sICV in a 6 μL dose, 3 μL to each hemisphere, and were treated with Rimadyl the day prior to surgery. Mice were placed on a 12-hour light/12-hour dark cycle, weaned at day 21, and given a standard chow diet ad libitum thereafter. Mice were sacrificed and analyzed at 4 weeks of age.

Mice for each dosed vector were divided between a molecular readout (4 mice) and an immunohistochemistry (IHC) readout (6 mice). For a molecular readout, tissues were collected directly into cold RNAlater (Sigma Aldrich). Tissues included hippocampus, cortex, and all pairs of L1-L6 dorsal root ganglia (DRG). Samples were kept in RNAlater at 4° C. for 24 hrs and then transferred to −80° C. until further processing.

Genomic DNA (gDNA) and total RNA were isolated from the tissue samples using the Qiagen AllPrep DNA/RNA Mini kit. cDNA was generated from the total RNA by reverse transcription using the SuperScript IV VILO kit (Thermo Fisher Scientific). Total cDNA and gDNA concentrations were normalized prior to input into the ddPCR reaction.

For calculation of vector-derived transcript expression (EGFP-KASH), normalized cDNA was used as input for a ddPCR reaction in duplicate. For calculation of AAV vector copy number (VCN), gDNA was used as input for a ddPCR reaction in duplicate. Each reaction was run in two channels to allow each mouse tissue sample (FAM) to be normalized to a housekeeping gene (VIC)—the housekeeping gene used was different for transcript and VCN analysis based on empirically determined best practices. The ddPCR reaction was run using the QX One automated ddPCR instrument under the following conditions: 95° C. for 10 min, 40 cycles of 94° C. at 30 seconds and 60° C. for 1 min, 98° C. for 10 min, and a 4° C. hold for 2 minutes prior to fluorescence readout. Samples with low droplet counts were excluded from analysis. VCN per diploid genome was calculated from the gDNA samples and normalized to the copy number of the reference gene. The transcript copies per microgram of total RNA were calculated from the RNA tissue samples and normalized to the appropriate housekeeping gene. The correlation between VCN and transcript quantification was measured, and data reported as normalized transcript expression per VCN per tissue type for each vector.

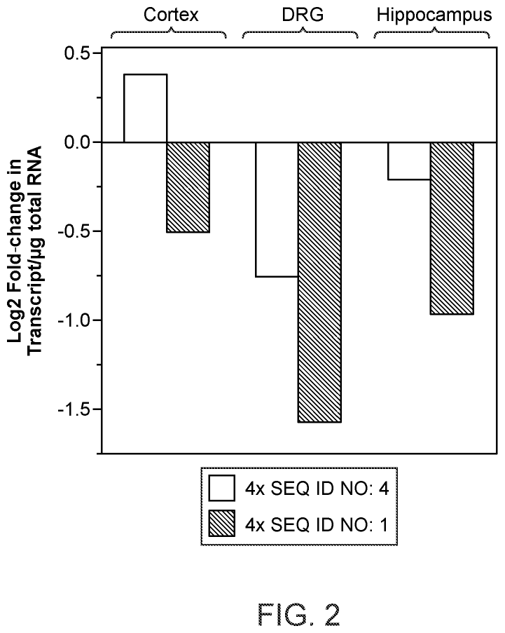

FIG. 2 shows a graph of the log₂ fold-change in EGFP-KASH transcript expression in the cortex, DRG, and hippocampus of mice injected with (1) an AAV9 vector in which the EGFP transcript expressed by the vector includes 4 copies of a binding site for hsa-mir-183-3p (4×SEQ ID NO: 4, provided herein as SEQ ID NO: 44) or (2) an AAV9 vector in which the EGFP transcript expressed by the vector includes 4 copies of a binding site for hsa-mir-196b-5p (4×SEQ ID NO: 1, provided herein as SEQ ID NO: 43) as compared to an AAV9 construct with no de-targeting element. Values used for the calculation are mean EGFP-KASH transcript level per μg of total RNA (normalized to VCN/diploid genome). As seen in this figure, both de-targeting elements reduced EGFP-KASH transcript expression the most in the DRG, with the 4×SEQ ID NO: 4 element reducing expression by ~0.75 fold and the 4×SEQ ID NO: 1 element reducing expression by ~1.5 fold. The 4×SEQ ID NO: 4 element reduced expression by less than ~0.25 fold in the hippocampus while actually increasing expression in the cortex. The 4× SEQ ID NO: 1 element reduced expression in both the cortex and the hippocampus, but to a lesser extent than in the DRG. These results demonstrate that both the hsa-miR-183-3p binding site and the hsa-miR-196b-5p binding site are DRG de-targeting elements.

EGFP-KASH transgene expression at the protein level was assessed in brain and spinal column tissue (which includes the DRG) by immunohistochemistry (IHC). Whole brain and spinal column tissue was collected from mice and fixed in a 4% neutral buffered formalin for 24 hours and then switched to 70% ethanol and kept at 4° C. until processed for paraffin. Slides were prepared from the processed tissues, de-waxed, rehydrated and then heat induced epitope retrieval was performed for 20 min at 95° C. in Citrate pH 6.0 buffer. Slides were mounted in Shandon slide holders, washed with Phosphate Buffered Saline with Tween-20 (PBST) then incubated in antibody dilution buffer (Phosphate Buffered Saline, 1.0% Bovine Serum Albumin, 0.3% Triton-X-100) before incubation with rabbit anti-GFP [Abcam Ab290] in antibody dilution buffer. This was followed by goat anti-rabbit-HRP [ThermoFisher Scientific, A16110] and then Opal 520 [Akoya Biosciences, FP1487001KT].

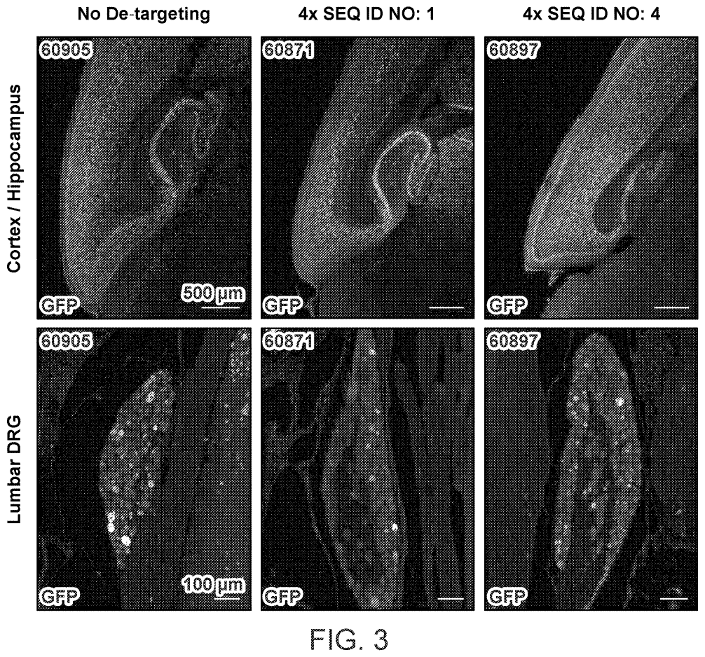

FIG. 3 shows IHC images showing EGFP-KASH expression in cortex/hippocampus (top row) and lumbar DRG (bottom row) tissues harvested from mice injected with the AAV9 vectors in which the EGFP transcript expressed by the vector has (1) no de-targeting elements in the de-targeting region (No De-targeting), (2) tetrameric miR-183-3p binding sites in the de-targeting region (4×SEQ ID NO: 4), or (3) tetrameric miR-196b-5p binding sites in the de-targeting region (4×SEQ ID NO: 1). (Scale of the images is provided in the left-most images.) As seen in FIG. 3, expression of EGFP-KASH was widespread and robust in the cortex/hippocampus of the mice in the three treatment arms. In contrast, DRG expression was more restricted in the 4×SEQ ID NO: 4 and 4×SEQ ID NO: 1 arms than in the No de-targeting arm, demonstrating that these miR binding sites have DRG de-targeting activity.

Validation of candidate DRG de-targeting elements: Additional validation experiments were performed in vivo to assess the DRG de-targeting activity of candidate DRG de-targeting elements in AAV9 vectors. In these vectors, the candidate DRG de-targeting elements were added to the 3′ UTR de-targeting region of an EGFP-KASH transgene driven by the Synapsin 1 (Syn1) promoter. The DRG de-targeting elements tested were as follows (SEQ ID NOs of the RNA sequences in the de-targeting regions of transcript expressed from the AAV9 vectors are indicated): tetrameric binding sites for miR-196b-5p (SEQ ID NO: 43), tetrameric binding sites for miR-183-3p (SEQ ID NO: 44), tetrameric binding sites for miR-24-2-5p (SEQ ID NO: 45), tetrameric binding sites for miR-10b-5p (SEQ ID NO: 46), two copies of a dimer containing binding sites for miR-24-2-5p and miR-10b-5p (SEQ ID NO: 47), two copies of a dimer containing binding sites for miR-196b-5p and miR-10b-5p (SEQ ID NO: 48), Tile A (SEQ ID NO: 7), and Tile B (SEQ ID NO: 9). A control plasmid without a de-targeting region was also generated. The AAV genomic plasmids were generated by Genscript and the AAV9 vectors used in the validation experiments were produced and formulated as previously described herein.

The screen was carried out in neonate post-natal day 1 (P1) C57BL/6 wildtype mice at Charles River Laboratories (CRL) as follows. Pregnant dams were individually housed in cages and maintained on a 12-hour light/12-hour dark cycle with food and water ad libitum. P1 mice (n=5) were injected by stereotaxic intracerebroventricular (sICV) injections and treated with Rimadyl the day prior to surgery. The mice received a total of 1.1E10 gc/animal via sICV injections in a 4 μL dose, 2 μL to each hemisphere. Mice were segregated by sex after weaning and placed 2-5 per cage. Mice were housed in the CRL vivarium for 4 weeks post injection, maintained on a 12-hour light/12-hour dark cycle with food and water ad libitum, and then harvested for IHC analysis of EGFP-KASH protein expression in brain and spinal column tissue (which includes DRG).

Whole brain and spinal column tissue was collected from mice and fixed in a 4% neutral buffered formalin for 24 hours and then switched to 70% ethanol and kept at 4° C. until processed for paraffin. Slides were prepared from the processed tissues, de-waxed, rehydrated and then heat induced epitope retrieval was performed for 20 min at 95° C. in Citrate pH 6.0 buffer. Slides were mounted in Shandon slide holders, washed with Phosphate Buffered Saline with Tween-20 (PBST) then incubated in antibody dilution buffer (Phosphate Buffered Saline, 1.0% Bovine Serum Albumin, 0.3% Triton-X-100) before incubation with rabbit anti-GFP [Abcam Ab290] in antibody dilution buffer. This was followed by goat anti-rabbit-HRP [ThermoFisher A16110] and then Opal 520 [Akoya Biosciences FP1487001KT]. For spinal column tissue, a subsequent heat retrieval step was performed followed by IHC staining cycle with rabbit anti-PGP9.5 [Abcam ab108986] and then with rabbit anti-Histone H3 [Abcam ab1791]. Washes were performed between each step with PBST. Brain and spinal column tissues were counter stained with 4′,6′-diamidino-2-phenylindole, dihydrochloride (DAPI) for and then slides were cover slipped with number 1.5 cover glass.

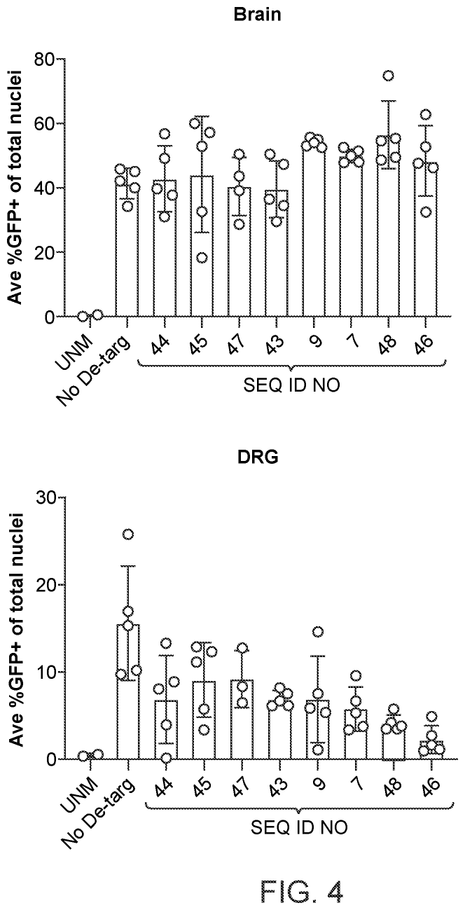

Stained whole slides were scanned at 20× magnification using an Akoya Polaris scanner. Images were imported into an image database (Omero, Glencoe Software) and figures generated using the Figure tool. Image analysis was carried out on full-resolution images (2 pixel/micron) by superimposing two user-defined regions of interest (1.0 mm×1.0 mm) in the somatosensory/motor cortex. In the spinal column, four DRG were identified in each animal. Nuclei segmentation was performed on the DAPI counter stain in the brain samples or the Histone H3 stain in the spinal column tissue using StarDist in ImageJ with a custom model developed in-house. In the spinal column tissue, non-DRG tissue was removed using a mask generated from the PGP9.5 stain, which preferentially stains DRG tissue. Once the nuclei in brain and DRG images were identified, the status of EGFP-KASH staining in these nuclei regions was determined using a trained random forest classifier (Ilastik). The percent EGFP-KASH positive nuclei was calculated by dividing the number of EGFP-KASH positive nuclei by the total number of nuclei in each region of interest. FIG. 4 shows the average and standard deviation (SD) of percent GFP positive nuclei from two cortical regions of the brain tissue (top graph) and the four DRG regions (bottom panel) for each treatment arm (n=5; dots on the graph indicate individual animals). An unmanipulated negative control (UNM) was included (no AAV9 vector injection).

As seen in the top panel of FIG. 4, the percent of cells in the brain expressing EGFP-KASH was not reduced by the inclusion of any of the eight DRG de-targeting elements tested. In fact, certain de-targeting elements had a higher average percentage of EGFP-KASH-expressing cells than controls with no de-targeting element, including SEQ ID NOs: 9, 7, 48 and 46.

In contrast, as shown in the bottom panel of FIG. 4, all eight of the DRG de-targeting elements tested showed a reduction in the percentage of cells in the DRG expressing EGFP-KASH as compared to an AAV9 vector without any DRg de-targeting elements in the de-targeting region. This result demonstrates the DRG de-targeting activity of each of these elements alone and in combination.

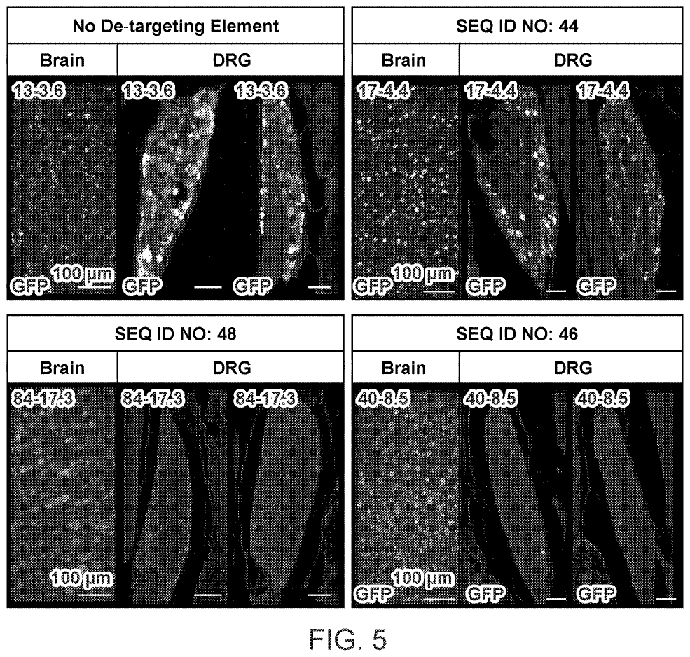

FIG. 5 provides examples of IHC images showing EGFP-KASH expression in brain and DRG from four mice from FIG. 4: the control mouse injected with an AAV9 vector in which the EGFP transcript expressed by the vector had no de-targeting element in the de-targeting region (No De-targeting Element) and three mice which were each injected with a different AAV9 vector in which the EGFP transcript expressed by the vector had the indicated de-targeting element in the de-targeting region (provided as SEQ ID NOs). As is clear from these images, overall expression of EGFP-KASH in the cells of the DRG is reduced significantly as compared to the no de-targeting element control (top left panel) when the DRG de-targeting elements are present, with many cells in the three DRG de-targeting element samples having undetectable levels of EGFP-KASH expression. Brain expression of EGFP-KASH in the same mice is not reduced as compared to the no de-targeting element control (top left panel), demonstrating the tissue specificity of the de-targeting activity of these DRG de-targeting elements.

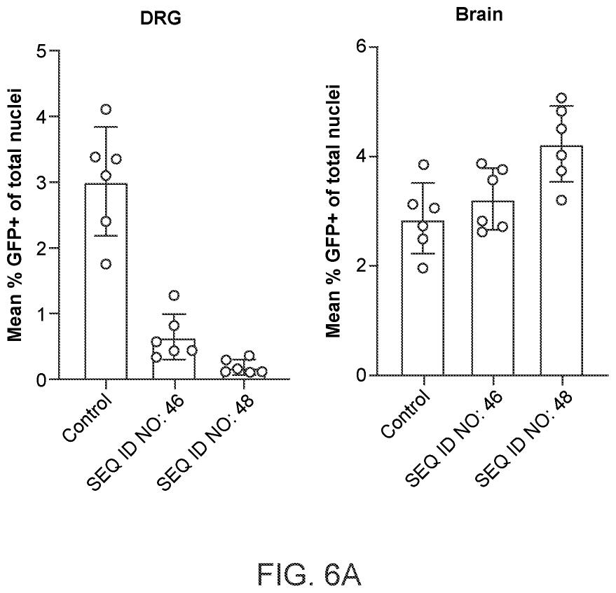

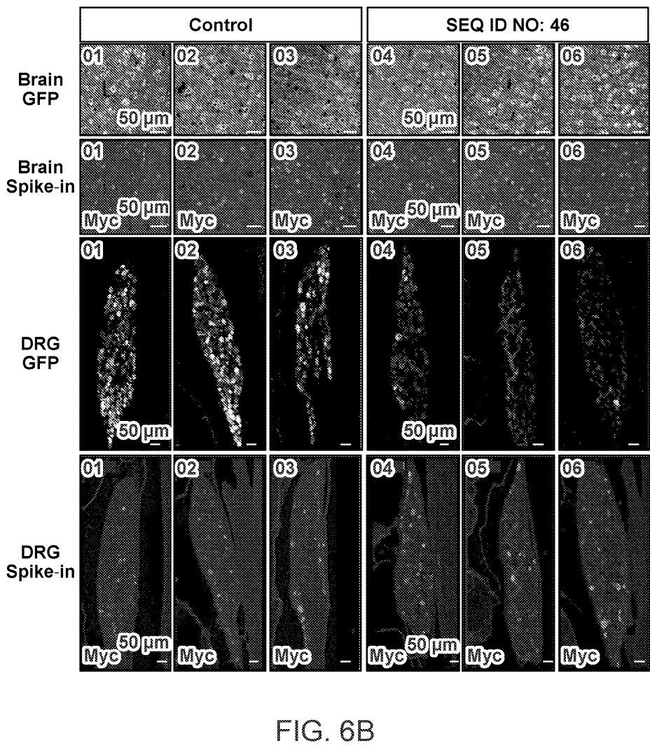

Additional experiments conducted as described above were conducted using specific DRG de-targeting regions. In these experiments, AAV9 with pan-neuronal promoter and control UTR or candidate DRG de-targeting UTRs were administered ICV in mice at P1 (as described above). FIG. 6A shows the % GFP+ nuclei in DRG (left panel) and in brain (right panel). This date clearly demonstrates that inclusion of either a tetrameric binding site for mir-10b-5p (SEQ ID NO: 46) or two copies of a dimer containing binding sites for miR-196b-5p and miR-10b-5p (SEQ ID NO: 48) in the de-targeting region of a transcript reduces transgene expression in DRG but not brain tissue. FIG. 6B shows representative images of mouse samples stained for AAV transgene expression from control vectors with no de-targeting elements in the de-targeting region and vectors in which the de-targeting region of the transcript includes four copies of hsa-mir-10b-5p (SEQ ID NO: 46; same mouse shown in FIG. 6A). Spike-in AAV vector with a myc-tagged mCherry gene under the control of a pan-neuronal promoter was added to controls to assess biodistribution. These experiments show that hsa-mir-10b-5p binding sites in the de-targeting region of an RNA transcript (in this case the 3′UTR) substantially reduce off-target expression in the DRG, while maintaining CNS expression in FIG. 6B (compare GFP expression in Brain with and without the DRG de-targeting element to DRG expression with and without the DRG de-targeting element). These results demonstrate that DRG de-targeting elements show approximately 5 to 16-fold reduction in DRG expression in mice with no reduction brain expression.

### Example 3

### DRG De-Targeting Element Rescues/Reduces Transgene Protein Overexpression in DRG in Mice

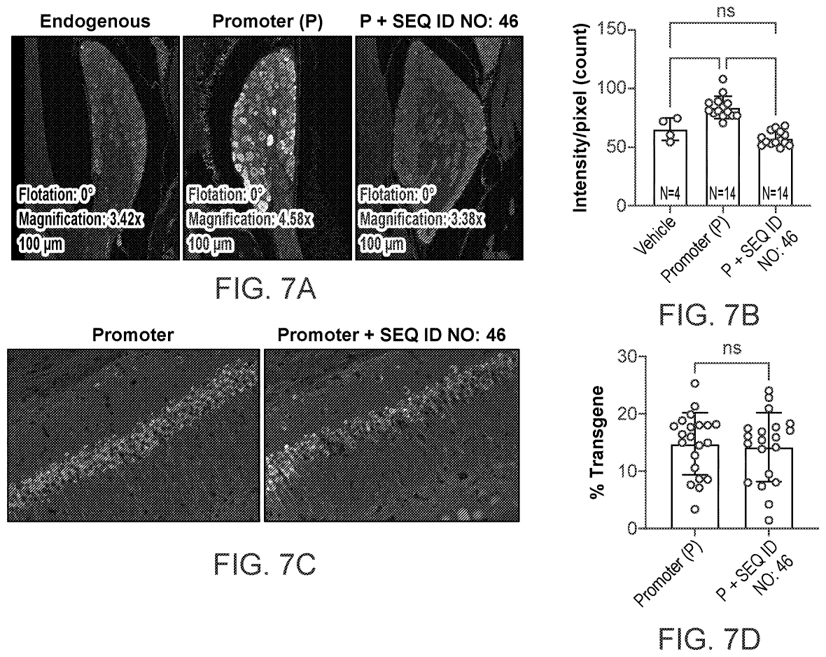

The SEQ ID NO: 46 DRG de-targeting element (which has 4 copies of a binding site for mir-10b-5p) was evaluated in an AAV9 vector with a neuronal transgene under the control of a pan-neuronal promoter. Specifically, the mRNA transcript expressed from this AAV9 vector has SEQ ID NO: 46 in the de-targeting region in the 3′UTR. Mice were injected ICV at P1 (as described above) with (1) vehicle (n=4), (2) an AAV9 vector with a neuronal transgene under the control of a pan-neuronal promoter, where the mRNA transcript expressed does not have a de-targeting element in the de-targeting region in the 3′UTR (n=14), or (3) the DRG de-targetted AAV9 vector described above, i.e., with the neuronal transgene under the control of a pan-neuronal promoter, where the transcript expressed includes a de-targeting region containing SEQ ID NO: 46 (n=14). IHC analysis was performed to detect the expression of the transgene. As seen in the IHC results in FIG. 7A and the quantitation of intensity values in FIG. 7B, the DRG de-targeting element employed (SEQ ID NO: 46) rescues protein overexpression in DRG in mice, i.e., it reduced transgene expression back to the endogenous levels seen with the vehicle injection. This de-targeting element had no effect on brain expression in mouse, as shown in the IHC of FIG. 7C and the intensity values in FIG. 7D.

### Example 4

### DRG De-Targeting Element Rescues/Reduces Transgene Protein Overexpression in DRG in NHP

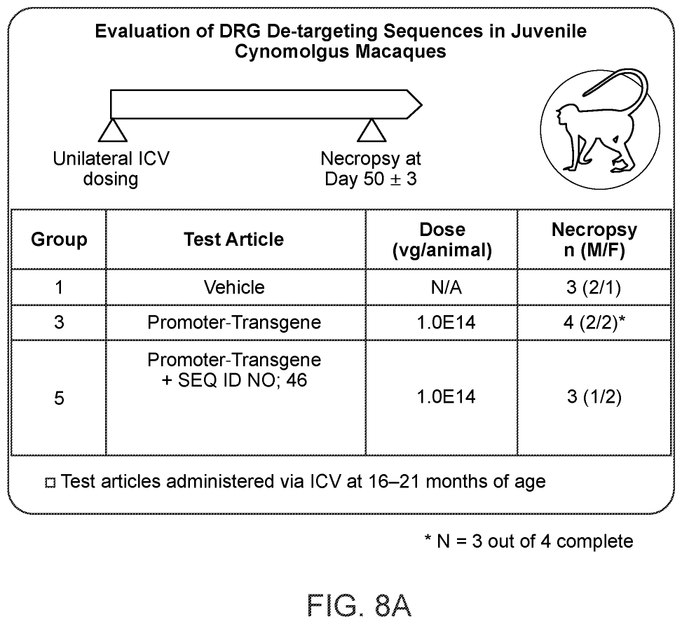

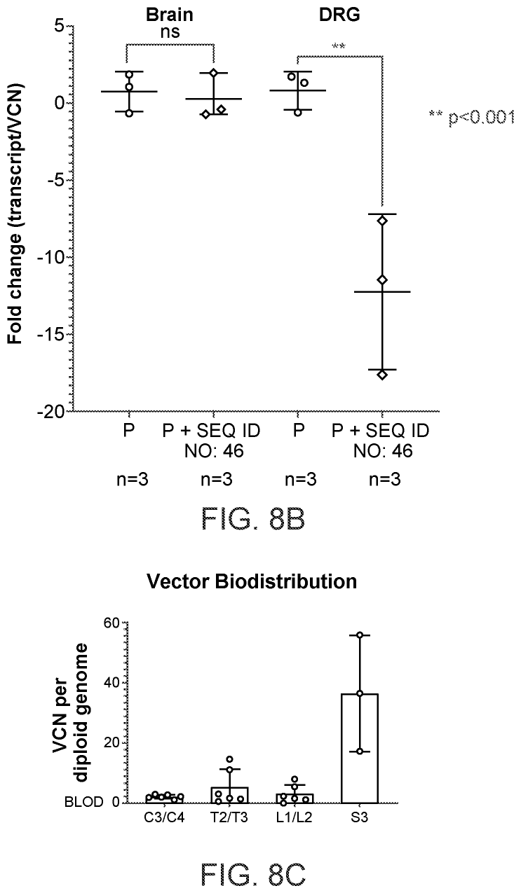

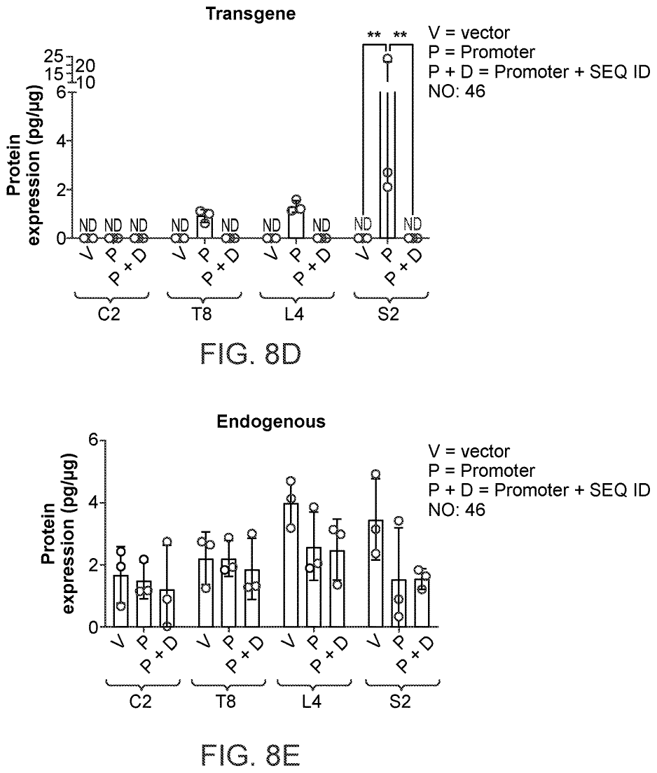

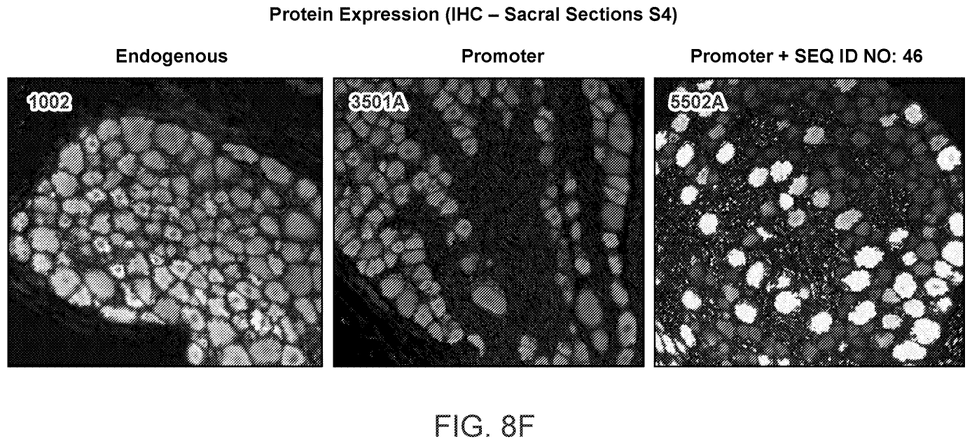

The mouse experiments done in Example 3 were performed in NHP to assess the effect of the DRG de-targeting elements on transgene protein over expression in DRG. FIG. 8A shows experimental design of NHP study to evaluate DRG de-targeting element (SEQ ID NO: 46) in NHP. NHP were injected ICV (as described above) with vehicle (Group 1), an AAV with a pan neuronal promoter driving the expression of an mRNA transcript encoding the neuronal gene (Group 3), or AAV with a pan neuronal promoter driving the expression of an mRNA transcript encoding the neuronal gene that also includes SEQ ID NO: 46 in the de-targeting region of the 3′UTR (Group 5). FIG. 8B shows the molecular analysis of AAV transcript expression between Group 3 (Promoter, denoted “P” in the figure) and Group 5 (P+SEQ ID NO: 46). AAV-driven transcript expression in brain and DRG sections was evaluated by RT-ddPCR analysis using vector-specific primer/probes. Total RNA expression was normalized to AAV genome copy number in each section and represented as a fold change to the promoter only condition. No difference in AAV-driven transgene expression in forebrain and midbrain was detected while a >10-fold reduction in AAV-mediated transcript expression was seen across DRG sections (cervical, thoracic, lumbar, and sacral were analyzed). FIG. 8C shows vector biodistribution in cervical (C), thoracic (T), lumbar (L), and sacral(S) spinal cord DRG segments. Expression in DRG is found primarily in the sacral DRG tissue. Protein expression driven by the transgene (FIG. 8D) and endogenously (FIG. 8E) by MSD-ELISA (using reagents specific for either the transgene-encoded protein or the endogenous protein) is shown in cervical (C), thoracic (T), lumbar (L), and sacral(S) spinal cord DRG segments. This figure shows that the presence of DRG de-targeting element SEQ ID NO: 46 in the de-targeting region of an mRNA transcript reduces expression of the transgene encoded protein but does not significantly impact expression of the endogenous gene. IHC staining for both the transgene encoded and endogenous protein in sacral(S) DRG section is show in FIG. 8F. This figure shows that without the DRG de-targeting element, protein expression was significantly higher in the sacral DRG tissue (center panel) as compared to the endogenous levels (left panel). Inclusion of binding sites for mir-10b-5p, a DRG de-targeting element, reduced the level of the protein expression to endogenous levels (right panel).

### Example 5

### Liver De-Targeting Element Identification

Element selection for library generation: Elements were initially harvested from annotated 3′ untranslated regions (3′ UTR) of genomic sequences curated in the AURA database (Atlas of UTR regulatory activity; Dassi E, Re A, Leo S, Tebaldi T, Pasini L, Peroni D and Quattrone A. (2014) AURA 2: Empowering discovery of post-transcriptional networks. Translation, 2(1): e27738.), and based on proximity to liver-depleted genes according to expression data from the Genotype-Tissue Expression portal. The genes were screened for high expression in at least one non liver tissue. 100 genes were selected based on depleted expression in the liver but high expression in at least one other tissue. The genomic sequence of the 3′ UTR associated with each of these 100 genes was segmented into overlapping 127 base pair (bp) candidate elements termed “tiles”, using a sliding window distance of 25 bp.

Further elements were harvested from the Functional Annotation of the Mammalian Genome database (Fantom5, Lizio M, et al. Gateways to the FANTOM5 promoter level mammalian expression atlas. Genome Biol 16:22 (2015). 10.1186/s13059-014-0560-6), the mircoRNA Expression and Sequence Analysis database (mESA, Koray D. Kaya, Gökhan Karakülah, Cengiz M. Yakιcιer, Aybar C. Acar, Özlen Konu, mESAdb: microRNA Expression and Sequence Analysis Database, Nucleic Acids Research, Volume 39, Issue suppl_1, 1 Jan. 2011, Pages D170-D180,) and Minatel et al (Minatel B C, Martinez V D, Ng K W, et al. Large-scale discovery of previously undetected microRNAs specific to human liver. Hum Genomics. 2018; 12 (1): 16.). Each miRNA selected was based on a) maintaining a minimal liver expression, b) having a maximal expression in at least one other tissue type, and c) having a maximal expression in all other tissues. The selection of a miRNA was based on whether its expression in the liver was significantly different from all other tissues based on Grubb's Statistic. Expression was manually checked to ensure that the differential expression of the miRNA was higher relative to other tissues. All miRNA for consideration from the above databases were compiled and checked for redundancy between names of the miRNA.

Control element pool: The control element pool was composed of either published miRNA response elements or previously identified elements. These controls served as benchmarks and diagnostic reference points due to their predictable expression characteristics. The control elements roughly fall into three categories that include a) miRNA or 3′UTR with known expression profiles, b) various promoters with known expression profiles, and c) random sequences in the promoter position or in the 3′UTR. The miRNA, 3′UTR, and random sequence controls were driven by the same promoter used in the screen. The promoter controls contained no element or sequence in their 3′UTR region besides those needed for amplicon generation and molecular barcode identifier. Each element in the control pool was assigned a barcode, located in the 3′ UTR of the gene of interest.

Library composition: binding sites for the selected miRNAs were represented as a tetramer repeat with an 8 bp spacer in between the given miRNA binding site and comprised 2,000 unique elements. Those miRNA that had supporting evidence in the literature or were obtained from the FANTOM5 database were constructed into de-targeting regions that contained 1, 2, 3, or 4 copies of the given miRNA binding site and comprised ~350 unique elements.

Unique barcodes assigned to library elements for oligo pool synthesis: To prepare the designed library of elements for oligo pool synthesis, each candidate element's sequence was checked for the presence of restriction enzyme sites necessary for downstream cloning, and those containing these sequences were discarded. Those elements whose sequence contained the recognition sites for BsrGI, EcoRI, XbaI, AscI, and KpnI were removed from the library pool. Each of the remaining elements were assigned a barcode element. Each de-targeting element and its barcode was concatenated with flanking sequences for cloning into the vector backbone. This library was synthesized as single-stranded DNA by Twist Biosciences.

Plasmid library synthesis: The experimental plasmid library was constructed from single stranded oligo pools and ligated into a common plasmid backbone. The single stranded oligos were amplified via PCR using a common set of primers and further ligated into the 3′UTR of a transgene in the screening plasmid. The plasmid is composed of the following: (minCMV promoter)-(nano Luciferase)-(MCS)-(Liver de-targeting element)-(Amplicon barcode and primer sites)-(hGH polyA). The library was transformed into electrocompetent E. coli cells and was both plated onto an agar plate for Sanger Sequencing as well into a 200 mL LB liquid culture. The agar plate colonies were Sanger sequenced to validate that the elements were ligated correctly and there were no indels. The library was then isolated from the 200 mL culture using the Zymogen Maxiprep kit (Zymogen). This product was then pooled with the control spike-in plasmid library (See Control element pool section) to create the final library used for vector production.

AAV Vector preparation: All vectors were produced in adherent HEK293T cells in DMEM+10% FBS. Cells were transfected using PEI-MAX with the library of elements and helper plasmids, which include the cis ITR-containing plasmid, the trans plasmid pAAVX encoding AAV2 replication, and AAVX capsid genes and pALD-X80 the adenoviral helper plasmid. AAV was harvested from the cells and purified from the lysate using an ultra-centrifugation gradient of Iodixanol and further polished with an anion exchange column, followed by concentration and formulation in PBS with 0.001% pluronic.

Animals: The screen was carried out in adult female C57BL/6J mice (The Jackson Laboratory). Mice were obtained as 6- to 8-week-old adults and housed onsite for 3 days to acclimate to the new environment. Animals were given a standard chow diet ad libitum and on a 12 light/12dark light cycle. After injection, the mice were housed individually in a vivarium until they were sacrificed.

AAV injection: Mice (n=5) were injected by intravenous (IV) tail-vein injection and stereotaxic direct hippocampal injections. Animals were treated with Rimadyl the day prior to surgery. The mice received 3E12 genome copies (gc)/animal via IV in a single dose and a total of 1.8E11 gc/animal spread across four 1.5 μL injections into the left, right, dorsal, and ventral regions of the hippocampus. Animals were incubated with the virus in the vivarium for 3 weeks post injection.

Sample collection: Tissues, including hippocampus and liver, were collected directly into RNAlater (Sigma Aldrich). Samples were kept in RNAlater at 4° C. for 24 hr and then transferred to −80° C. till further processing.

RNA and cDNA generation: RNA from the samples was isolated with the RNeasy Mini column method using the standard protocol (Qiagen), which isolated total RNA. Total RNA concentrations were normalized prior to input into the cDNA reaction. cDNA was generated by reverse transcription using the SuperScript IV VILO kit using oligo dT primers (Thermo Fisher Scientific).

Amplicon generation: Amplicons originating from the reporter gene were amplified via PCR using a set of universal primers for all elements in the library. For each sample there were four technical replicates starting at the first amplicon PCR step. The cycle number for the amplicon PCR was optimized first with qPCR. Step1 PCR amplifies a region of the 3′UTR of the reporter mRNA under the optimized conditions. Briefly, AAV is digested with DNasel (New England Biolabs) to remove the capsid and then used directly for Step1 PCR as above. All technical replicate samples for each biological replicate are pooled together in equal molar amounts as the final sequencing library pool.

Amplicon sequencing: Each sample was combined in equal molar amounts into the final sequencing pool, which included each biological replicates' PCR samples, the amplicons from the plasmid pool used to make the AAV, and the amplicons from the dosed AAV library. The samples were then pooled at a 60:40 molar ratio with PhiX for diversification of the sample pool. The diversified library pool was further diluted and prepared according to the specification of the Nextseq 500 High Kit (Illumina).

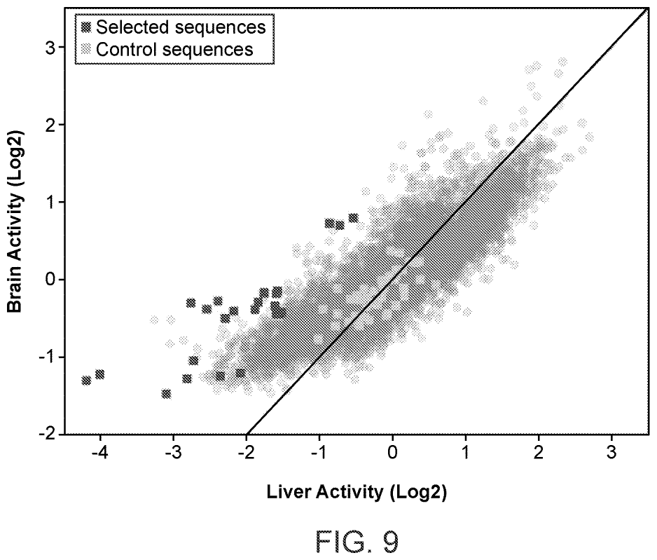

Candidate selection: A scatter plot showing brain activity versus liver activity for the tested constructs is provided in FIG. 9. As seen in FIG. 9, many different constructs achieved decreased liver expression while maintaining brain expression, or decreased liver expression to a greater degree than brain expression (dark shaded boxes). The included negative controls (light shaded boxes) did not affect either brain or liver expression. Construct activity was assessed based on (1) the log 2 fold change (log 2FC) of the construct's expression activity in liver compared to its abundance in the baseline AAV pool, (2) the log 2FC of the construct's expression activity in hippocampus compared to its abundance in the baseline AAV pool, and (3) the difference between these two log 2FC values. Tissue log 2FC metrics were calculated using DESeq2 (see below for more details). The log 2 fold changes in brain and liver expression of selected constructs are also provided in Table 5. The SEQ ID NOs provided in Table 5 represent the RNA sequence present in the 3′ UTR of the mRNA transcript expressed by the library vector. The elements listed in Table 5 (apart from the negative control sequence) show greater reductions in liver expression than in brain expression, indicating that these elements can de-target liver expression. As shown in Table 5, elements provided herein de-target liver expression both when used singly, and when provided as tandem repeats of 2×, 3× or 4×.

TABLE 5

| | Log2 fold | Log2 fold |
|---|---|---|
| Description | change in liver | change in brain |
| SEQ ID NO: 57, 1 copy | −1.091138 | −0.502499 |
| SEQ ID NO: 57, 2 copies | −2.034107 | −1.074236 |
| SEQ ID NO: 57, 3 copies | −2.008143 | −1.313751 |
| SEQ ID NO: 57, 4 copies | −1.58071 | −1.004799 |
| SEQ ID NO: 59, 1 copy | −0.149644 | −0.115088 |
| SEQ ID NO: 59, 2 copies | −0.620037 | −0.477955 |
| SEQ ID NO: 59, 3 copies | −0.649259 | −0.352649 |
| SEQ ID NO: 59, 4 copies | −1.204078 | −0.907602 |
| SEQ ID NO: 60, 4 copies | −1.886355 | −0.388039 |
| SEQ ID NO: 61, 4 copies | −1.602891 | −0.404599 |
| SEQ ID NO: 62, 1 copy | −1.191418 | −0.151467 |
| SEQ ID NO: 62, 2 copies | −1.55778 | −0.738756 |
| SEQ ID NO: 62, 3 copies | −0.296954 | −0.119777 |
| SEQ ID NO: 63, 1 copy | −2.151987 | −0.492311 |
| SEQ ID NO: 63, 2 copies | −1.815708 | −0.787522 |
| SEQ ID NO: 63, 3 copies | −0.766551 | −0.469931 |
| SEQ ID NO: 63, 4 copies | −0.986991 | −0.333258 |
| SEQ ID NO: 113 | −2.813155 | −1.276399 |
| (positive control) | | |
| SEQ ID NO: 41 | 0.029446 | −0.000071 |
| (negative control) | | |

Another de-targeting element, SEQ ID NO: 64, was identified in a similarly conducted screen. As shown in Table 6, SEQ ID NO: 64 also de-targeted liver expression both when present in the 3′ UTR of an mRNA transcript as a single copy and when provided as a tandem repeat of 2× or 4×.

TABLE 6

| | Log2 fold | Log2 fold |
|---|---|---|
| Description | change in liver | change in brain |
| SEQ ID NO: 64, 1 copy | −1.8481975 | −0.4314376 |
| SEQ ID NO: 64, 2 copies | −2.1350587 | −0.3656623 |
| SEQ ID NO: 64, 4 copies | −2.0649475 | 0.03460468 |

Window scoring for endogenous 3′ UTR tiles: ~7,000 constructs in the de-targeting screen were sequence elements derived from the 3′ UTR regions of endogenous genes that displayed favorable expression patterns (see “Selection of endogenous 3′ UTR-derived elements” section for more details). Each endogenous 3′ UTR region is spanned by overlapping 127 bp sequence “tiles” that are each staggered by 25 bp from the previous tile. Since neighboring 3′ UTR tiles share ~80% of their sequence such tiles are expected to have similar activity profiles in general; large variations in activity between adjacent tiles may be an indicator of noisy measurement.

Aggregate “window” activity scores were calculated for each group of 3 adjacent tiles by taking the weighted average of tiles' tissue log 2FC scores, where tiles are weighted by the inverse of the tiles' log 2FC variance. Consequently, tiles that are more well-measured (i.e., less variance in the activity of the 5 constituent barcodes for the construct) were weighted more heavily towards the window average. Window activity scores were used to filter for sequence regions that consistently exhibited a favorable activity profile.

Selecting elements for validation: Several sequences were selected based on lower liver expression and preserved expression in hippocampus to further validate via IHC and ELISA.

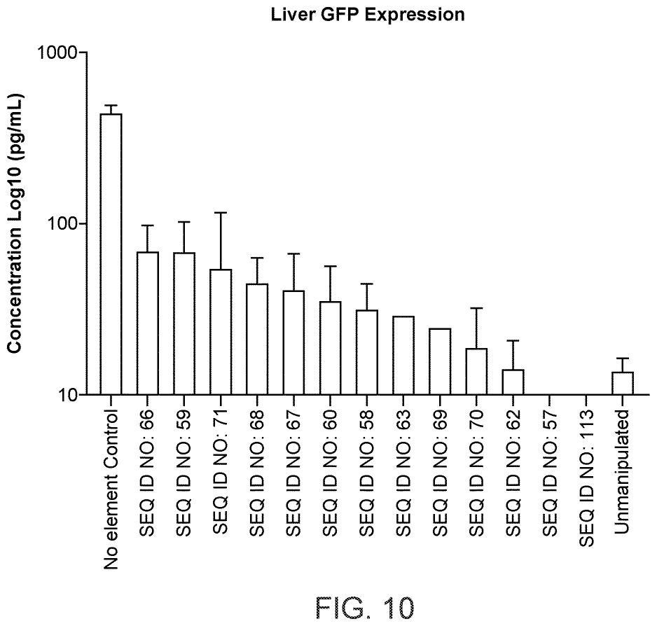

ELISA: Samples for protein analysis were from adult female C57BL/6J mice. Each mouse was dosed with 5.0E11 vg/mouse of AAV9 via intravenous injection (tail vein) for a 3-week incubation period. The vector was created as stated in AAV Vector Preparation. The GOI of the vector was as followed: (AAV2 ITR)-(EF1α(short))-mCherry-KASH-spA-(CTCF insulator)-(CMV promoter)-EGFP-KASH-(Liver De-targeting Element)-sPA-(AAV2 ITR). The Liver De-targeting Elements were selected as described above and are listed as a test sequences in Table 4 above. Both the left lateral lobe of the liver and one hemisphere of the cortex, excluding the olfactory bulb, cerebellum, and other hindbrain tissues were collected from PBS perfused mice. Samples were homogenized in Buffer PRT (Abcam, ab171581 and ab221829) with 2.8 mm ceramic beads (OMNI Intl.) in a mechanical homogenizer for 1 min at 4° C. and clarified by centrifugation. Total protein was quantified using Micro BCA (ThermoFisher) and normalized in Buffer PRT. EGFP and mCherry proteins were quantified separately using ELISA (Abcam) and quantified based on a standard curve generated from a recombinant protein for each respective assay. As seen in FIG. 10, the majority of the de-targeting elements tested showed a large decrease in liver expression compared to the control.

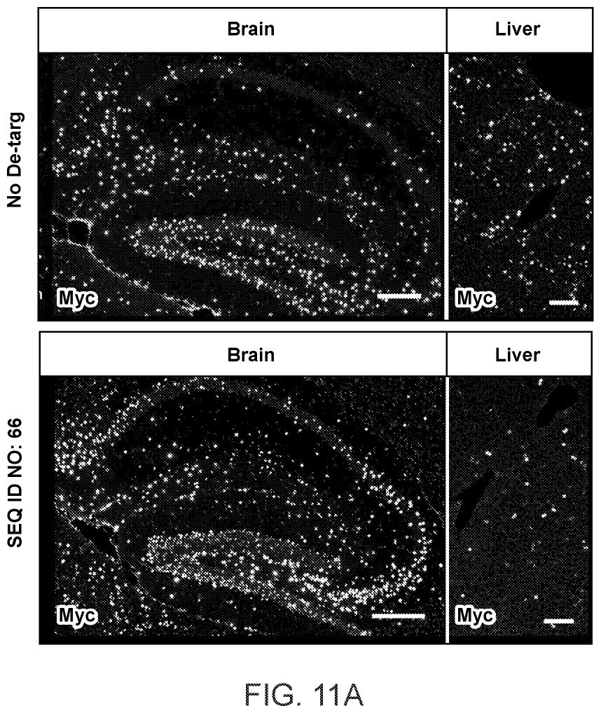

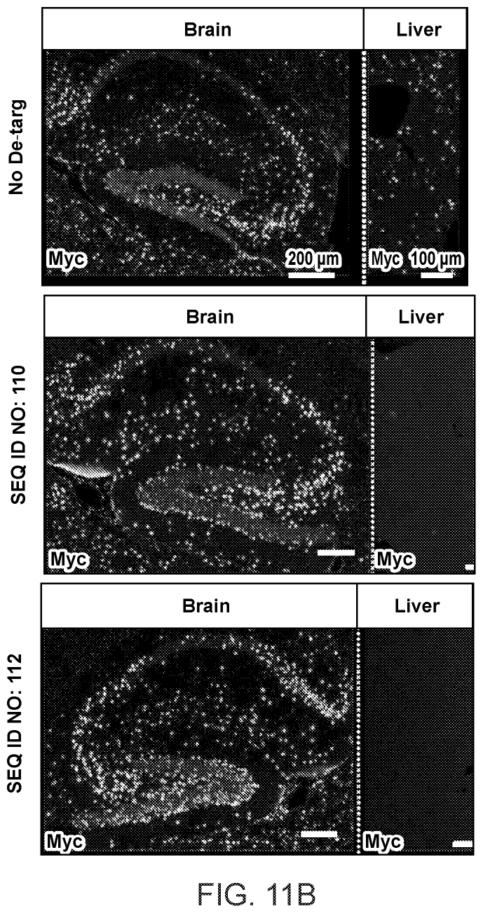

Tissue preparation and Immunohistochemical (IHC) staining: Following saline perfusion whole brain and liver tissue was collected and fixed in 4% neutral buffered formalin for 24 hrs then switched to 70% ETOH and kept at 4° C. until processing. Tissue was processed for formalin fixation and paraffin embedding by an external provider. Following parasagittal embedding of the brain, 5 μm sections were cut onto glass slides. Two transverse sections of the liver lobe were collected on one slide for each animal. For IHC slides were de-waxed, rehydrated and then heat induced epitope retrieval was performed for 20 min at 95° C. in Citrate pH6 buffer. Slides were mounted in Shandon slide holders, washed with Phosphate Buffered Saline with Tween-20 (PBST) then incubated for 15 in antibody dilution buffer (Phosphate Buffered Saline, 1.0% Bovine Serum Albumin, 0.3% Triton-X-100) before incubating overnight at 4° C. with a 1:25,000 dilution of rabbit anti-Myc [Abcam ab9106] in antibody dilution buffer. This was followed by goat anti-rabbit-HRP [Thermo A16110] for 1 hour then Opal 520 [Akoya Biosciences FP1487001KT] for 10 min. Washes were performed between each step with PBST. Nuclei were stained with 4′,6′-diamidino-2-phenylindole, dihydrochloride (DAPI) for 15 min then cover-slipped with #1.5 cover glass. FIG. 11A shows representative images of brain and liver expression from a mouse treated with a control vector without a de-targeting element (“No De-targ”) and brain and liver expression from a mouse treated with a vector encoding an RNA containing SEQ ID NO: 66 in the 3′ UTR (FIG. 11A, bottom panel). This experiment shows the liver-de-targeting activity of SEQ ID NO: 66.

Similar IHC experiments were performed with mice injected intracisternal magna (ICM) with the following AAV vectors: (i) a control vector that expresses a transcript (encoding the myc-tagged protein), under the control of a pan-neuronal promoter, without a de-targeting element (top panel; No De-targ), (ii) a vector that expresses a transcript (encoding the myc-tagged protein), under the control of a pan-neuronal promoter, containing SEQ ID NO: 110 in its de-targeting region (middle panels; SEQ ID NO: 110 includes 2 copies of the hsa-mir-19a-3p binding site (SEQ ID NO: 65), 2 copies of the hsa-mir-1258-5p binding site (SEQ ID NO: 58), and 2 copies of the hsa-mir-17-5p binding site (SEQ ID NO: 60)), and (iii) a vector that expresses a transcript (encoding the myc-tagged protein), under the control of a pan-neuronal promoter, containing SEQ ID NO: 112 in its de-targeting region (lower panels; SEQ ID NO: 112 includes 3 copies of the hsa-mir-122-3p binding site (SEQ ID NO: 62)). (1), SEQ ID NO: 110 in the 3′ UTR (FIG. 11B, middle panel), or SEQ ID NO: 112 in the 3′ UTR (FIG. 11B, bottom panel). As seen in FIGS. 11A and 11B, these three elements, when present in an RNA transcript, have liver de-targeting activity.

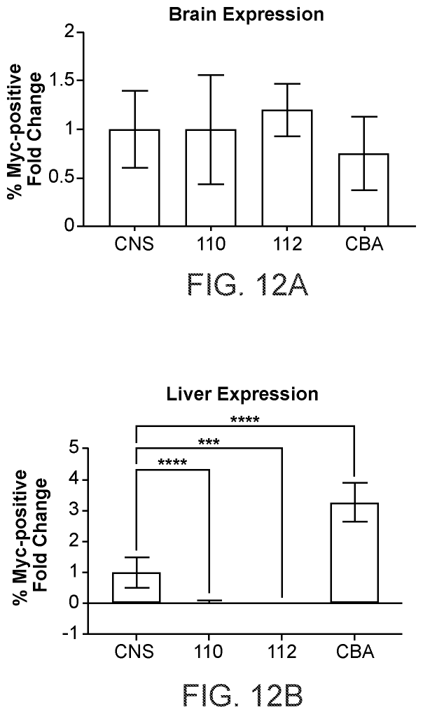

FIGS. 12A and 12B show the fold-change in myc-positive cells present in brain tissue (FIG. 12A) and liver tissue (FIG. 12B) in the mice from FIG. 11B as well as a fourth mouse injected ICM with an AAV vector that expresses the myc-tagged protein under the control of a ubiquitous chicken beta actin promoter (“CBA”) and without a de-targeting element. This data demonstrates the liver de-targeting activity of SEQ ID NO: 110 and SEQ ID NO: 112 (*** and **** indicate significant differences in expression).

### Example 6

### In Vitro Validation of Selected Liver De-Targeting Elements

Induced pluripotent stem cells (IPSCs) were used to validate the identified liver de-targeting elements in human cells. Adeno-associated virus (AAV) particles were prepared using the AAVDJ serotype. Each viral preparation comprised a genome which included an EF1α promoter, a coding sequence for an enhanced green fluorescent protein fused to a KASH domain (eGFP-KASH) and either a random sequence (SEQ ID NO: 55) or a liver de-targeting element (SEQ ID NO: 68, 60, or 62).

IPSC derived glutamatergic neurons and hepatocytes were plated in 24 well plates. After 48 hrs the IPSC derived cells were transduced with the different AAV constructs at a multiplicity of infection (MOI) of 5×10\hat{ }5. The cells were then incubated for an additional 72 hrs before being harvested for RNA and DNA extraction. Quantitative polymerase chain reaction was used to assay levels of eGFP-KASH mRNA, compared to an internal control (GAPDH). The results of the in vitro validation are shown in Table 7.

TABLE 7
In vitro validation

| | Hepatocyte | Glutamatergic |
|---|---|---|
| Element | expression | neuron expression |
| SEQ ID NO: 55 | 1 | 1 |
| 2x SEQ ID NO: 68 | 1.41 | 1.15 |
| 4x SEQ ID NO: 60 | 0.26 | 0.52 |
| 4x SEQ ID NO: 62 | 0.19 | 1.06 |

### Example 6: Liver De-Targeting in Non-Human Primates

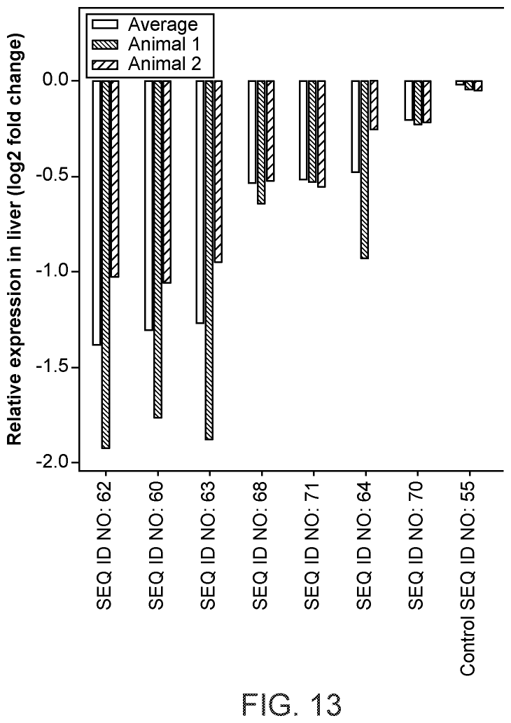

To assess conservation between mouse and non-human primate (NHP) several de-targeting elements were selected for an NHP study. AAVs with different de-targeting elements were prepared as described above and pooled for administration by unilateral intracerebroventricular (ICV) injection at a dose of 10\hat{ }14 viral genomes/animal into juvenile cynomolgus macaques between 16-21 months of age (n=2). Animals were necropsied at approximately 50 days after treatment, and liver and brain tissue were harvested for DNA and RNA extraction. AAV-driven transcript expression was evaluated by reverse transcription droplet digital PCR analysis using vector-specific primer/probes. Total RNA expression was normalized to AAV genome copy number in each sample. FIG. 4 shows relative expression in liver (log₂ of fold change) for several different liver deetargeting elements in NHPs in each of the treated animals, and averaged. As shown in FIG. 13, all the tested elements showed decreased expression in the liver compared to the control sequence, with SEQ ID NOs: 60, 62, and 63 showing the most significant reductions in liver expression.

### Example 7

### Combinatorial DRG and Liver De-Targeting Element Assay in Mice

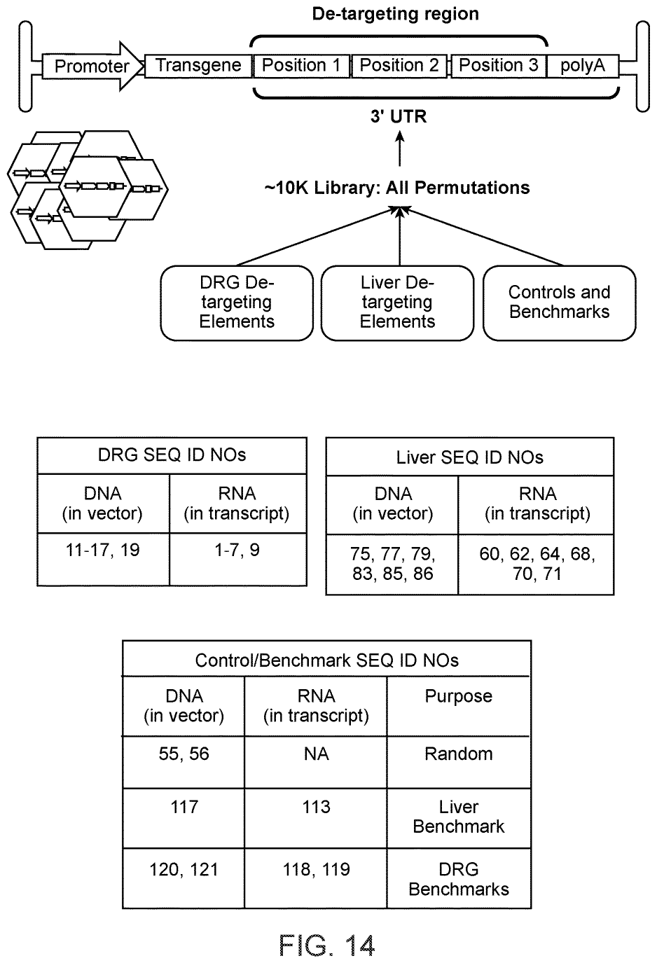

DRG and Liver de-targeting activity of select de-targeting elements were assessed combinatorially in mice. FIG. 14A shows schematic of the design and construction of combinatorial libraries using select DRG and liver de-targeting candidates of the present disclosure. De-targeting elements included in this combinatorial library are shown in the Tables in FIG. 14, which provides both the DNA sequence that would be included in position 1, 2, and/or 3 of the de-targeting region in the AAV vector construct and the corresponding RNA sequence as it would appear in the 3′UTR of an RNA transcript expressed from the AAV vector in a vivo. As noted in the schematic in FIG. 14, all possible permutations of all elements (i.e., DRG de-targeting, liver de-targeting, and control/benchmark) are included in the combinatorial library. The library was administered to mice by unilateral intracerebroventricular (ICV) injection as described above. DRG, liver, and brain tissue was harvested for DNA and RNA extraction. AAV-driven transcript expression (EGFP-KASH) was evaluated by reverse transcription droplet digital PCR analysis using vector-specific primer/probes. Total RNA expression was normalized to AAV genome copy number in each sample. RNA and DNA sequence information was used to determine the ratio of vector genome copy number to transcript expression level as well as identify the de-targeting elements present for each detected RNA and DNA species.

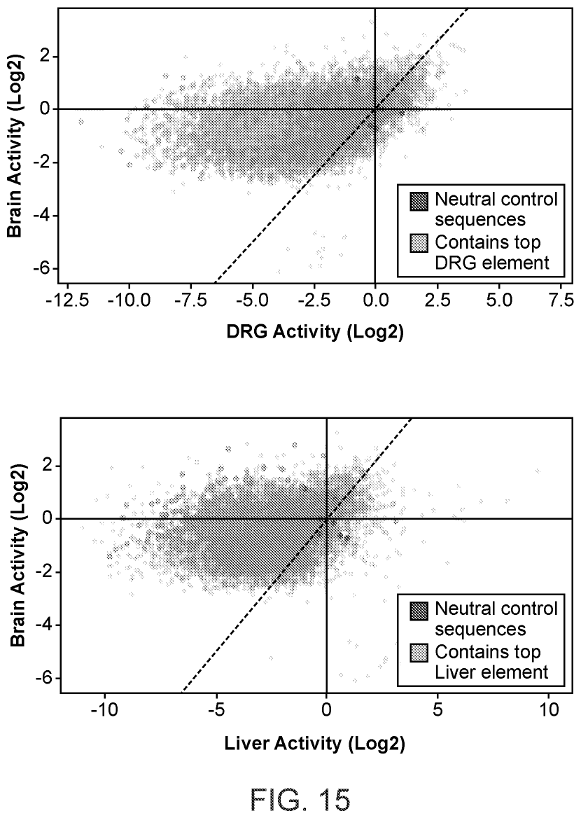

FIG. 15 shows scatter plots of the expression pattern of individual vectors in the AAV library in mouse tissues. The top panel compares the log₂ brain expression activity (y axis) versus the log₂ DRG expression activity (x axis) and the bottom panel the log₂ brain expression activity (y axis) versus the log₂ liver expression activity (x axis). In the top panel, the highlighted vector points include one or more of the top DRG de-targeting elements. In the bottom panel, the highlighted vector points include one or more of the top liver de-targeting elements. In both panels, the dark vector points are those with only neutral control sequences. This combinatorial screen data demonstrates strong de-targeting skew and range for DRG and liver (x axes) de-targeting elements and that the effect of these de-targeting elements was limited to the desired tissue, as there was little to no effect in brain transgene expression (data clustering about 0 of the y axes). The large dynamic range provides various options for the generation of highly specified de-targeting constructs.

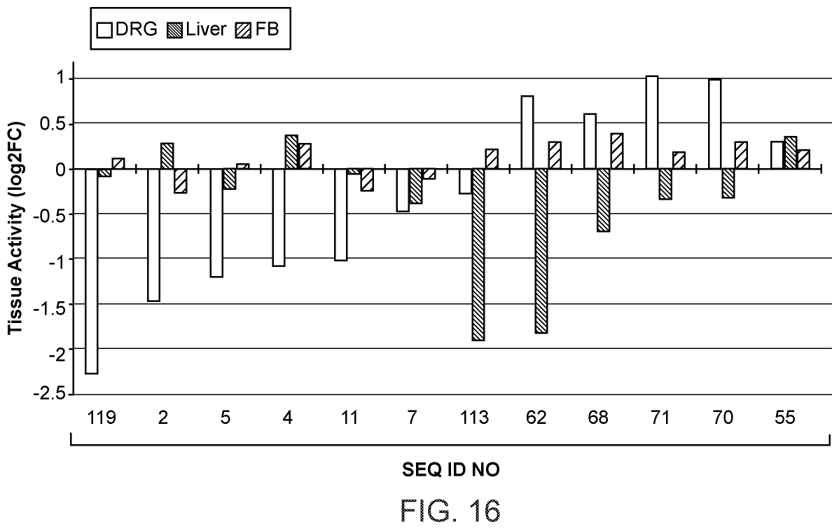

FIG. 16 shows the effect of individual elements on transgene expression in DRG, liver, and Forebrain tissue in mice injected with the combinatorial library (shown as log 2 fold change). These values were determined by recovering the transcribed library from DRG, liver, and brain tissue sections using amplicon sequencing of RNA/AAV DNA and NGS quantification for differential expression. Regression-based models of tissue expression showed top contributing elements across thousands of data points/instances. The data is presented as log 2 fold-change in the specified tissue in the presence and absence of the de-targeting element identified on the x axis by SEQ ID NO. Each SEQ ID NO represents the RNA sequence present in the transcript in the de-targeting region of the expressed transcript except for SEQ ID NO: 55, which is the DNA sequence of the random control element in the AAV vector library. SEQ ID NO: 119 is the DRG de-targeting benchmark and SEQ ID NO: 113 is the liver de-targeting benchmark. Modelling results identified top 3′UTR sequence elements with selective DRG de-targeting while maintaining expression in brain. As seen in FIG. 16, SEQ ID NOs: 2, 5, 4, and 11 have DRG de-targeting activity, SEQ ID NOs: 62, 68, 71, and 70 have liver de-targeting activity, and SEQ ID NO: 7 has de-targeting activity in both tissues.

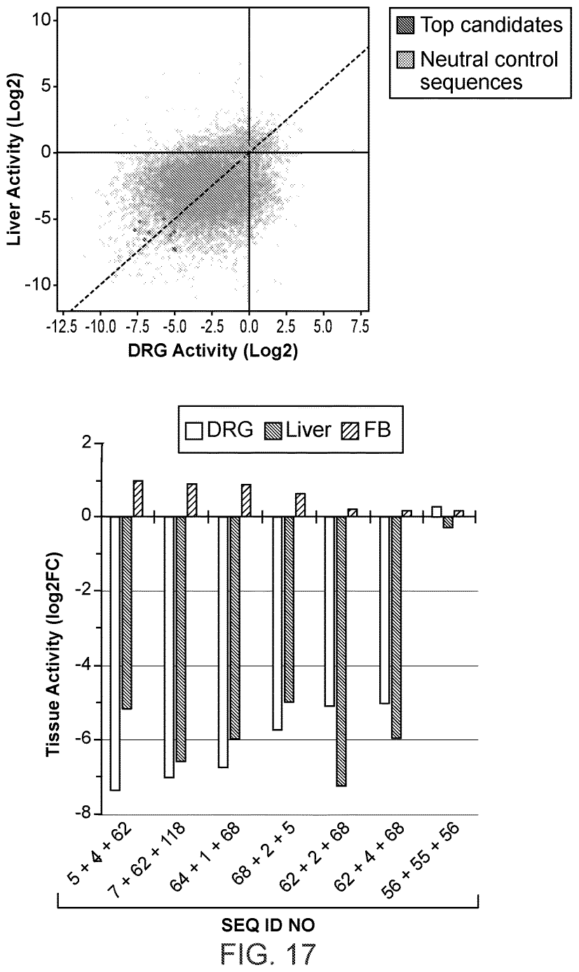

The combinatorial data was re-analyzed to identify individual constructs that de-target both DRG and liver tissues (FIG. 17). As shown in the top panel of FIG. 17, Candidate combinatorial de-targeting regions were identified that exhibit strong de-targeting in both DRG and liver (dark highlighted circles in the scatter plot). These candidate combinatorial de-targeting regions include at least one DRG and at least one liver de-targeting element in the de-targeting region of the 3′UTR. Select combinatorial de-targeting elements highlighted in the scatter plot were further analyzed and shown to have no negative effect on transgene expression in brain tissue (FIG. 17, bottom graph). This graph shows the log₂ fold-change in the specified tissue in the presence and absence of the combinatorial de-targeting element identified on the x axis by the three SEQ ID NOs. The three SEQ ID NOs represents the RNA sequences present in the transcript in the de-targeting region of the expressed transcript, except for SEQ ID NOS: 55 and 56 in the last column, which are the DNA sequences of the random control elements in the AAV vector library. These results demonstrate that the inclusion of at least one DRG de-targeting element (e.g., as described herein) and at least one liver de-targeting element (e.g., as described herein) in the de-targeting region of an RNA transcript can reduce its expression in both DRG and liver without negatively reducing expression in a target tissue (e.g., brain).

### Example 8: DRG De-Targeting in Combinatorial Assay in Non-Human Primates (NHP)

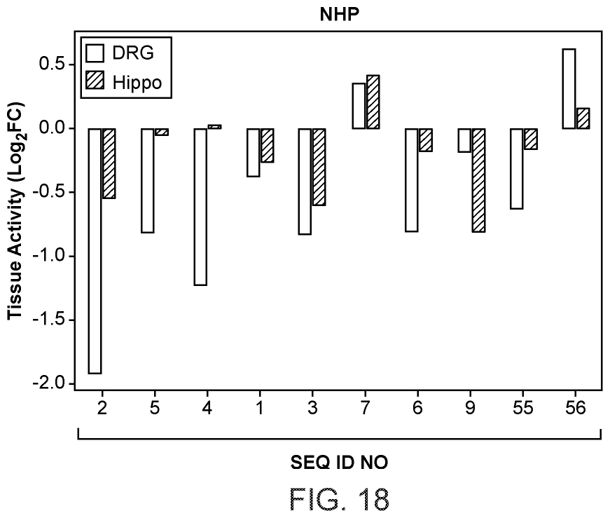

Conservation of the de-targeting activity of the selected DRG de-targeting elements between mouse and non-human primate (NHP) was assessed. A library of AAVs with a modified selection of DRG and liver de-targeting elements was prepared in a manner similar to that described above in Example 7. The library was administered by unilateral intracerebroventricular (ICV) injection at a dose of 10\hat{ }14 viral genomes/animal into juvenile cynomolgus macaques between 16-21 months of age (n=2). Animals were necropsied at approximately 50 days after treatment, and DRG and brain tissue was harvested for DNA and RNA extraction. AAV-driven transcript expression (EGFP-KASH) was evaluated by reverse transcription droplet digital PCR analysis using vector-specific primer/probes. Total RNA expression was normalized to AAV genome copy number in each sample. Regression-based models of differential expression were used to describe de-targeting contribution of each element across all points/instances in recovered library. FIG. 18 shows the results (log₂ of fold-change) for several different DRG de-targeting elements in NHPs. As seen in this FIG. 18, SEQ ID NOs 2, 5, and 4, when present in the de-targeting region of an RNA transcript, have robust DRG de-targeting activity in NHP, a result similar to mice (see FIG. 16). In addition, SEQ ID NO: 6 demonstrates significant DRG de-targeting activity in NHP.

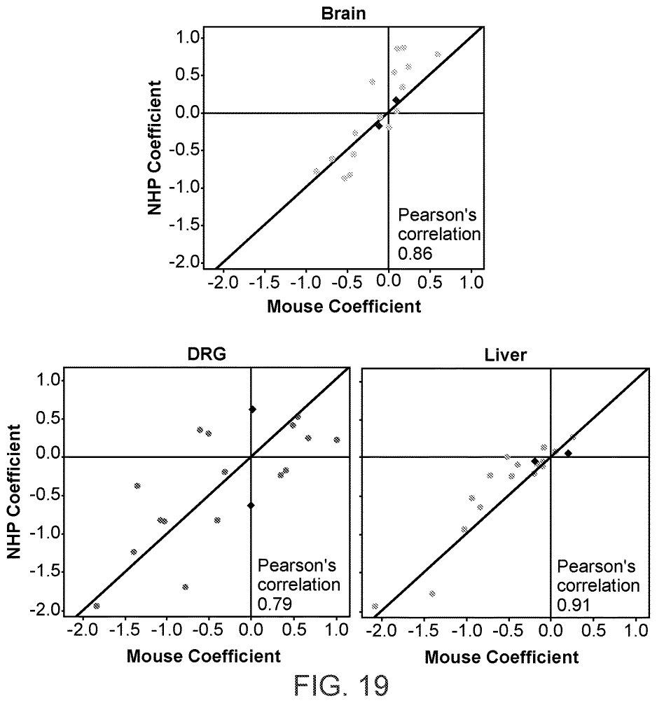

Additional analyses of the DRG and liver de-targeting activities of the elements included in both the mouse and NHP screens was conducted. FIG. 19 shows scatter plots comparing log 2FC expression coefficient for each de-targeting element in mouse and NHP brain tissue (top panel), DRG tissue (bottom left panel) and liver tissue (bottom right panel). The Pearson's correlation between the mouse and NHP data for each of the de-targeting elements was 0.86 for brain expression, 0.91 for liver expression, and 0.79 for DRG expression. These plots show that the effect of the de-targeting elements on transgene expression is correlated between the relevant tissues in mouse and NHP.

Table 8 below provides the combined log 2 coefficients for the mouse and NHP combinatorial de-targeting experiments, which includes mouse and NHP element coefficients in all 3 tissues.

| SEQ ID NO | | | | | | |
|---|---|---|---|---|---|---|
| (RNA in | Mouse | | | NHP | | |
| transcript) | Liver | DRG | Brain | Liver | DRG | Brain |
| 1 | −0.39374 | −1.35307 | −0.39725 | −0.10058 | −0.37649 | −0.26559 |
| 2 | −0.10453 | −1.82981 | −0.41033 | −0.11673 | −1.93134 | −0.54937 |
| 3 | −0.09822 | −1.03319 | −0.67106 | −0.06864 | −0.83251 | −0.60612 |
| 4 | −0.09780 | −1.40056 | 0.11859 | 0.13347 | −1.23464 | 0.02764 |
| 5 | −0.52394 | −1.07545 | −0.10272 | 0.00006 | −0.82307 | −0.05151 |
| 6 | −0.15178 | −0.40836 | 0.01630 | −0.09760 | −0.81596 | −0.17859 |
| 7 | −0.47088 | −0.60594 | −0.18154 | −0.24214 | 0.36110 | 0.42018 |
| 9 | −0.21079 | −0.31458 | −0.45192 | −0.21210 | −0.18583 | −0.81542 |
| 55 | −0.19697 | −0.01108 | −0.10272 | −0.04750 | −0.62688 | −0.16552 |
| 60 | −1.40728 | −0.78155 | −0.52578 | −1.76935 | −1.68699 | −0.86692 |
| 62 | −2.08054 | 0.66717 | 0.11742 | −1.93030 | 0.25003 | 0.86676 |
| 64 | −1.03595 | −0.51267 | −0.86765 | −0.93343 | 0.31841 | −0.77653 |
| 68 | −0.83963 | 0.34322 | 0.25374 | −0.64996 | −0.23860 | 0.62821 |
| 70 | −0.72485 | 0.53554 | 0.18972 | −0.23411 | 0.52444 | 0.87164 |
| 71 | −0.93236 | 0.39871 | 0.07137 | −0.53062 | −0.16893 | 0.54227 |
| 113 | −1.54304 | −0.01440 | −0.00899 | −1.88118 | −1.41060 | 0.01497 |
| 118 | −0.11363 | −2.24094 | −0.28912 | −0.66230 | −2.28249 | −0.14123 |
| 119 | −0.06670 | −2.28995 | 0.03588 | −0.30356 | −2.58043 | 0.18537 |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1-90. (canceled)

91. A nucleic acid cassette comprising a therapeutic transgene encoding an RNA transcript, wherein the RNA transcript comprises a sequence of (i) any of SEQ ID NO. 2 and SEQ ID NOs. 1, 3-10 and 43-48, (ii) a variant, functional fragment, or combination thereof, or (iii) a sequence at least 80% identical to (i) or (ii).

92. The nucleic acid cassette of claim 91, wherein the RNA transcript further comprises a second, third, fourth or five or more sequence of (i), (ii), or (iii).

93. The nucleic acid cassette of claim 91, wherein the sequence of: (i), (ii), or (iii) provides a binding site for one or more of hsa-mir-196b-5p, hsa-mir-10b-5p, hsa-mir-24-2-5p, hsa-mir-183-3p, hsa-mir-196a-5p and hsa-mir-494-3p, optionally wherein the RNA transcript is an mRNA, wherein the sequence of (i), (ii), or (iii) is located in one or more of: a 3′ UTR region of the mRNA, a 5′ UTR of the mRNA, or an intron of the mRNA.

94. The nucleic acid cassette of claim 91, wherein the nucleic acid cassette comprises a CNS-selective promoter, optionally wherein the CNS selective promoter is selected from the group consisting of: Ca2+/calmodulin-dependent kinase subunit α (CaMKII) promoters, synapsin I promoters, 67 kDa glutamic acid decarboxylase (GAD67) promoters, homeobox DIx5/6 promoters, glutamate receptor 1 (GluR1) promoters, preprotachykinin 1 (Tac1) promoters, Neuron-specific enolase (NSE) promoters, dopaminergic receptor 1 (Drd1a) promoters, MAP1B promoters, Tα1 α-tubulin promoters, decarboxylase promoters, dopamine β-hydroxylase promoters, NCAM promoters, HES-5 promoters, α-internexin promoters, peripherin promoters, and GAP-43 promoters, and PaqR4 promoters.

95. The nucleic acid cassette of claim 91, wherein the RNA transcript is for treating a neural disease or disorder, optionally wherein the neural disease or disorder is Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder; optionally wherein the RNA transcript is an mRNA, wherein the mRNA encodes a therapeutic protein selected from (i): a protein encoded by a gene selected from: ALDH7A1, ARHGEF9, ARX, BRAT1, CACNA1A, CACNA1D, CACNB4, CDKL5, CHD2, CHRNA2, CHRNA4, CHRNB2, CLCN2, CLN, CLN2, DEPDC5, DNM1, FGF13, FMR1, FOLR1, FOXG1, GABRA1, GABRB3, GABRD, GABRG2, GRIN2A, GRIN2B, HCN1, HCN4, KCNQ2, KCNQ3, KCNT1, KV3.1, KV3.2, KV3.3, LGI1, MECP2, MEF2C, Myoclonin1/EFHC1, NPRL2, PCDH19, PLCB1, PNKP, POLG1, PRRT2, PTEN, SCN1A, SCN1B, SCN2A, SCN2B, SCN8A, SHANK3, SLC13A5, SLC25A22, SLC2A1, SLC6A1, SLC6A8, SPTAN1, ST3GAL3, STRADA, STXBP1, SYNGAP1, TBC1D24, UBE3A, and WWOX, (ii) a protein having at least 90% sequence identity to (i), (iii) a functional fragment of (i) or (ii), or (iv) a transcription factor which modulates expression of a gene from (i).

96. The nucleic acid cassette of claim 91, wherein the sequence of (i), (ii), or (iii), results in decreased expression of the RNA transcript and/or a polypeptide encoded by the RNA transcript in DRG cells as compared to expression of the RNA transcript and/or polypeptide in DRG cells from an otherwise equivalent RNA transcript without the sequence of (i), (ii), or (iii); optionally wherein the sequence of (i), (ii) or (iii), does not result in decreased expression of the RNA transcript and/or a polypeptide encoded by the RNA transcript in target cells as compared to expression of the RNA transcript and/or polypeptide in target cells from an otherwise equivalent RNA transcript without the sequence of (i), (ii), or (iii).

97. The nucleic acid cassette of claim 96, wherein the target cells are neural cells, optionally wherein the neural cells are cerebrum cells, brainstem cells, hippocampus cells, or cerebellum cells.

98. The nucleic acid cassette of claim 91, wherein the nucleic acid cassette is a viral vector, optionally wherein the viral vector is an adeno-associated virus (AAV) vector.

99. An RNA with a sequence encoded by a nucleic acid cassette of claim 91.

100. A method of decreasing dorsal root ganglion (DRG) expression of an RNA transcript and/or a therapeutic protein encoded thereby, the method comprising including a sequence of (i) any of SEQ ID NOs. 1-10 and 43-48, (ii) a variant, functional fragment, or combination thereof, or (iii) a sequence at least 80% identical to (i) or (ii) in the RNA transcript.

101. A method for expressing a therapeutic RNA transcript, comprising:
administering the nucleic acid cassette of claim 91 to a subject, optionally wherein the subject has a neural disease or disorder, optionally wherein the neural disease or disorder is Alpers-Huttenlocher syndrome, Angelman syndrome, CDKL5 deficiency disorder, Dravet syndrome, Rett syndrome, Parkinson's disease and Parkinson's LIDS (side effect of Parkinson's medication), Alzheimer's disease, creatine transporter deficiency, FOXG1 syndrome, fragile X syndrome, Phelan-McDermid syndrome, childhood absence epilepsy, childhood epilepsy centrotemporal spikes (benign rolandic epilepsy), early myoclonic encephalopathy (EME), epilepsy eyelid myoclonia (Jeavons syndrome), epilepsy of infancy with migrating focal seizures, epilepsy myoclonic absences, epileptic encephalopathy continuous spike and wave during sleep (CSWS), infantile spasms (West syndrome), juvenile myoclonic epilepsy, Landau-Kleffner syndrome, Lennox-Gastaut syndrome (LGS), myoclonic epilepsy in infancy, Ohtahara syndrome, Panayiotopoulos syndrome, progressive myoclonic epilepsy, reflex Epilepsy, self-limited familial and non-familial neonatal infantile seizures, self-limited late onset occipital epilepsy, Gastaut syndrome, epilepsy generalized tonic clonic seizures alone, genetic epilepsy with febrile seizures plus, juvenile absence epilepsy, myoclonic atonic epilepsy (Doose syndrome), sleep-related hypermotor epilepsy (SHE), febrile seizures, focal epilepsy, West syndrome, early onset epilepsy, benign familial infantile epilepsy, or attention deficit-hyperactivity disorder; and optionally wherein the administering by intraparenchymal, intrathecal, intra-cisterna magna, intracerebroventricular or intracranial administration.

102. A nucleic acid cassette comprising a therapeutic transgene encoding an RNA transcript, wherein the RNA transcript comprises a sequence of (i) any SEQ ID NOS. 65, 110, and 112, (ii) a variant, functional fragment, or combination thereof, or (iii) a sequence at least 80% identical to (i) or (ii), wherein the sequence decreases expression of the mRNA in liver cells.

103. A method of decreasing liver expression of a therapeutic RNA transcript and/or a protein encoded thereby, the method comprising including a sequence of (i) any SEQ ID NOS. 65, 110, and 112, (ii) a variant, functional fragment, or combination thereof, or (iii) a sequence at least 80% identical to (i) or (ii) in the RNA transcript.

104. A nucleic acid cassette comprising a therapeutic transgene encoding an RNA transcript that comprises a first sequence that de-targets expression in dorsal root ganglion (DRG) cells and a second sequence that de-targets expression in liver cells, wherein: (a) the first sequence is: (i) any of SEQ ID NOS. 1-10 and 43-48, (ii) a variant, functional fragment, or combination thereof, or (iii) a sequence at least 80% identical to (i) or (ii); and (b) the second sequence is: (iv) any of SEQ ID NOs. 57-62, 64-71, 110, and 112, (v) a variant, functional fragment, or combination thereof, or (vi) a sequence at least 80% identical to (iv) or (v).

105. A method of decreasing dorsal root ganglion (DRG) and liver expression of a therapeutic RNA transcript and/or a protein encoded thereby, the method comprising adding a first and second sequences to the therapeutic RNA transcript, wherein the first sequence de-targets expression in DRG cells and the second sequence de-targets expression in liver cells, wherein: (a) the first sequence is: (i) any of SEQ ID NOS. 1-10 and 43-48, (ii) a variant, functional fragment, or combination thereof, or (iii) a sequence at least 80% identical to (i) or (ii); and (b) the second sequence is: (iv) any of SEQ ID NOs. 57-62, 64-71, 110, and 112, (v) a variant, functional fragment, or combination thereof, or (vi) a sequence at least 80% identical to (iv) or (v).


SEQUENCE LISTING (123 sequences)

SEQ ID NO: 1 - RNA, 22 nt, synthetic construct
  source 1..22: mol_type = other RNA; note = hsa-mir-196b-5p binding site; Sequence in transcript
cccaacaacaggaaactaccta

SEQ ID NO: 2 - RNA, 23 nt, synthetic construct
  source 1..23: mol_type = other RNA; note = hsa-mir-10b-5p binding site; Sequence in transcript
cacaaattcggttctacagggta

SEQ ID NO: 3 - RNA, 22 nt, synthetic construct
  source 1..22: mol_type = other RNA; note = hsa-mir-24-2-5p binding site; Sequence in transcript
ctgtgtttcagctcagtaggca

SEQ ID NO: 4 - RNA, 22 nt, synthetic construct
  source 1..22: mol_type = other RNA; note = hsa-mir-183-3p binding site; Sequence in transcript
ttatggcccttcggtaattcac

SEQ ID NO: 5 - RNA, 22 nt, synthetic construct
  source 1..22: mol_type = other RNA; note = hsa-mir-196a-5p binding site; Sequence in transcript
cccaacaacatgaaactaccta

SEQ ID NO: 6 - RNA, 22 nt, synthetic construct
  source 1..22: mol_type = other RNA; note = hsa-mir-494-3p binding site; Sequence in transcript
gaggtttcccgtgtatgtttca

SEQ ID NO: 7 - RNA, 225 nt, synthetic construct
  source 1..225: mol_type = other RNA; note = 3' UTR Tile; Sequence in transcript
aaaagcaaatgctgactgctgctcacgccctggctgtggatgccaaaaacttactcgatgtcattgaccaagcaagactgaaaatgcttgggcagacgagaccacactgagcctcccctaggagcacgtcttgctaccctcttttgaagatgttctctagccttccaccagcagcgaggaattaaccctgtgtcctcagtcgccagcacttacagctccaacttt

SEQ ID NO: 8 - RNA, 125 nt, synthetic construct
  source 1..125: mol_type = other RNA; note = internal stretch of SEQ 7; Sequence in transcript
ttactcgatgtcattgaccaagcaagactgaaaatgcttgggcagacgagaccacactgagcctcccctaggagcacgtcttgctaccctcttttgaagatgttctctagccttccaccagcagc

SEQ ID NO: 9 - RNA, 225 nt, synthetic construct
  source 1..225: mol_type = other RNA; note = 3' UTR Tile; Sequence in transcript
tctggatggaataactcttcttttgatgatgatcataaaattaaatttcagacttcactagttctgcgtaacacgacaggttgcctaaggaacctcagctccgcgggggaagaagctcggaagcaaatgcggtcctgcgaggggctggtagactcactgttgtatgtgatccacacgtgtgtgaacacatccgattacgacagcaagacggtggagaactgcgtg

SEQ ID NO: 10 - RNA, 125 nt, synthetic construct
  source 1..125: mol_type = other RNA; note = internal stretch of SEQ 9; Sequence in transcript
gacttcactagttctgcgtaacacgacaggttgcctaaggaacctcagctccgcgggggaagaagctcggaagcaaatgcggtcctgcgaggggctggtagactcactgttgtatgtgatccaca

SEQ ID NO: 11 - DNA, 22 bp, synthetic construct
  source 1..22: mol_type = other DNA; note = hsa-mir-196b-5p binding site; Sequence in vector
cccaacaacaggaaactaccta

SEQ ID NO: 12 - DNA, 23 bp, synthetic construct
  source 1..23: mol_type = other DNA; note = hsa-mir-10b-5p binding site; Sequence in vector
cacaaattcggttctacagggta

SEQ ID NO: 13 - DNA, 22 bp, synthetic construct
  source 1..22: mol_type = other DNA; note = hsa-mir-24-2-5p binding site; Sequence in vector
ctgtgtttcagctcagtaggca

SEQ ID NO: 14 - DNA, 22 bp, synthetic construct
  source 1..22: mol_type = other DNA; note = hsa-mir-183-3p binding site; Sequence in vector
ttatggcccttcggtaattcac

SEQ ID NO: 15 - DNA, 22 bp, synthetic construct
  source 1..22: mol_type = other DNA; note = hsa-mir-196a-5p binding site; Sequence in vector
cccaacaacatgaaactaccta

SEQ ID NO: 16 - DNA, 22 bp, synthetic construct
  source 1..22: mol_type = other DNA; note = hsa-mir-494-3p binding site; Sequence in vector
gaggtttcccgtgtatgtttca

SEQ ID NO: 17 - DNA, 225 bp, synthetic construct
  source 1..225: mol_type = other DNA; note = 3' UTR Tile; Sequence in vector
aaaagcaaatgctgactgctgctcacgccctggctgtggatgccaaaaacttactcgatgtcattgaccaagcaagactgaaaatgcttgggcagacgagaccacactgagcctcccctaggagcacgtcttgctaccctcttttgaagatgttctctagccttccaccagcagcgaggaattaaccctgtgtcctcagtcgccagcacttacagctccaacttt

SEQ ID NO: 18 - DNA, 125 bp, synthetic construct
  source 1..125: mol_type = other DNA; note = internal stretch of SEQ 17; Sequence in vector
ttactcgatgtcattgaccaagcaagactgaaaatgcttgggcagacgagaccacactgagcctcccctaggagcacgtcttgctaccctcttttgaagatgttctctagccttccaccagcagc

SEQ ID NO: 19 - DNA, 225 bp, synthetic construct
  source 1..225: mol_type = other DNA; note = 3' UTR Tile; Sequence in vector
tctggatggaataactcttcttttgatgatgatcataaaattaaatttcagacttcactagttctgcgtaacacgacaggttgcctaaggaacctcagctccgcgggggaagaagctcggaagcaaatgcggtcctgcgaggggctggtagactcactgttgtatgtgatccacacgtgtgtgaacacatccgattacgacagcaagacggtggagaactgcgtg

SEQ ID NO: 20 - DNA, 125 bp, synthetic construct
  source 1..125: mol_type = other DNA; note = internal stretch of SEQ 19; Sequence in vector
gacttcactagttctgcgtaacacgacaggttgcctaaggaacctcagctccgcgggggaagaagctcggaagcaaatgcggtcctgcgaggggctggtagactcactgttgtatgtgatccaca

SEQ ID NO: 21 - RNA, 22 nt, Homo sapiens
  source 1..22: mol_type = transcribed RNA; note = hsa-mir-196b-5p
taggtagtttcctgttgttggg

SEQ ID NO: 22 - RNA, 23 nt, Homo sapiens
  source 1..23: mol_type = transcribed RNA; note = hsa-mir-10b-5p
taccctgtagaaccgaatttgtg

SEQ ID NO: 23 - RNA, 22 nt, Homo sapiens
  source 1..22: mol_type = transcribed RNA; note = hsa-mir-24-2-5p
tgcctactgagctgaaacacag

SEQ ID NO: 24 - RNA, 22 nt, Homo sapiens
  source 1..22: mol_type = transcribed RNA; note = hsa-mir-183-3p
gtgaattaccgaagggccataa

SEQ ID NO: 25 - RNA, 22 nt, Homo sapiens
  source 1..22: mol_type = transcribed RNA; note = hsa-mir-196a-5p
taggtagtttcatgttgttggg

SEQ ID NO: 26 - RNA, 22 nt, Homo sapiens
  source 1..22: mol_type = transcribed RNA; note = hsa-mir-494-3p
tgaaacatacacgggaaacctc

SEQ ID NO: 27 - RNA, 84 nt, Homo sapiens
  source 1..84: mol_type = transcribed RNA; note = hsa-mir-196b pre-miRNA
actggtcggtgatttaggtagtttcctgttgttgggatccacctttctctcgacagcacgacactgccttcattacttcagttg

SEQ ID NO: 28 - RNA, 110 nt, Homo sapiens
  source 1..110: mol_type = transcribed RNA; note = hsa-mir-10b pre-miRNA
ccagaggttgtaacgttgtctatatataccctgtagaaccgaatttgtgtggtatccgtatagtcacagattcgattctaggggaatatatggtcgatgcaaaaacttca

SEQ ID NO: 29 - RNA, 73 nt, Homo sapiens
  source 1..73: mol_type = transcribed RNA; note = hsa-mir-24-2 pre-miRNA
ctctgcctcccgtgcctactgagctgaaacacagttggtttgtgtacactggctcagttcagcaggaacaggg

SEQ ID NO: 30 - RNA, 110 nt, Homo sapiens
  source 1..110: mol_type = transcribed RNA; note = hsa-mir-183 pre-miRNA
ccgcagagtgtgactcctgttctgtgtatggcactggtagaattcactgtgaacagtctcagtcagtgaattaccgaagggccataaacagagcagagacagatccacga

SEQ ID NO: 31 - RNA, 70 nt, Homo sapiens
  source 1..70: mol_type = transcribed RNA; note = hsa-mir-196a-1 pre-miRNA
gtgaattaggtagtttcatgttgttgggcctgggtttctgaacacaacaacattaaaccacccgattcac

SEQ ID NO: 32 - RNA, 110 nt, Homo sapiens
  source 1..110: mol_type = transcribed RNA; note = hsa-mir-196a-2 pre-miRNA
tgctcgctcagctgatctgtggcttaggtagtttcatgttgttgggattgagttttgaactcggcaacaagaaactgcctgagttacatcagtcggttttcgtcgagggc

SEQ ID NO: 33 - RNA, 81 nt, Homo sapiens
  source 1..81: mol_type = transcribed RNA; note = hsa-mir-494 pre-miRNA
gatactcgaaggagaggttgtccgtgttgtcttctctttatttatgatgaaacatacacgggaaacctcttttttagtatc

SEQ ID NO: 34 - DNA, 130 bp, synthetic construct
  source 1..130: mol_type = other DNA; note = 5' AAV ITR; Sequence in vector
cctgcaggcagctgcgcgctcgctcgctcactgaggccgcccgggcgtcgggcgacctttggtcgcccggcctcagtgagcgagcgagcgcgcagagagggagtggccaactccatcactaggggttcct

SEQ ID NO: 35 - DNA, 141 bp, synthetic construct
  source 1..141: mol_type = other DNA; note = 3' AAV ITR; Sequence in vector
aggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcagg

SEQ ID NO: 36 - RNA, 22 nt, Homo sapiens
  source 1..22: mol_type = transcribed RNA; note = hsa-mir-196b-3p
tcgacagcacgacactgccttc

SEQ ID NO: 37 - RNA, 22 nt, Homo sapiens
  source 1..22: mol_type = transcribed RNA; note = hsa-mir-10b-3p
acagattcgattctaggggaat

SEQ ID NO: 38 - RNA, 22 nt, Homo sapiens
  source 1..22: mol_type = transcribed RNA; note = hsa-mir-24-2-3p
tggctcagttcagcaggaacag

SEQ ID NO: 39 - RNA, 22 nt, Homo sapiens
  source 1..22: mol_type = transcribed RNA; note = hsa-mir-183-5p
tatggcactggtagaattcact

SEQ ID NO: 40 - RNA, 21 nt, Homo sapiens
  source 1..21: mol_type = transcribed RNA; note = hsa-mir-196a-1-3p
caacaacattaaaccacccga

SEQ ID NO: 41 - RNA, 22 nt, Homo sapiens
  source 1..22: mol_type = transcribed RNA; note = hsa-mir-196a-2-3p
cggcaacaagaaactgcctgag

SEQ ID NO: 42 - RNA, 23 nt, Homo sapiens
  source 1..23: mol_type = transcribed RNA; note = hsa-mir-494-5p
aggttgtccgtgttgtcttctct

SEQ ID NO: 43 - RNA, 154 nt, synthetic construct
  source 1..154: mol_type = other RNA; note = 4xSEQ ID NO:1; Sequence in transcript
cccaacaacaggaaactacctatggcgagacctccacccctcctcccaacaacaggaaactacctagttgataagtcggggacaatctcccaacaacaggaaactacctagccgtcatgaacgcaccagtcccccaacaacaggaaactaccta

SEQ ID NO: 44 - RNA, 154 nt, synthetic construct
  source 1..154: mol_type = other RNA; note = 4xSEQ ID NO:4; Sequence in transcript
ttatggcccttcggtaattcactggcgagacctccacccctcctttatggcccttcggtaattcacgttgataagtcggggacaatctttatggcccttcggtaattcacgccgtcatgaacgcaccagtccttatggcccttcggtaattcac

SEQ ID NO: 45 - RNA, 154 nt, synthetic construct
  source 1..154: mol_type = other RNA; note = 4xSEQ ID NO:3; Sequence in transcript
ctgtgtttcagctcagtaggcatggcgagacctccacccctcctctgtgtttcagctcagtaggcagttgataagtcggggacaatctctgtgtttcagctcagtaggcagccgtcatgaacgcaccagtccctgtgtttcagctcagtaggca

SEQ ID NO: 46 - RNA, 155 nt, synthetic construct
  source 1..155: mol_type = other RNA; note = 4xSEQ ID NO:2; Sequence in transcript
cacaaattcggttctacagggtaggcgagacctccacccctcctcacaaattcggttctacagggtattgataagtcggggacaatctcacaaattcggttctacagggtaccgtcatgaacgcaccagtcccacaaattcggttctacagggta

SEQ ID NO: 47 - RNA, 156 nt, synthetic construct
  source 1..156: mol_type = other RNA; note = 2x(SEQ ID NO:3 + SEQ ID NO:2); Sequence in transcript
ctgtgtttcagctcagtaggcatggcgagacctccacccctcctcacaaattcggttctacagggtagttgataagtcggggacaatctctgtgtttcagctcagtaggcagccgtcatgaacgcaccagtcccacaaattcggttctacagggta

SEQ ID NO: 48 - RNA, 156 nt, synthetic construct
  source 1..156: mol_type = other RNA; note = 2x(SEQ ID NO:1 + SEQ ID NO:2); Sequence in transcript
cccaacaacaggaaactacctatggcgagacctccacccctcctcacaaattcggttctacagggtattgataagtcggggacaatctacccaacaacaggaaactacctaccgtcatgaacgcaccagtcctcacaaattcggttctacagggta

SEQ ID NO: 49 - DNA, 154 bp, synthetic construct
  source 1..154: mol_type = other DNA; note = 4xSEQ ID NO:1; Sequence in vector
cccaacaacaggaaactacctatggcgagacctccacccctcctcccaacaacaggaaactacctagttgataagtcggggacaatctcccaacaacaggaaactacctagccgtcatgaacgcaccagtcccccaacaacaggaaactaccta

SEQ ID NO: 50 - DNA, 154 bp, synthetic construct
  source 1..154: mol_type = other DNA; note = 4xSEQ ID NO:4; Sequence in vector
ttatggcccttcggtaattcactggcgagacctccacccctcctttatggcccttcggtaattcacgttgataagtcggggacaatctttatggcccttcggtaattcacgccgtcatgaacgcaccagtccttatggcccttcggtaattcac

SEQ ID NO: 51 - DNA, 154 bp, synthetic construct
  source 1..154: mol_type = other DNA; note = 4xSEQ ID NO:3; Sequence in vector
ctgtgtttcagctcagtaggcatggcgagacctccacccctcctctgtgtttcagctcagtaggcagttgataagtcggggacaatctctgtgtttcagctcagtaggcagccgtcatgaacgcaccagtccctgtgtttcagctcagtaggca

SEQ ID NO: 52 - DNA, 155 bp, synthetic construct
  source 1..155: mol_type = other DNA; note = 4xSEQ ID NO:2; Sequence in vector
cacaaattcggttctacagggtaggcgagacctccacccctcctcacaaattcggttctacagggtattgataagtcggggacaatctcacaaattcggttctacagggtaccgtcatgaacgcaccagtcccacaaattcggttctacagggta

SEQ ID NO: 53 - DNA, 156 bp, synthetic construct
  source 1..156: mol_type = other DNA; note = 2x(SEQ ID NO:3 + SEQ ID NO:2); Sequence in vector
ctgtgtttcagctcagtaggcatggcgagacctccacccctcctcacaaattcggttctacagggtagttgataagtcggggacaatctctgtgtttcagctcagtaggcagccgtcatgaacgcaccagtcccacaaattcggttctacagggta

SEQ ID NO: 54 - DNA, 156 bp, synthetic construct
  source 1..156: mol_type = other DNA; note = 2x(SEQ ID NO:1 + SEQ ID NO:2); Sequence in vector
cccaacaacaggaaactacctatggcgagacctccacccctcctcacaaattcggttctacagggtattgataagtcggggacaatctacccaacaacaggaaactacctaccgtcatgaacgcaccagtcctcacaaattcggttctacagggta

SEQ ID NO: 55 - DNA, 127 bp, synthetic construct
  source 1..127: mol_type = other DNA; note = Random 1 Sequence in vector
gcacctgtctaaaaggcactattccccatacaatggtccaaagtctggcagttaccatccgaccgcgttttctgcggagcccaggatactttcttacttaagcttgtgacggcggtagagcatgaaa

SEQ ID NO: 56 - DNA, 127 bp, synthetic construct
  source 1..127: mol_type = other DNA; note = Random 2 Sequence in vector
tgcgtgagtgttgggaataatagctaagaggggcacaagcgagatcagaagcgcaagtgctacggagcctatctttattgtactgagtctactataggcgacgggtacgttacaagactatcatagc

SEQ ID NO: 57 - RNA, 22 nt, synthetic construct
  source 1..22: mol_type = other RNA; note = hsa-mir-22-3p binding site; Sequence in transcript
acagttcttcaactggcagctt

SEQ ID NO: 58 - RNA, 19 nt, synthetic construct
  source 1..19: mol_type = other RNA; note = hsa-mir-1258 binding site; Sequence in transcript
ccacgacctaatcctaact

SEQ ID NO: 59 - RNA, 21 nt, synthetic construct
  source 1..21: mol_type = other RNA; note = hsa-mir-5589-3p binding site; Sequence in transcript
gggagctaggttgccatgtgc

SEQ ID NO: 60 - RNA, 23 nt, synthetic construct
  source 1..23: mol_type = other RNA; note = hsa-mir-17-5p binding site; Sequence in transcript
ctacctgcactgtaagcactttg

SEQ ID NO: 61 - RNA, 22 nt, synthetic construct
  source 1..22: mol_type = other RNA; note = hsa-mir-203a-3p binding site; Sequence in transcript
ctagtggtcctaaacatttcac

SEQ ID NO: 62 - RNA, 20 nt, synthetic construct
  source 1..20: mol_type = other RNA; note = hsa-mir-122-3p binding site; Sequence in transcript
atttagtgtgataatggcgt

SEQ ID NO: 63 - RNA, 22 nt, synthetic construct
  source 1..22: mol_type = other RNA; note = hsa-mir-122-5p binding site; Sequence in transcript
caaacaccattgtcacactcca

SEQ ID NO: 64 - RNA, 23 nt, synthetic construct
  source 1..23: mol_type = other RNA; note = hsa-mir-93-5p binding site; Sequence in transcript
ctacctgcacgaacagcactttg

SEQ ID NO: 65 - RNA, 23 nt, synthetic construct
  source 1..23: mol_type = other RNA; note = hsa-mir-19a-3p binding site; Sequence in transcript
tcagttttgcatagatttgcaca

SEQ ID NO: 66 - RNA, 177 nt, synthetic construct
  source 1..177: mol_type = other RNA; note = 3prime UTR Tile; Sequence in transcript
gagatcatatgcatatgtgtagggctggagcgtgtgtgtgtcttgagattgtgtgtgttgcagtcatcatatctatgtgttacagattgtgtatgttagccttgtgtatgtgtgcttgattgaggtggtgtatttgggttgaaattgtgtcatatgtgtgtgctatccatctcgtgt

SEQ ID NO: 67 - RNA, 180 nt, synthetic construct
  source 1..180: mol_type = other RNA; note = 3prime UTR Tile; Sequence in transcript
gcagaagaggtaagggagtgaggcactcctatcccagtctcccaggtttggttgagggctccccaaggcagggcaagatagcggccctgtcactgaccctggcctgtggtggtctgagctggggagggaaggacaccaatgaatcagcttgggacctctttaggccttccccttttcctc

SEQ ID NO: 68 - RNA, 228 nt, synthetic construct
  source 1..228: mol_type = other RNA; note = 3prime UTR Tile; Sequence in transcript
gcattaaaacacacatacaaacaaaatcaaaaacactgcggactttcactcaagctggtctttcttccccagtgtaaggcaatcctgcctactaacaacaccaacaacaaaacactccatctgtgaaagctgacgcagttaagggggctaggcagggcatttgtgccaactaagaatcaccagatacccaccataagtacctatcgcagttttgaagtcgtttctccc

SEQ ID NO: 69 - RNA, 177 nt, synthetic construct
  source 1..177: mol_type = other RNA; note = 3prime UTR Tile; Sequence in transcript
caaccatcacgtcaggctgcccatccaatagactcctgggatggggcagccaaccctggctcatctcatctgccgcttggtgcgtgtgcgtgtgcgtgcatgtgcgtgtgcgtgtgtgcaggggtgagaatctggcagatggtgcctctgcctgctcttcttcgcctcctttattta

SEQ ID NO: 70 - RNA, 152 nt, synthetic construct
  source 1..152: mol_type = other RNA; note = 3prime UTR Tile; Sequence in transcript
tggccctgcgcatgctgaaataactggaacccagcctctcctcctacaccggcctacccatctgggcccaagagctgcactcacactcctacaacgaaggacaaactgtccaggtcggagggatcacgagacacagaacctggaggggtgtg

SEQ ID NO: 71 - RNA, 227 nt, synthetic construct
  source 1..227: mol_type = other RNA; note = 3prime UTR Tile; Sequence in transcript
tctgacttgagagctcccccagtcagatctcaggcttgtccccctgtcagctgcctccagaagggaaggtagccagtgcctgagaagacagtcccttttctacccaccgcactccataacctccatcttctcccacactgatggcgagcagcccctgagcactttctgggactgggagactgcttggtgttccctgaggacaagagacatcctgacagtgttgggca

SEQ ID NO: 72 - DNA, 22 bp, synthetic construct
  source 1..22: mol_type = other DNA; note = hsa-mir-22-3p binding site; Sequence in vector
acagttcttcaactggcagctt

SEQ ID NO: 73 - DNA, 19 bp, synthetic construct
  source 1..19: mol_type = other DNA; note = hsa-mir-1258 binding site; Sequence in vector
ccacgacctaatcctaact

SEQ ID NO: 74 - DNA, 21 bp, synthetic construct
  source 1..21: mol_type = other DNA; note = hsa-mir-5589-3p binding site; Sequence in vector (used in screen)
gggagctaggttgccatgtgc

SEQ ID NO: 75 - DNA, 23 bp, synthetic construct
  source 1..23: mol_type = other DNA; note = hsa-mir-17-5p binding site; Sequence in vector
ctacctgcactgtaagcactttg

SEQ ID NO: 76 - DNA, 22 bp, synthetic construct
  source 1..22: mol_type = other DNA; note = hsa-mir-203a-3p binding site; Sequence in vector
ctagtggtcctaaacatttcac

SEQ ID NO: 77 - DNA, 20 bp, synthetic construct
  source 1..20: mol_type = other DNA; note = hsa-mir-122-3p binding site; Sequence in vector
atttagtgtgataatggcgt

SEQ ID NO: 78 - DNA, 22 bp, synthetic construct
  source 1..22: mol_type = other DNA; note = hsa-mir-122-5p binding site; Sequence in vector
caaacaccattgtcacactcca

SEQ ID NO: 79 - DNA, 23 bp, synthetic construct
  source 1..23: mol_type = other DNA; note = hsa-mir-93-5p binding site; Sequence in vector
ctacctgcacgaacagcactttg

SEQ ID NO: 80 - DNA, 23 bp, synthetic construct
  source 1..23: mol_type = other DNA; note = hsa-mir-19a-3p binding site; Sequence in vector
tcagttttgcatagatttgcaca

SEQ ID NO: 81 - DNA, 177 bp, synthetic construct
  source 1..177: mol_type = other DNA; note = 3prime UTR Tile; Sequence in vector
gagatcatatgcatatgtgtagggctggagcgtgtgtgtgtcttgagattgtgtgtgttgcagtcatcatatctatgtgttacagattgtgtatgttagccttgtgtatgtgtgcttgattgaggtggtgtatttgggttgaaattgtgtcatatgtgtgtgctatccatctcgtgt

SEQ ID NO: 82 - DNA, 180 bp, synthetic construct
  source 1..180: mol_type = other DNA; note = 3prime UTR Tile; Sequence in vector
gcagaagaggtaagggagtgaggcactcctatcccagtctcccaggtttggttgagggctccccaaggcagggcaagatagcggccctgtcactgaccctggcctgtggtggtctgagctggggagggaaggacaccaatgaatcagcttgggacctctttaggccttccccttttcctc

SEQ ID NO: 83 - DNA, 228 bp, synthetic construct
  source 1..228: mol_type = other DNA; note = 3prime UTR Tile; Sequence in vector
gcattaaaacacacatacaaacaaaatcaaaaacactgcggactttcactcaagctggtctttcttccccagtgtaaggcaatcctgcctactaacaacaccaacaacaaaacactccatctgtgaaagctgacgcagttaagggggctaggcagggcatttgtgccaactaagaatcaccagatacccaccataagtacctatcgcagttttgaagtcgtttctccc

SEQ ID NO: 84 - DNA, 177 bp, synthetic construct
  source 1..177: mol_type = other DNA; note = 3prime UTR Tile; Sequence in vector
caaccatcacgtcaggctgcccatccaatagactcctgggatggggcagccaaccctggctcatctcatctgccgcttggtgcgtgtgcgtgtgcgtgcatgtgcgtgtgcgtgtgtgcaggggtgagaatctggcagatggtgcctctgcctgctcttcttcgcctcctttattta

SEQ ID NO: 85 - DNA, 152 bp, synthetic construct
  source 1..152: mol_type = other DNA; note = 3prime UTR Tile; Sequence in vector
tggccctgcgcatgctgaaataactggaacccagcctctcctcctacaccggcctacccatctgggcccaagagctgcactcacactcctacaacgaaggacaaactgtccaggtcggagggatcacgagacacagaacctggaggggtgtg

SEQ ID NO: 86 - DNA, 227 bp, synthetic construct
  source 1..227: mol_type = other DNA; note = 3prime UTR Tile; Sequence in vector
tctgacttgagagctcccccagtcagatctcaggcttgtccccctgtcagctgcctccagaagggaaggtagccagtgcctgagaagacagtcccttttctacccaccgcactccataacctccatcttctcccacactgatggcgagcagcccctgagcactttctgggactgggagactgcttggtgttccctgaggacaagagacatcctgacagtgttgggca

SEQ ID NO: 87 - RNA, 22 nt, Homo sapiens
  source 1..22: mol_type = transcribed RNA; note = hsa-mir-22-3p
aagctgccagttgaagaactgt

SEQ ID NO: 88 - RNA, 21 nt, Homo sapiens
  source 1..21: mol_type = transcribed RNA; note = hsa-mir-1258
agttaggattaggtcgtggaa

SEQ ID NO: 89 - RNA, 21 nt, Homo sapiens
  source 1..21: mol_type = transcribed RNA; note = hsa-mir-5589-3p
gcacatggcaacctagctccc

SEQ ID NO: 90 - RNA, 23 nt, Homo sapiens
  source 1..23: mol_type = transcribed RNA; note = hsa-mir-17-5p
caaagtgcttacagtgcaggtag

SEQ ID NO: 91 - RNA, 22 nt, Homo sapiens
  source 1..22: mol_type = transcribed RNA; note = hsa-mir-203a-3p
gtgaaatgtttaggaccactag

SEQ ID NO: 92 - RNA, 21 nt, Homo sapiens
  source 1..21: mol_type = transcribed RNA; note = hsa-mir-122-3p
aacgccattatcacactaaat

SEQ ID NO: 93 - RNA, 22 nt, Homo sapiens
  source 1..22: mol_type = transcribed RNA; note = hsa-mir-122-5p
tggagtgtgacaatggtgtttg

SEQ ID NO: 94 - RNA, 23 nt, Homo sapiens
  source 1..23: mol_type = transcribed RNA; note = hsa-mir-93-5p
caaagtgctgttcgtgcaggtag

SEQ ID NO: 95 - RNA, 23 nt, Homo sapiens
  source 1..23: mol_type = transcribed RNA; note = hsa-mir-19a-3p
tgtgcaaatctatgcaaaactga

SEQ ID NO: 96 - RNA, 85 nt, Homo sapiens
  source 1..85: mol_type = transcribed RNA; note = hsa-mir-22 pre-miRNA
ggctgagccgcagtagttcttcagtggcaagctttatgtcctgacccagctaaagctgccagttgaagaactgttgccctctgcc

SEQ ID NO: 97 - RNA, 73 nt, Homo sapiens
  source 1..73: mol_type = transcribed RNA; note = hsa-mir-1258 pre-miRNA
ctgtggcttccacgacctaatcctaactcctgcgagtccctggagttaggattaggtcgtggaagccacagga

SEQ ID NO: 98 - RNA, 60 nt, Homo sapiens
  source 1..60: mol_type = transcribed RNA; note = hsa-mir-5589 pre-miRNA
ggctgggtgctcttgtgcagtgagcaacctacacaactgcacatggcaacctagctccca

SEQ ID NO: 99 - RNA, 84 nt, Homo sapiens
  source 1..84: mol_type = transcribed RNA; note = hsa-mir-17 pre-miRNA
gtcagaataatgtcaaagtgcttacagtgcaggtagtgatatgtgcatctactgcagtgaaggcacttgtagcattatggtgac

SEQ ID NO: 100 - RNA, 110 nt, Homo sapiens
  source 1..110: mol_type = transcribed RNA; note = hsa-mir-203a pre-miRNA
gtgttggggactcgcgcgctgggtccagtggttcttaacagttcaacagttctgtagcgcaattgtgaaatgtttaggaccactagacccggcgggcgcggcgacagcga

SEQ ID NO: 101 - RNA, 85 nt, Homo sapiens
  source 1..85: mol_type = transcribed RNA; note = hsa-mir-122 pre-miRNA
ccttagcagagctgtggagtgtgacaatggtgtttgtgtctaaactatcaaacgccattatcacactaaatagctactgctaggc

SEQ ID NO: 102 - RNA, 80 nt, Homo sapiens
  source 1..80: mol_type = transcribed RNA; note = hsa-mir-93 pre-miRNA
ctgggggctccaaagtgctgttcgtgcaggtagtgtgattacccaacctactgctgagctagcacttcccgagcccccgg

SEQ ID NO: 103 - RNA, 82 nt, Homo sapiens
  source 1..82: mol_type = transcribed RNA; note = hsa-mir-19a pre-miRNA
gcagtcctctgttagttttgcatagttgcactacaagaagaatgtagttgtgcaaatctatgcaaaactgatggtggcctgc

SEQ ID NO: 104 - RNA, 22 nt, Homo sapiens
  source 1..22: mol_type = transcribed RNA; note = hsa-mir-22-5p
agttcttcagtggcaagcttta

SEQ ID NO: 105 - RNA, 21 nt, Homo sapiens
  source 1..21: mol_type = transcribed RNA; note = hsa-mir-5589-5p
ggctgggtgctcttgtgcagt

SEQ ID NO: 106 - RNA, 22 nt, Homo sapiens
  source 1..22: mol_type = transcribed RNA; note = hsa-mir-17-3p
actgcagtgaaggcacttgtag

SEQ ID NO: 107 - RNA, 25 nt, Homo sapiens
  source 1..25: mol_type = transcribed RNA; note = hsa-mir-203a-5p
agtggttcttaacagttcaacagtt

SEQ ID NO: 108 - RNA, 22 nt, Homo sapiens
  source 1..22: mol_type = transcribed RNA; note = hsa-mir-93-3p
actgctgagctagcacttcccg

SEQ ID NO: 109 - RNA, 22 nt, Homo sapiens
  source 1..22: mol_type = transcribed RNA; note = hsa-mir-19a-5p
agttttgcatagttgcactaca

SEQ ID NO: 110 - RNA, 241 nt, synthetic construct
  source 1..241: mol_type = other RNA; note = 2x (SEQ ID NO:65 + SEQ ID NO:58 + SEQ ID NO:60) binding site; Sequence in transcript
tcagttttgcatagatttgcacaaatggtccgcacatggcaacctagctcccgaccgcgccacgacctaatcctaactactttcttctacctgcactgtaagcactttgtagagcatgaaagcacctgtctatcagttttgcatagatttgcacaaatggtccgcacatggcaacctagctcccgaccgcgccacgacctaatcctaactactttcttctacctgcactgtaagcactttg

SEQ ID NO: 111 - RNA, 470 nt, synthetic construct
  source 1..470: mol_type = other RNA; note = 2x (SEQ ID NO:1 + SEQ ID NO:62) + SEQ ID NO:8 + 2x (SEQ ID NO:1 + SEQ ID NO:62) + SEQ ID NO:70; Sequence in transcript
cccaacaacaggaaactacctaattatttagtgtgataatggcgttatcccaacaacaggaaactacctagtatttagtgtgataatggcgttttactcgatgtcattgaccaagcaagactgaaaatgcttgggcagacgagaccacactgagcctcccctaggagcacgtcttgctaccctcttttgaagatgttctctagccttccaccagcagccccaacaacaggaaactacctaggtatttagtgtgataatggcgttgtcccaacaacaggaaactacctagatatttagtgtgataatggcgtttggccctgcgcatgctgaaataactggaacccagcctctcctcctacaccggcctacccatctgggcccaagagctgcactcacactcctacaacgaaggacaaactgtccaggtcggagggatcacgagacacagaacctggaggggtgtgactagt

SEQ ID NO: 112 - RNA, 82 nt, synthetic construct
  source 1..82: mol_type = other RNA; note = 3x SEQ ID NO:62; Sequence in transcript
tatttagtgtgataatggcgttaatggtcctatttagtgtgataatggcgttcgaccgcgtatttagtgtgataatggcgtt

SEQ ID NO: 113 - RNA, 54 nt, synthetic construct
  source 1..54: mol_type = other RNA; note = 3x internal part of SEQ ID NO:63; Sequence in transcript
acaccattgtcacactccacaccattgtcacactccacaccattgtcacactcc

SEQ ID NO: 114 - DNA, 241 bp, synthetic construct
  source 1..241: mol_type = other DNA; note = 2x (SEQ ID NO:65 + SEQ ID NO:58 + SEQ ID NO:60); Sequence in vector
tcagttttgcatagatttgcacaaatggtccgcacatggcaacctagctcccgaccgcgccacgacctaatcctaactactttcttctacctgcactgtaagcactttgtagagcatgaaagcacctgtctatcagttttgcatagatttgcacaaatggtccgcacatggcaacctagctcccgaccgcgccacgacctaatcctaactactttcttctacctgcactgtaagcactttg

SEQ ID NO: 115 - DNA, 470 bp, synthetic construct
  source 1..470: mol_type = other DNA; note = 2x (SEQ ID NO:1 + SEQ ID NO:62) + SEQ ID NO:8 + 2x (SEQ ID NO:1 + SEQ ID NO:62) + SEQ ID NO:70; Sequence in vector
cccaacaacaggaaactacctaattatttagtgtgataatggcgttatcccaacaacaggaaactacctagtatttagtgtgataatggcgttttactcgatgtcattgaccaagcaagactgaaaatgcttgggcagacgagaccacactgagcctcccctaggagcacgtcttgctaccctcttttgaagatgttctctagccttccaccagcagccccaacaacaggaaactacctaggtatttagtgtgataatggcgttgtcccaacaacaggaaactacctagatatttagtgtgataatggcgtttggccctgcgcatgctgaaataactggaacccagcctctcctcctacaccggcctacccatctgggcccaagagctgcactcacactcctacaacgaaggacaaactgtccaggtcggagggatcacgagacacagaacctggaggggtgtgactagt

SEQ ID NO: 116 - DNA, 82 bp, synthetic construct
  source 1..82: mol_type = other DNA; note = 3x SEQ ID NO:62; Sequence in vector
tatttagtgtgataatggcgttaatggtcctatttagtgtgataatggcgttcgaccgcgtatttagtgtgataatggcgtt

SEQ ID NO: 117 - DNA, 54 bp, synthetic construct
  source 1..54: mol_type = other DNA; note = 3x internal part of SEQ ID NO:63; Sequence in vector
acaccattgtcacactccacaccattgtcacactccacaccattgtcacactcc

SEQ ID NO: 118 - RNA, 23 nt, synthetic construct
  source 1..23: mol_type = other RNA; note = hsa-mir-96-5p binding site; Sequence in transcript
agcaaaaatgtgctagtgccaaa

SEQ ID NO: 119 - RNA, 24 nt, synthetic construct
  source 1..24: mol_type = other RNA; note = hsa-mir-182-5p binding site; Sequence in transcript
agtgtgagttctaccattgccaaa

SEQ ID NO: 120 - DNA, 23 bp, synthetic construct
  source 1..23: mol_type = other DNA; note = hsa-mir-96-5p binding site; Sequence in vector
agcaaaaatgtgctagtgccaaa

SEQ ID NO: 121 - DNA, 24 bp, synthetic construct
  source 1..24: mol_type = other DNA; note = hsa-mir-182-5p binding site; Sequence in vector
agtgtgagttctaccattgccaaa

SEQ ID NO: 122 - RNA, 78 nt, Homo sapiens
  source 1..78: mol_type = transcribed RNA; note = hsa-mir-96 pre-miRNA
tggccgattttggcactagcacatttttgcttgtgtctctccgctctgagcaatcatgtgcagtgccaatatgggaaa

SEQ ID NO: 123 - RNA, 110 nt, Homo sapiens
  source 1..110: mol_type = transcribed RNA; note = hsa-mir-182 pre-miRNA
gagctgcttgcctccccccgtttttggcaatggtagaactcacactggtgaggtaacaggatccggtggttctagacttgccaactatggggcgaggactcagccggcac